(19) United States
(12) Patent Application Publication
(10) Pub. No.: US 2025/0066450 A1
(43) Pub. Date: Feb. 27, 2025

(54) SOLUBLE BONE MORPHOGENETIC PROTEIN (BMP) RECEPTOR TYPE-1B PROTEINS AND USES THEREOF

(71) Applicant: Lask Pharma, Inc., Waltham, MA (US)

(72) Inventor: John Knopf, Waltham, MA (US)

(21) Appl. No.: 18/793,747

(22) Filed: Aug. 2, 2024

Related U.S. Application Data
(60) Provisional application No. 63/530,928, filed on Aug. 4, 2023.

(51) Int. Cl. C07K 14/71; A61K 38/00; A61P 25/02

(52) U.S. Cl. CPC C07K 14/71; A61P 25/02; A61K 38/00; C07K 2319/30

(57) ABSTRACT

This application relates generally to the field of Bone Morphogenetic Protein (BMP) antagonists (soluble ALK6 fusion proteins), compositions thereof, and methods for use in treating neurodegenerative diseases and/or demyelinating diseases.

### RELATED APPLICATIONS

This application claims priority to U.S. Provisional Application. No. 63/530,928 filed on 4 Aug. 2023, which is incorporated by reference into this application in its entirely.

### SEQUENCE LISTING

The instant application contains a sequence listing which has been submitted electronically in XML format via the USPTO Patent Center and hereby incorporated by reference in its entirety. Said XML copy, created on 1 Aug. 2024, is named 1902004-0002-001-101_SL.xml and is 240,412 bytes in size.

### FIELD OF THE DISCLOSURE

This application relates generally to the field of Bone Morphogenetic Protein (BMP) antagonists, compositions thereof, and methods for use in treating diseases related to BMPs.

### BACKGROUND OF THE DISCLOSURE

The immunopathogenesis of multiple sclerosis (MS) is characterized by pathogenic T- and B-cells that infiltrate the central nervous system and cause multifocal inflammation, demyelination, and neurodegeneration.

Mature oligodendrocytes myelinate axons in the CNS. The development of the myelin sheath is dependent on the proper maturation of oligodendrocytes from precursors cells, a spatially restricted process that is regulated by inductive and repressive cues. Several members of the bone morphogenetic protein family have multiple roles in oligodendrocyte development including in oligodendrocyte precursors where they effect cell lineage decisions (e.g., as repressors by shifting oligodendrocyte precursors into the astrocyte lineage) and by regulation of myelin protein expression in immature oligodendrocytes; in later stages BMPs inhibit cell specialization. For example, BMPs have been shown to block the expression of several myelin structural proteins, preventing myelination. BMPs also inhibit oligodendrocyte development at multiple stages within the lineage and promote astrogenesis. Myelination in the CNS by oligodendrocytes arising from precursors that have progressed through several phenotypic stages assures the rapid and efficient conduction of electrical impulses and promotes neuronal health and survival.

The transforming growth factor-beta (TGF-beta) superfamily contains a variety of growth factors that share common sequence elements and structural motifs. These proteins are known to exert biological effects on a large variety of cell types in both vertebrates and invertebrates. Members of the superfamily perform important functions during embryonic development in pattern formation and tissue specification and can influence a variety of differentiation processes, including adipogenesis, myogenesis, osteogenesis, chondrogenesis, cardiogenesis, hematopoiesis, neurogenesis, and epithelial cell differentiation. The family is divided into two general phylogenetic clades: the more recently evolved members of the superfamily, which includes TGF-betas, Activins, and nodal and the clade of more distantly related proteins of the superfamily, which includes a number of BMPs and GDFs. Hinck (2012) FEBS Letters 586:1860-1870. TGF-beta family members have diverse, often complementary biological effects. By manipulating the activity of a member of the TGF-beta family, it is often possible to cause significant physiological changes in an organism. For example, the Piedmontese and Belgian Blue cattle breeds carry a loss-of-function mutation in the GDF8 (also called myostatin) gene that causes a marked increase in muscle mass. Grobet et al. (1997) Nat Genet., 17(1):71-4. Furthermore, in humans, inactive alleles of GDF8 are associated with increased muscle mass and, reportedly, exceptional strength. Schuelke et al. (2004) N Engl J Med, 350:2682-8. Changes in muscle, bone, fat, red blood cells, and other tissues may be achieved by enhancing or inhibiting signaling (e.g., SMAD 1, 2, 3, 5, and/or 8) that is mediated by ligands of the TGF-beta family. Thus, there is a need for agents that regulate the activity of various ligands of the TGF-beta superfamily, including the bone morphogenetic proteins (BMPs) related to this disclosure.

### SUMMARY OF THE DISCLOSURE

Described herein are polypeptides, compositions, other reagents related to Bone Morphogenetic Protein (BMP) antagonists, and methods for use in treating neurodegenerative and/or demyelinating diseases related to BMP ligand and endogenous signaling. In particular, this disclosure relates to soluble bone morphogenetic protein (BMP) receptor type 1B polypeptides comprising an extracellular domain of ALK6 (activin receptor-like kinase 6) and soluble variants thereof (“ALK6 polypeptides”), which bind BMP ligands and sequester the ligands from binding to their receptors (“BMP antagonists”). The ALK-6 polypeptides of this disclosure bind BMP ligands, including, but not limited to BMP 4 and/or 10. In certain embodiments, ALK-6 polypeptides of this disclosure bind BMP10. In certain embodiments, the ALK6 ECD sequence may comprise one or more point mutations, addition or deletion of c-terminal residues, wherein position 60 is glycine (G) as provided in SEQ ID NO:1.

In embodiments provided herein are soluble recombinant bone morphogenetic protein receptor type-1B fusion proteins comprising an ALK6 ECD polypeptide and a Fc sequence, wherein the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 4; SEQ ID NO: 11, SEQ ID NO: 69; SEQ ID NO: 80 and SEQ ID NO: 113. In certain embodiments, amino acid position 60 is a glycine residue. In certain embodiments, the polypeptide binds BMP10. In certain embodiments, the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from 1, 4, 11-45, 57, 59, 61, 63, 65, 67, 69, 71, 73, 75, 77, 80, 86, 88, 92, 94, 96, 98, 100 and 113.

In other embodiments provided herein are soluble recombinant bone morphogenetic protein receptor type-1B fusion proteins comprising an ALK6 ECD polypeptide and a Fc sequence, wherein the ALK6 ECD polypeptide comprises an amino acid sequence selected from a sequence of SEQ ID NO: 3: Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₂-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Xaa₁₀₀-Xaa₁₀₁-Xaa₁₀₂-Xaa₁₀₃-Xaa₁₀₄-Xaa₁₀₅-Xaa₁₀₆-Xaa₁₀₇-Xaa₁₀₈-Xaa₁₀₉-Xaa₁₁₀-Xaa₁₁₁-Xaa₁₁₂-Xaa₁₁₃;
wherein: Xaa₁₅ is K or P; Xaa₂₀ is F, V, K or Y; Xaa₂₂ is G, H or S; Xaa₂₃ is S, H or G; Xaa₂₇ is E or D; Xaa₂₉ is S or A; Xaa₃₀ is V or I; Xaa₃₃ is I or T; Xaa₃₅ is S or I; Xaa₃₇ is D or N; Xaa₃₉ is Y or H; Xaa₄₂ is T or A; Xaa₄₃ is M or I; Xaa₄₉ is S or Q; Xaa₅₁ is L or E; Xaa₅₂ is P or T; Xaa₅₃ is V or T; Xaa₅₄ is V or L; Xaa₅₅ is T or A; Xaa₅₉ is L or M Xaa₆₀ is G or K; Xaa₆₁ is L or Y; Xaa₆₉ is R or K; Xaa₇₁ is T or S; Xaa₇₃ is I or K; Xaa₇₄ is P or A; Xaa₇₅ is H or Q; Xaa₇₆ is Q or L; Xaa₇₉ is S or T; Xaa₈₄ is T or R; Xaa₈₅ is absent, T or E; Xaa₈₆ is R or N; Xaa₈₇ is N or L; Xaa₈₈ is absent, E or L; Xaa₉₁ is K or Q; Xaa₉₂ is D or L; Xaa₉₄ is H or Q; Xaa₁₀₀ is absent, V or L; Xaa₁₀₁ is absent, V or K; Xaa₁₀₂ is absent, I or N; Xaa₁₀₃ is absent, G or R; Xaa₁₀₄ is absent, P, D or A; Xaa₁₀₅ is absent or F; Xaa₁₀₆ is absent, F or V; Xaa₁₀₇ is absent or D; Xaa₁₀₈ is absent or G; Xaa₁₀₉ is absent, S or P; Xaa₁₁₀ is absent or I; Xaa₁₁₁ is absent, R or H; Xaa₁₁₂ is absent or H; and Xaa₁₁₃ is absent or R.

In certain embodiments, the ALK6 ECD polypeptide comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position.

In further embodiments provided herein are soluble recombinant bone morphogenetic protein receptor type-1B fusion protein comprising an ALK6 ECD polypeptide and a Fc sequence, wherein the ALK6 ECD polypeptide comprises an amino acid sequence selected from a sequence of SEQ ID NO: 5:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀;
wherein Xaa₁₅ is K or P; Xaa₂₀ is F, V, K or Y; Xaa₂₂ is G, H or S; Xaa₂₃ is S, H or G; Xaa₂₇ is E or D; Xaa₂₉ is S or A; Xaa₃₀ is V or I; Xaa₃₃ is I or T; Xaa₃₅ is S or I; Xaa₃₇ is D or N; Xaa₃₉ is Y or H; Xaa₄₂ is T or A; Xaa₄₃ is M or I; Xaa₄₉ is S or Q; Xaa₅₁ is L or E; Xaa₅₂ is P or T; Xaa₅₃ is V or T; Xaa₅₄ is V or L; Xaa₅₅ is T or A; Xaa₅₉ is L or M; Xaa₆₀ is G or K; Xaa₆₁ is L or Y; Xaa₆₉ is R or K; Xaa₇₁ is T or S; Xaa₇₃ is I or K; Xaa₇₄ is P or A; Xaa₇₅ is H or Q; Xaa₇₆ is Q or L; Xaa₇₉ is S or T; Xaa₈₄ is T or R; Xaa₈₅ is absent, T or E; Xaa₈₆ is R or N; Xaa₈₇ is N or L; Xaa₈₈ is absent, E or L; Xaa₉₁ is K or Q; Xaa₉₂ is D or L; and, Xaa₉₄ is H or Q.

In certain embodiments, the ALK6 ECD polypeptide comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, or a combination thereof, wherein _ is a deletion at that position.

In further embodiments provided herein are soluble recombinant bone morphogenetic protein receptor type-1B fusion protein comprising an ALK6 ECD polypeptide and a Fc sequence, wherein the ALK6 ECD polypeptide comprises an amino acid sequence selected from a sequence of SEQ ID NO: 79:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Val₁₀₀-Val₁₀₁-Ile₁₀₂-Gly₁₀₃-Pro₁₀₄-Phe₁₀₅-Phe₁₀₆-Asp₁₀₇-Gly₁₀₈-Ser₁₀₉-Ile₁₁₀-Arg₁₁₁;
wherein Xaa₁₅ is K or P; Xaa₂₀ is F, V, K or Y; Xaa₂₂ is G, H or S; Xaa₂₃ is S, H or G; Xaa₂₇ is E or D; Xaa₂₉ is S or A; Xaa₃₀ is V or I; Xaa₃₃ is I or T; Xaa₃₅ is S or I; Xaa₃₇ is D or N; Xaa₃₉ is Y or H; Xaa₄₂ is T or A; Xaa₄₃ is M or I; Xaa₄₉ is S or Q; Xaa₅₁ is L or E; Xaa₅₂ is P or T; Xaa₅₃ is V or T; Xaa₅₄ is V or L; Xaa₅₅ is T or A; Xaa₅₉ is L or M; Xaa₆₀ is G or K; Xaa₆₁ is L or Y; Xaa₆₉ is R or K; Xaa₇₁ is T or S; Xaa₇₃ is I or K; Xaa₇₄ is P or A; Xaa₇₅ is H or Q; Xaa₇₆ is Q or L; Xaa₇₉ is S or T; Xaa₈₄ is T or R; Xaa₈₅ is absent, T or E; Xaa₈₆ is R or N; Xaa₈₇ is N or L; Xaa₈₈ is absent, E or L; Xaa₉₁ is K or Q; Xaa₉₂ is D or L; and, Xaa₉₄ is H or Q.

In certain embodiments, the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NOs: 15-45, 57, 59, 61, 63, and 65. In certain other embodiments, The fusion protein of claim 7, wherein the ALK6 ECD polypeptide comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position.

In further embodiments provided herein are soluble recombinant bone morphogenetic protein receptor type-1B fusion proteins comprising an ALK6 ECD polypeptide and a Fc sequence, wherein the ALK6 ECD polypeptide comprises an amino acid sequence selected from a sequence of SEQ ID NO: 81:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀-Lys₁₀₁-Asn₁₀₂-Arg₁₀₃-Asp₁₀₄-Phe₁₀₅-Val₁₀₆-Xaa₁₀₇-Xaa₁₀₈-Xaa₁₀₉-Xaa₁₁₀-Xaa₁₁₁-Xaa₁₁₂-Xaa₁₁₃;
wherein Xaa₁₅ is K or P; Xaa₂₀ is F, V, K or Y; Xaa₂₂ is G, H or S; Xaa₂₃ is S, H or G; Xaa₂₇ is E or D; Xaa₂₉ is S or A; Xaa₃₀ is V or I; Xaa₃₃ is I or T; Xaa₃₅ is S or I; Xaa₃₇ is D or N; Xaa₃₉ is Y or H; Xaa₄₂ is T or A; Xaa₄₃ is M or I; Xaa₄₉ is S or Q; Xaa₅₁ is L or E; Xaa₅₂ is P or T; Xaa₅₃ is V or T; Xaa₅₄ is V or L; Xaa₅₅ is T or A; Xaa₅₉ is L or M; Xaa₆₀ is G or K; Xaa₆₁ is L or Y; Xaa₆₉ is R or K; Xaa₇₁ is T or S; Xaa₇₃ is I or K; Xaa₇₄ is P or A; Xaa₇₅ is H or Q; Xaa₇₆ is Q or L; Xaa₇₉ is S or T; Xaa₈₄ is T or R; Xaa₈₅ is absent, T or E; Xaa₈₆ is R or N; Xaa₈₇ is N or L; Xaa₈₈ is absent, E or L; Xaa₉₁ is K or Q; Xaa₉₂ is D or L; Xaa₉₄ is H or Q; Xaa₁₀₇ is absent; Xaa₁₀₈ is absent; Xaa₁₀₉ is absent; Xaa₁₁₀ is absent; Xaa₁₁₁ is absent; Xaa₁₁₂ is absent; and, Xaa₁₁₃ is absent.

In certain embodiments, the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 13. In other embodiments, the ALK6 ECD polypeptide comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, or a combination thereof, wherein _ is a deletion at that position.

In further embodiments provided herein are soluble recombinant bone morphogenetic protein receptor type-1B fusion protein comprising an ALK6 ECD polypeptide and a Fc sequence, wherein the ALK6 ECD polypeptide comprises an amino acid sequence selected from a sequence of SEQ ID NO: 82:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀-Lys₁₀₁-Asn₁₀₂-Arg₁₀₃-Asp₁₀₄-Phe₁₀₅-Val₁₀₆-Xaa₁₀₇-Gly₁₀₈-Xaa₁₀₉-Ile₁₁₀-Xaa₁₁₁-Xaa₁₁₂-Arg₁₁₃;
wherein Xaa₁₅ is K or P; Xaa₂₀ is F, V, K or Y; Xaa₂₂ is G, H or S; Xaa₂₃ is S, H or G; Xaa₂₇ is E or D; Xaa₂₉ is S or A; Xaa₃₀ is V or I; Xaa₃₃ is I or T; Xaa₃₅ is S or I; Xaa₃₇ is D or N; Xaa₃₉ is Y or H; Xaa₄₂ is T or A; Xaa₄₃ is M or I; Xaa₄₉ is S or Q; Xaa₅₁ is L or E; Xaa₅₂ is P or T; Xaa₅₃ is V or T; Xaa₅₄ is V or L; Xaa₅₅ is T or A; Xaa₅₉ is L or M; Xaa₆₀ is G or K; Xaa₆₁ is L or Y; Xaa₆₉ is R or K; Xaa₇₁ is T or S; Xaa₇₃ is I or K; Xaa₇₄ is P or A; Xaa₇₅ is H or Q; Xaa₇₆ is Q or L; Xaa₇₉ is S or T; Xaa₈₄ is T or R; Xaa₈₅ is absent, T or E; Xaa₈₆ is R or N; Xaa₈₇ is N or L; Xaa₈₈ is absent, E or L; Xaa₉₁ is K or Q; Xaa₉₂ is D or L; Xaa₉₄ is H or Q; Xaa₁₀₇ is D or A; Xaa₁₀₉ is P or S; Xaa₁₁₁ is absent; and, Xaa₁₁₂ is absent.

In certain embodiments, the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 67, 73, 75, and 77. In other embodiments, the ALK6 ECD polypeptide comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position.

In further embodiments provided herein are soluble recombinant bone morphogenetic protein receptor type-1B fusion protein comprising an ALK6 ECD polypeptide and a Fc sequence, wherein the ALK6 ECD polypeptide comprises an amino acid sequence selected from a sequence of SEQ ID NO: 83:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀-Lys₁₀₁-Asn₁₀₂-Arg₁₀₃-Xaa₁₀₄-Phe₁₀₅-Val₁₀₆-Xaa₁₀₇-Gly₁₀₈-Xaa₁₀₉-Ile₁₁₀-His₁₁₁-His₁₁₂-Arg₁₁₃;
wherein Xaa₁₅ is K or P; Xaa₂₀ is F, V, K or Y; Xaa₂₂ is G, H or S; Xaa₂₃ is S, H or G; Xaa₂₇ is E or D; Xaa₂₉ is S or A; Xaa₃₀ is V or I; Xaa₃₃ is I or T; Xaa₃₅ is S or I; Xaa₃₇ is D or N; Xaa₃₉ is Y or H; Xaa₄₂ is T or A; Xaa₄₃ is M or I; Xaa₄₉ is S or Q; Xaa₅₁ is L or E; Xaa₅₂ is P or T; Xaa₅₃ is V or T; Xaa₅₄ is V or L; Xaa₅₅ is T or A; Xaa₅₉ is L or M; Xaa₆₀ is G or K; Xaa₆₁ is L or Y; Xaa₆₉ is R or K; Xaa₇₁ is T or S; Xaa₇₃ is I or K; Xaa₇₄ is P or A; Xaa₇₅ is H or Q; Xaa₇₆ is Q or L; Xaa₇₉ is S or T; Xaa₈₄ is T or R; Xaa₈₅ is absent, T or E; Xaa₈₆ is R or N; Xaa₈₇ is N or L; Xaa₈₈ is absent, E or L; Xaa₉₁ is K or Q; Xaa₉₂ is D or L; Xaa₉₄ is H or Q; Xaa₁₀₄ is D or A; Xaa₁₀₇ is absent or D and Xaa₁₀₉ is P or S.

In certain embodiments, the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 12; SEQ ID NO: 14, and SEQ ID NO: 71. In other embodiments, the ALK6 ECD polypeptide comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position.

In certain embodiments, any of the ALK6 fusion proteins may further comprising a linker sequence including, but not limited, to those selected from SEQ ID NO: 6, SEQ ID NO: 7 or SEQ ID NO: 8. In exemplary embodiments, the soluble ALK6 fusion protein comprises an Fc sequence is according to SEQ ID NO: 10; SEQ ID NO: 106 or SEQ ID NO: 107.

In certain embodiments provided herein is a soluble recombinant bone morphogenetic protein receptor type-1B fusion protein comprising: an ALK6 extra cellular domain (ECD) and an Fc domain sequence wherein the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 46-50, 52-56, 58, 60, 62, 64, 66, 68, 70, 72, 74, 76, and 78. In certain other embodiments provided herein is a soluble recombinant bone morphogenetic protein receptor type-1B fusion protein comprising: i) an ALK6 extra cellular domain (ECD) comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence according to SEQ ID NO: 1; ii) a linker selected from SEQ ID NO:6 or SEQ ID NO: 8; and, iii) and Fc sequence.

In certain embodiments provided herein are soluble recombinant bone morphogenetic protein receptor type-1B fusion proteins comprising: an ALK6 extra cellular domain (ECD), a linker sequence and an Fc domain sequence wherein the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 46-50, 52-56, 58, 60, 62, 64, 66, 68, 70, 72, 74, 76, 78, 86, 88, 90, 92, 94, 96, 98, 100, 102, 104, 110 and 113, wherein amino acid position 60 is a glycine residue. In embodiments, the polypeptide binds BMP10. In embodiments, the ALK6 ECD sequence selected from SEQ ID NO: 86, 90, 92, 94, 96, 98, 100, and 102. In certain embodiments, the ALK6 ECD comprises one or more point mutations selected from K20Y, K20F, K20V, H22S, H22G, H23S, and H23G. In further embodiments, the ALK6 ECD comprises an amino acid insertion between position 22 and position 23 of an alanine (A) residue.

Provided herein are soluble recombinant bone morphogenetic protein receptor type-1B fusion proteins comprising: an ALK6 extra cellular domain (ECD), a linker sequence and an Fc domain sequence wherein the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 86 or 90, wherein amino acid position 60 is a glycine (G) residue. In embodiments, the ALK6 ECD sequence comprises at least one point mutation as compared to SEQ ID NO: 1. In embodiments, the point mutation is selected from K20Y, K20F, K20V, H22S, H22G, H23S, and H23G. In embodiments, the polypeptide binds BMP10.

In certain embodiments provided herein are soluble recombinant bone morphogenetic protein receptor type-1B fusion proteins comprising: an ALK6 extra cellular domain (ECD), a linker sequence and an Fc domain sequence wherein the fusion protein sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical SEQ ID NO: 87 and SEQ ID NO: 91.

Provided herein are pharmaceutical composition comprising an ALK6 fusion protein of this disclosure; and, at least one pharmaceutical acceptable carrier or buffer.

In certain embodiments provided herein are methods for treating neurodegenerative disorders, comprising administering an effective amount of a pharmaceutical composition comprising an ALK6 fusion protein of this disclosure to a subject in need thereof. In embodiments, the neurodegenerative disorder is Alzheimer's disease, Parkinson's disease, or Amyotrophic lateral sclerosis (ALS).

In embodiments the neurodegenerative disorder is ALS wherein the treating comprises slowing progression of amyotrophic lateral sclerosis, reducing intensity of symptoms associated with amyotrophic lateral sclerosis, reducing onset of symptoms associated with amyotrophic lateral sclerosis, reducing weight loss associated with amyotrophic lateral sclerosis, reversing weight loss associated with amyotrophic lateral sclerosis, delaying mortality associated with amyotrophic lateral sclerosis, and combinations thereof. In embodiments, the subject exhibits a greater than 20% improvement in ALS Functional Rating Scale, Revised (ALSFRS-R) score when compared to baseline; the patient exhibits a greater than 30% improvement in ALS Functional Rating Scale, Revised (ALSFRS-R) score when compared to baseline; and/or the improvement is apparent in a time period selected from the group consisting of less than about 9 months, less than about 6 months, less than about 3 months, and less than about 1 month.

In other embodiments provided herein are methods for treating a demyelinating disease, comprising administering an effective amount of a pharmaceutical composition comprising an ALK6 fusion protein of this disclosure to a subject in need thereof. In embodiments, the demyelinating disease is multiple sclerosis, acute-disseminated encephalomyelitis (ADEM) or acute haemorrhagic leucoencephalitis (AHL).

In embodiments the demyelinating disease is multiple sclerosis, wherein the treating is effective to alleviate a symptom of or a condition associated with multiple sclerosis; wherein the treating is effective to reduce relapse rate, reduce rate of confirmed relapses requiring hospitalization and/or IV steroids, reduce the accumulation of disability, reduce or inhibit progression of the level of fatigue, improve or inhibit deterioration of the functional status, improve or inhibit deterioration of the general health, reduce MRI-monitored disease activity, decrease the hazard ratio for relapse-free survival or reduce cognitive impairment in the human patient; and/or wherein the treatment is effective to decrease the hazard ratio for time to confirmed relapse in the human patient; wherein the treatment is effective to reduce the annualized relapse rate in the human patient.

In certain embodiments provided herein is method for treating neurodegenerative disorders, comprising: administering an effective amount of a pharmaceutical composition to a subject in need thereof, wherein the composition comprises a soluble recombinant bone morphogenetic protein receptor type-1B fusion protein comprising an ALK6 ECD polypeptide and a Fc sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to SEQ ID NO: 1.

In certain other embodiments provided herein is a method for treating a demyelinating disease, comprising: administering an effective amount of a pharmaceutical composition to a subject in need thereof, wherein the composition comprises a soluble recombinant bone morphogenetic protein receptor type-1B fusion protein comprising an ALK6 ECD polypeptide and a Fc sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to SEQ ID NO: 1.

In certain embodiments provided herein is a method of treating multiple sclerosis, comprising administering an effective amount of a pharmaceutical composition to a subject in need thereof, wherein the composition comprises a soluble recombinant bone morphogenetic protein receptor type-1B fusion protein comprising an ALK6 ECD polypeptide, a linker and a Fc sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from SEQ ID NO: 1, 4, 11-45, 57, 59, 61, 63, 65, 67, 69, 71, 73, 75, 77, 80, 86, 88, 90, 92, 94, 96, 98, 100, 102 and 113, wherein amino acid position 60 is a glycine (G) residue. In certain embodiments, the ALK6 ECD sequence selected from SEQ ID NO: 86, 90, 92, 94, 96, 98, 100, and 102. In certain embodiments, the ALK6 ECD sequence comprises at least one point mutation as compared to SEQ ID NO: 1. In certain embodiments, the point mutation is selected from K20Y, K20F, K20V, H22S, H22G, H23S, and H23G. In embodiments, the soluble recombinant bone morphogenetic protein receptor type-1B fusion protein for treating MS binds BMP10. In embodiments, the ALK6 ECD comprises an amino acid insertion between position 22 and position 23 of an alanine (A) residue.

In certain embodiments provided herein is a method of treating multiple sclerosis, comprising administering an effective amount of a pharmaceutical composition to a subject in need thereof, wherein the composition comprises a soluble recombinant bone morphogenetic protein receptor type-1B fusion protein wherein the protein comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical SEQ ID NO: 87 and SEQ ID NO: 91.

### BRIEF DESCRIPTION OF THE DRAWINGS

FIGS. 1A and 1B show SDS-PAGE gels for a number soluble ALK6 Fc fusion proteins purified using protein A chromatography (SEQ ID NO: 46-50, 70, 72, 74, 76, 84 and 85) and the cleavage fragment indicating a protease cleavage site present in SEQ ID NO: 49, 47 and 76. See Example 1.

FIGS. 2A and 2B show SDS-PAGE gels for a number of soluble ALK6 Fc fusion protein variants using purified using protein A chromatography and demonstrating no cleavage fragments and indicating removal of the protease cleavage site and reduced aggregation for some variants in the non-reduced lanes of the gel. FIG. 2A shows the quantity of soluble ALK6 Fc fusion protein generated in 200 ml of cell culture demonstrating significantly improved expression for some of the variant (e.g. SEQ ID NO: 60 and 62). Those variants have point mutations replacing two histidine residues (aa22 and aa23) with serine and glycine, (SEQ ID NO: 60 also has the lysine at position 20 replaced with a tyrosine) and when compared to the expression of SEQ ID NO: 46, indicates doing so provides dramatic improvement in the expression level of those fusion proteins. See Example 2.

FIG. 3A shows BMP4 and BMP10 binding for soluble ALK6 Fc fusion proteins of this disclosure. The binding data demonstrates two of the variants (SEQ ID NO: 46 and 52) retain BMP4 and BMP10 ligand binding (as compared to the ALK6 wt sequence, which binds BMP2, BMP4, BMP6, BMP7, BMP10 and GDF5, GDF6 and GDF7), but that SEQ ID NO: 55 no longer binds BMP10 indicating the LGL to MKY change at positions 59, 60 and 61, respectively, is responsible for the loss of binding. See Example 3.

FIG. 3B shows ligand binding for soluble ALK6 Fc fusion proteins of this disclosure, wherein NB indicates no binding and a blank box in the table means the ligand binding was not tested. SEQ ID NO: 89 demonstrated a loss of BMP10 binding (as compared to ALK6 wt sequence) and which contains the same G60K point mutation as SEQ ID NO: 55 (which also has a loss of BMP10 binding and a reduction in BMP7 binding) indicating that point mutation is responsible for the change in ligand binding for BMP10. SEQ ID NO: 89 (and SEQ ID NO: 91 and 93) also demonstrates a retention of BMP7 binding (as compared to ALK6 wt sequence), while SEQ ID NO: 55 only demonstrates weak binding to BMP7. See Example 3.

FIG. 4 shows an IC50 test of SEQ ID NO: 87 in a BMP Responsive Luciferase Assay for the BMP2, BMP4, BMP6 and BMP10 ligands. See Example 4.

FIG. 5 shows an IC50 test of SEQ ID NO: 70 in a BMP Responsive Luciferase Assay for the BMP2, BMP4, BMP6 and BMP10 ligands. See Example 4.

FIG. 6A shows treatment with SEQ ID NO: 46 (10 mg/ml) in a PLP experimental autoimmune encephalomyelitis (EAE) mouse model demonstrating a reduction in disease severity as measured in a clinical score and comparable with Gilenya® (a treatment for relapsing forms of multiple sclerosis (MS)). See Example 5.

FIG. 6B shows treatment with SEQ ID NO: 70 (10 mg/kg), SEQ ID NO: 87 (30 mg/kg) and SEQ ID NO: 131 (30 mg/kg) dosed on day three (3) in a PLP experimental autoimmune encephalomyelitis (EAE) mouse model demonstrating a reduction in disease severity as measured in a clinical score, with SEQ ID NO: 87 performing slightly better than SEQ ID NO: 70 up to about day 23 and then SEQ ID NO: 87 demonstrated a marked improvement in the clinical score as compared to SEQ ID NO: 70. SEQ ID NO: 131, which is an ALK3 wildtype (wt) sequence fused to an Fe sequence, performed no better than vehicle. ALK3 wt binds to BMP2, BMP4, BMP6, BMP7, but not BMP10, consistent with the theory that BMP10 binding is an important factor in performance of the ALK6 fusion proteins of this disclosure for reducing disease severity in the EAE murine model, and as a treatment for multiple sclerosis (MS). See Example 5.

FIG. 7 shows treatment with SEQ ID NO: 87; SEQ ID NO: 89 and SEQ ID NO: 91 in a PLP experimental autoimmune encephalomyelitis (EAE) mouse model demonstrating a reduction in disease severity as measured in a clinical score. See Example 5. SEQ ID NO: 87 and SEQ ID NO: 91 demonstrated similar performance for reducing disease severity as measured by clinical score. SEQ ID NO: 89, which showed a loss of BMP10 binding, but retained BMP7 binding, (See FIG. 3B) performed slightly better than vehicle. Without wishing to be bound by a theory, it appears that maintaining BMP10 binding is an important factor in performance of the ALK6 fusion proteins of this disclosure for reducing disease severity in the EAE murine model, and as a treatment for multiple sclerosis (MS). That is further supported by SEQ ID NO: 131, which showed no reduction in clinical score. See FIG. 6B.

FIG. 8 shows treatment with SEQ ID NO: 87 (20 mg/kg) and SEQ ID NO: 46 (10 mg/kg) in a MOG experimental autoimmune encephalomyelitis (EAE) mouse model demonstrating a reduction in disease severity as measured in a clinical score, consistent with the data in the PLP-EAE model. See Example 6.

FIG. 9 shows paw weight from a DTH experiment, wherein mice were immunized with BSA and then treated with SEQ ID NO: 87, SEQ ID NO: 46, dexamethasone or Gilenya®. Both dexamethasone and Gilenya® are known immunosuppressive therapeutics of the adaptive immune response. Consistent with that, dexamethasone and Gilenya® treated mice demonstrated a paw weight consistent with the no DTH mice group. Alternatively, ALK6 variant fusion proteins of this disclosure (e.g. SEQ ID NO: 46 and SEQ ID NO: 87) demonstrated an increase in paw weight comparable to vehicle indicating SEQ ID NO: 87 and SEQ ID NO: 46 do not suppress the adaptive immune response. See Example 10. In other words, the ALK6 variant fusion proteins of this disclosure are not immunosuppressive agents of the adaptive immune system, and while some of the variants are potent inhibitors of disease severity in a murine model for MS, they do not appear to be suppressing the adaptive immune system in the CNS to do so. Currently available therapeutics for MS (e.g., Gilenya®) mechanistically, at least in part, inhibit the adaptive immune response in the brain providing relief from one or more symptoms of MS.

FIG. 10 shows paw thickness from a DTH experiment wherein mice were immunized with BSA and then treated with SEQ ID NO: 87, SEQ ID NO: 46, dexamethasone or Gilenya®. The mice treated with the immunosuppressive drugs, dexamethasone and Gilenya®, demonstrated a paw thickness consistent with the no DTH mice group. Alternatively, ALK6 variant fusion proteins of this disclosure (e.g. SEQ ID NO: 46 and SEQ ID NO: 87) demonstrated an increase in paw thickness comparable to vehicle, indicating SEQ ID NO: 87 and SEQ ID NO: 46 do not suppress the adaptive immune response. See Example 10. Provided herein is a method for Provided herein is a method for treating multiple sclerosis comprising administering an effective amount of the soluble ALK6 Fc fusion polypeptide of this disclosure to a subject in need thereof, wherein the efficacy of the ALK6 Fc fusion polypeptide is likely not due to a suppression of the adaptive immune response similar to other approved immunosuppressive therapies.

### DETAILED DESCRIPTION OF THE DISCLOSURE

### Overview

This disclosure relates to soluble bone morphogenetic protein (BMP) receptor type 1B polypeptides comprising an extracellular domain of ALK6 (activin receptor-like kinase 6) and variants thereof (“ALK6 polypeptides”), soluble polypeptides, dimeric complexes, methods of making and uses thereof. Endogenous ALK6 is a member of the TGF-beta superfamily wherein signaling in vivo is mediated by heteromeric complexes of type II and type II serine/threonine kinase receptors, which phosphorylate and activate downstream SMAD proteins (e.g., SMAD proteins 1, 2, 3, 5, and 8) upon ligand stimulation. See, e.g. Massague (2000) Nat. Rev, Mol. Cell Biol. 1:169-178, These type I and type II receptors are transmembrane proteins, composed of a ligand-binding extracellular domain (ECD) with cysteine-rich region, a transmembrane domain, and a cytoplasmic domain with predicted serine/threonine kinase specificity. In general, type I receptors mediate intracellular signaling while the type 1 receptors are required for binding TGF-beta superfamily ligands. Type I and II receptors form a stable complex after ligand binding, resulting in phosphorylation of type I receptors by type II receptors.

Ligands for ALK6 are BMPs which are members of the TGF-beta superfamily, and exhibit broad spectra of biological activities in various tissues, including bone, cartilage, blood vessels, heart, kidney, neurons, liver and lung. The BMPs and GDFs together form a family of cysteine-knot cytokines sharing the characteristic fold of the TGF-beta superfamily. See, e.g., Rider et al. (2010) Biochem J., 429(1):1-12. This family includes, for example, BMP2, BMP4, BMP5, BMP6, BMP7, BMP2a, BMP3, BMP3b (also known as GDF10), BMP8, BMP8a, BMP8b, BMP9 (also known as GDF2). BMP10, BMP11 (also known as GDF10, BMP12 (also known as GDF7), BMP13 (also known as GDF6), BMP14 (also known as GDF5), BMP15, GDF1, GDF3 (also known as VGR2), GDF8 (also known as myostatin). GDF9, and GDF15. Besides the ability to induce bone formation, which gave the BMPs their name, the BMP/GDFs display morphogenetic activities in the development of a wide range of tissues, BMP/GDF homo- and hetero-dimers interact with combinations of type j and type 11 receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines.

The transforming growth factor (TGF-3) superfamily signaling pathways are known to impact synaptogenesis, axonal and dendritic growth, synaptic transmission, neurogenesis and neuronal survival. This pathway can lose its physiologic regulation in pathologic conditions, leading to in some circumstances neurodegenerative diseases and demyelinating diseases. Moreover, ALK-6 shows a restricted expression pattern during mouse embryo development and in adult tissue is primarily expressed in brain and lung tissue (Dewulf et al. Endocrinology (1995) June; 136(6):2652-63) and are abundantly expressed in human astrocytes (a subtype of glial cells that make up the majority of cells in the human central nervous system (CNS)) and to a lesser extent, on oligodendrocytes.

Astrocytes are a population of cells with distinctive morphological and functional characteristics that differ within specific areas of the brain. Postnatally, astrocyte progenitors migrate to reach their brain area and related properties. They have a regulatory role of brain functions that are implicated in neurogenesis and synaptogenesis, controlling blood-brain barrier permeability and maintaining extracellular homeostasis. Mature astrocytes also express some genes enriched in cell progenitors, suggesting they can retain proliferative potential. Considering heterogeneity of cell population, it is not surprising that their disorders are related to a wide range of different neuro-pathologies. Brain diseases are characterized by the active inflammatory state of the astrocytes. In particular, the loss of astrocytes function as a result of cellular senescence could have implications for the neurodegenerative disorders, such as Alzheimer disease and Huntington disease, and for the aging brain. Dysregulation of astrocytes, through binding of their BMP ligands, contribute to the loss of neuroprotection and to the gaining of pathological characteristics.

Oligodendrocytes are the myelinating cells of the central nervous system (CNS). They are the end product of a cell lineage which has to undergo a complex and precisely timed program of proliferation, migration, differentiation, and myelination to finally produce the insulating sheath of axons. Due to this complex differentiation program, and due to their unique metabolism/physiology, oligodendrocytes count among the most vulnerable cells of the CNS. Remyelination, the restoration of new myelin sheaths to demyelinated axons, is not performed by pre-existing mature oligodendrocytes but involves in most cases the generation of new mature oligodendrocytes from the adult, quiescent oligodendrocyte progenitor/precursor cell (OPC) pool distributed throughout the CNS. OPCs express bone morphogenetic protein (BMP) receptor type 1B (ALK6), wherein the BMP ligands BMP-2 and 4 promote dose-dependent astrocyte differentiation of adult OPCs with concurrent suppression of remyelinating oligodendrocytes (OL) differentiation. (Cheng et al. Stem Cells (2007) December; 25(12):3204-3214).

The process of remyelination in adults, with suppressed or inactive BMP signaling via ALK6 allowing for oligodendrocyte maturation, takes place in several different steps. First, local adult OPCs must switch from an essentially quiescent state to a regenerative phenotype. This transition can be triggered by factors derived from activated microglia cells and astrocytes, and not by the demyelination per se, and leads to OPC proliferation and recruitment to demyelinated areas. Then, the differentiation of OPCs to remyelinating oligodendrocytes (OL) starts. Remyelination has been extensively studied in MS. In accordance with the basic concepts described above, the recruitment of OPCs and early remyelination is extensive in very early stages of demyelination, in lesions which are still infiltrated by macrophages and lymphocytes, and in plaques formed at early stages of the disease. Remyelination largely fails at the later (progressive) stage of the disease (Brandl and Lassmann; Acta Neuropathol (2010) 119(1):37-53).

Provided herein in embodiments are BMP trap molecules (“BMP antagonists”) comprising the soluble ALK6 polypeptides of this disclosure, which bind BMP ligands and prevent their binding to endogenous transmembrane receptors thus down regulating the BMP signaling pathway. This down regulation inhibits, for example, the Smad1/5/8 cascade alleviating certain neurological diseases such as neurodegenerative and/or de-myelinating diseases (e.g., multiple sclerosis, Alzheimer's disease and ALS). As used herein, a “trap molecule” sequesters the respective ligand (e.g. BMPs) before it can interact with the endogenous receptor and thus inhibiting the signal transduction pathway. The soluble ALK6 polypeptides, when fused to a heterologous domain (e.g., Fc domain) (“fusion polypeptide”) form homodimers.

Provided in certain embodiments are methods for treating a demyelinating disease, comprising administering the soluble ALK6 polypeptides of this disclosure to a subject in need thereof. In certain embodiments, the demyelinating disease is an autoimmune disease such as multiple sclerosis. In certain other embodiments, the demyelinating disease is acute-disseminated encephalomyelitis (ADEM) or acute haemorrhagic leucoencephalitis (AHL). In certain embodiments the method for treating multiple sclerosis comprises administering a pharmaceutical composition to a subject in need thereof, wherein the composition comprises a soluble recombinant bone morphogenetic protein receptor type-1B fusion protein comprising an ALK6 ECD polypeptide and a Fc sequence of this disclosure.

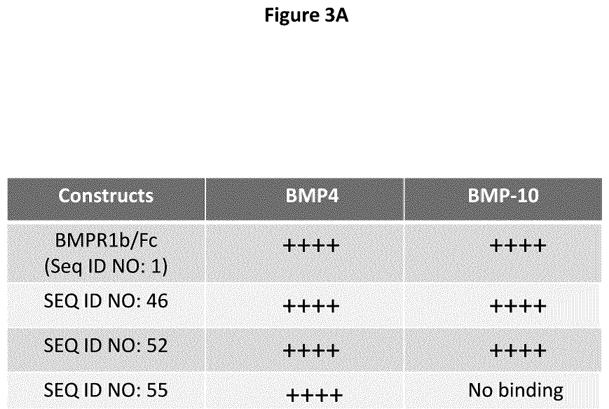

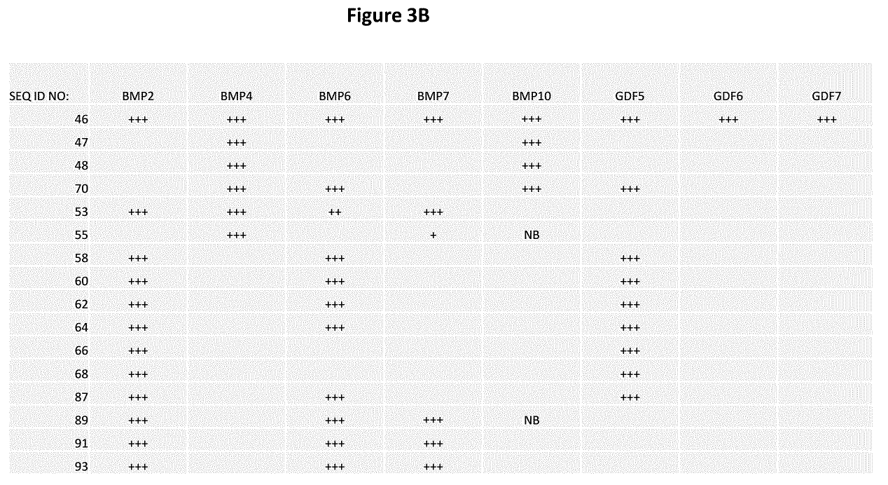

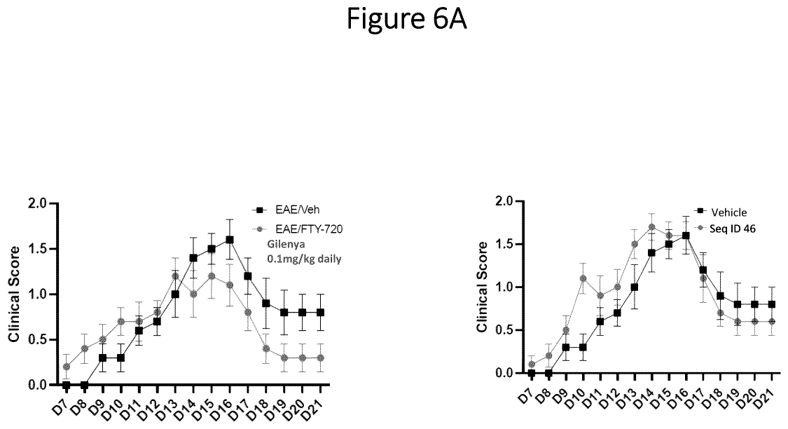

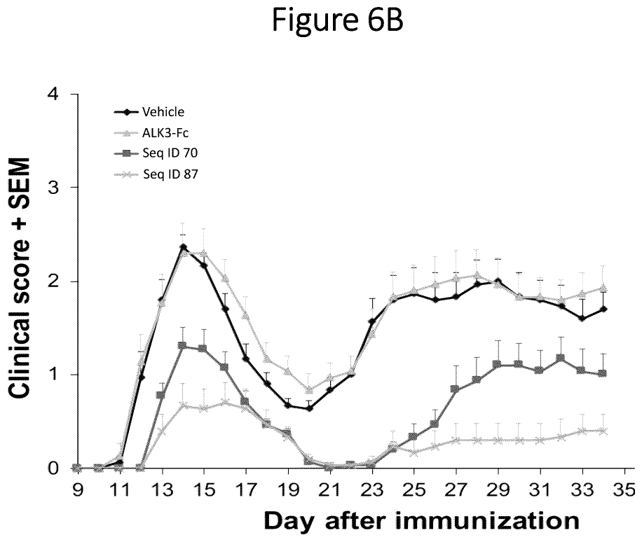

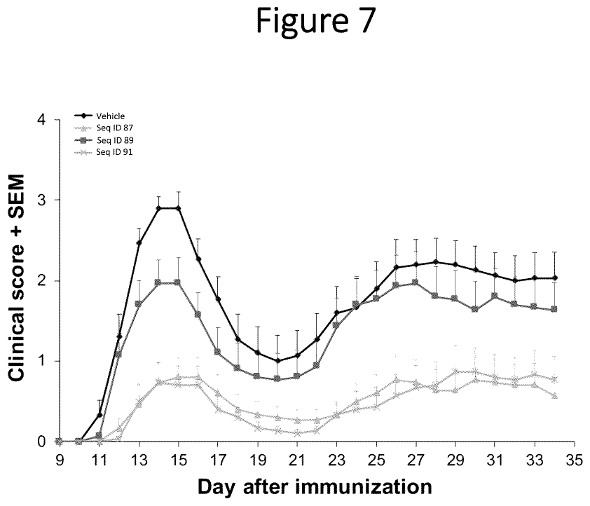

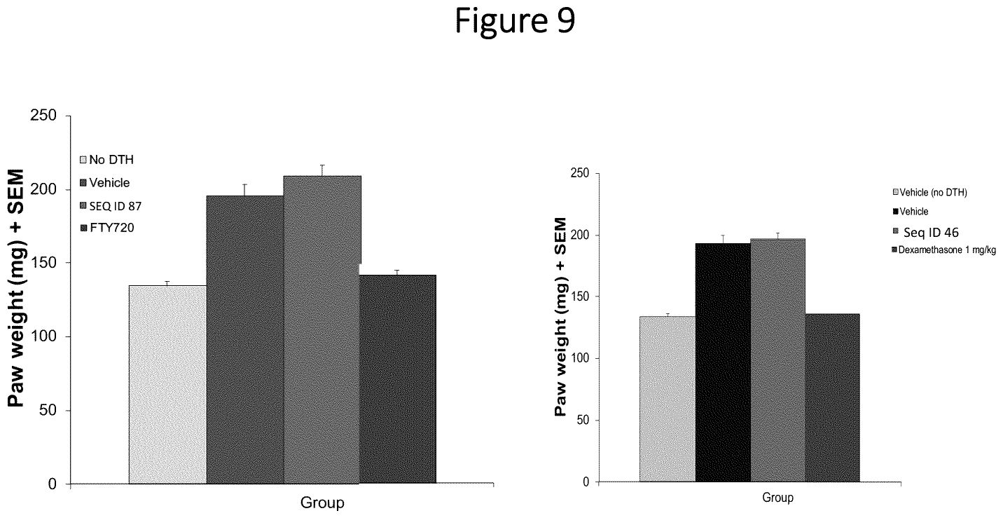

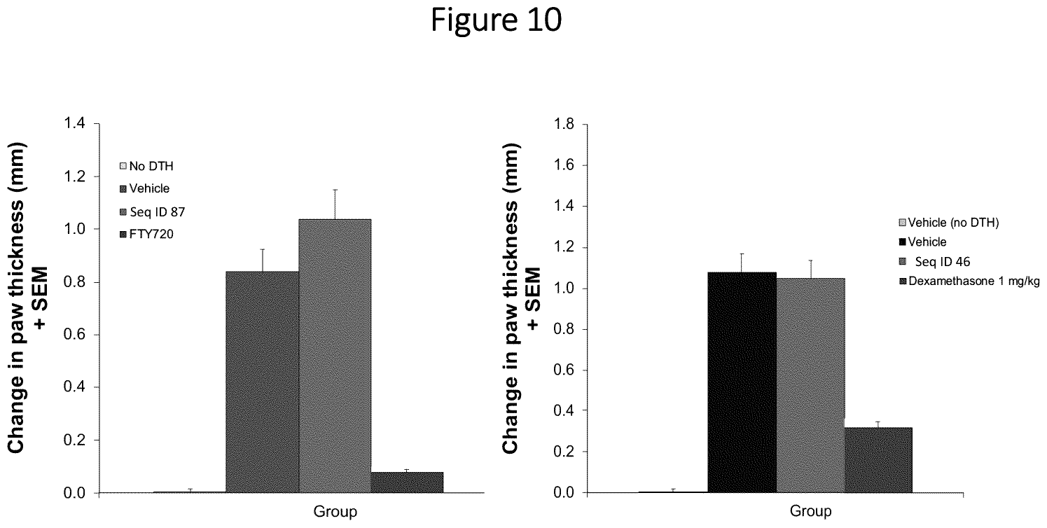

Applicants surprising found that maintaining a glycine (G) residue at position 60, according to SEQ ID NO: 1, provides an ALK6 ECD variant that binds BMP10 (even with one or more point mutations or a deletion of C-terminal residues). ALK6 ECD variants, such as SEQ ID NO: 55 and SEQ ID NO: 89, that had a point mutation at that position (e.g., G60K) lost BMP10 binding as compared to the ALK6 wt ECD sequence (SEQ ID NO: 46). See FIG. 3B. Testing of those ALK6 ECD variants in an experimental autoimmune encephalomyelitis (EAE) mouse model (for multiple sclerosis) demonstrated the importance of BMP10 binding for reducing the severity of disease. See FIG. 6B, which provides a comparison to ALK3 that does not bind BMP10 (and performs no better than vehicle in vivo), and FIG. 7, which provides a comparison to SEQ ID NO: 89 (loss of BMP10 binding) and performs no better than vehicle in vivo. Moreover, the ALK6 ECD fusion proteins of this disclosure were also tested in a delayed type hypersensitivity (DTH) assay to ascertain the immunosuppressive properties of the ALK6 ECD variant polypeptides. Surprisingly, the ALK6 wt ECD sequence (SEQ ID NO: 46) and the variant test (SEQ ID NO: 87) do not suppress the adaptive immune response. See FIGS. 9 and 10. In other words, the ALK6 ECD fusion proteins of this disclosure are not immunosuppressive therapeutic agents of the adaptive immune response. That is in contrast to other marketed therapies for multiple sclerosis (MS) such as Gilenya®, a known immunosuppressive agent. Accordingly, provided herein are soluble recombinant bone morphogenetic protein receptor type-1B fusion proteins comprising an ALK6 extra cellular domain (ECD) polypeptide according to this disclosure, a linker sequence and a Fc sequence, which bind BMP10 and are not immunosuppressive of the adaptive immune response, for use in treating multiple sclerosis.

Provided in certain embodiments are methods for treating a neurodegenerative disorder, comprising administering the soluble ALK6 polypeptides of this disclosure to a subject in need thereof. In embodiments, the neurodegenerative disorder is Alzheimer's disease, Parkinson's disease, or Amyotrophic lateral sclerosis (ALS). In certain embodiments the neurodegenerative disorder is ALS. In certain embodiments the method for treating the neurodegenerative disorder comprises administering a pharmaceutical composition to a subject in need thereof, wherein the composition comprises a soluble recombinant bone morphogenetic protein receptor type-1B fusion protein comprising an ALK6 ECD polypeptide and a Fc sequence of this disclosure.

### Definitions

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity. As used herein the ALK6 polypeptides are referred to as antagonists for the endogenous ALK6 transmembrane receptor signaling pathway wherein the ALK6 polypeptides bind and sequester their endogenous BMP ligand.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤5-fold and more preferably ≤2-fold of a given value.

Numeric ranges are inclusive of the numbers defining the range. Measured and measurable values are understood to be approximate, taking into account significant digits and the error associated with the measurement. Accordingly, unless indicated to the contrary, the numerical parameters set forth in the following description and appended claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “or” is used herein in the inclusive sense, i.e., equivalent to “and/or” unless the context clearly requires otherwise.

The use of “comprise”, “comprises”, “comprising”, “contain”, “contains”, “containing”, “include”, “includes”, and “including” is not intended to be limiting, and means that at least the named compound, element, particle, or method step is present in the composition or article or method, but does not exclude the presence of other compounds, materials, particles, method steps, even if the other such compounds, material, particles, method steps have the same function as what is named.

The terms “treatment”, “treating”, “alleviating” and the like are used herein to generally mean obtaining a desired pharmacologic and/or physiologic effect, and may also be used to refer to improving, alleviating, and/or decreasing the severity of one or more clinical complication of a condition being treated. The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure is achieved by administering one or more soluble ALK6 polypeptides of the present disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

Also, in describing the embodiments, terminology will be resorted to for the sake of clarity. It is intended that each term contemplates its broadest meaning as understood by those skilled in the art and includes all technical equivalents which operate in a similar manner to accomplish a similar purpose.

### Bone Morphogenetic Protein (BMP) Receptor Type 1B Polypeptides

The present disclosure generally relates to soluble ALK6 polypeptides, variants thereof, compositions, and uses thereof (e.g., treating, preventing or reducing the progression rate and/or severity of neurological disorders (e.g., neurodegenerative and demyelinating disorders)). Such neurological disorders include, but not limited to, multiple sclerosis and ALS. As used herein the term “ALK6 polypeptide” refers to the extracellular domain of human ALK6 provided for example in SEQ ID NO: 1, as well as any variants thereof (including mutants, fragments, fusions, and peptidomimetic forms) that retain a useful activity.

Numbering of amino acids for all ALK6-related polypeptides described herein is based on the numbering of the human ALK6 processed (mature) extracellular domain (ECD) human protein sequence provided below (SEQ ID NO: 1), unless specifically designated otherwise.

(SEQ ID NO: 1)
KKEDGESTAPTPRPKVLRCKCHHHCPEDSVNNICSTDGYCFTMIEEDDS
GLPVVTSGCLGLEGSDFQCRDTPIPHQRRSIECCTERNECNKDLHPTLP
PLKNRDFVDGPIHHR

The C-terminal “tail” of the extracellular domain is indicated by single underline. In certain embodiments the ALK6 ECD sequence with the “tail” deleted (a Δ14 sequence or Δ13 sequence) (“SEQ ID NO: 4 variant”) is as follows:

(SEQ ID NO: 4)
KKEDGESTAPTPRPKVLRCKCHHHCPEDSVNNICSTDGYCFTMIEEDDS
GLPVVTSGCLGLEGSDFQCRDTPIPHQRRSIECCTERNECNKDLHPTLP
P(L)

In certain embodiments the 14 amino acid deleted C terminal tail sequence is replaced with VVIGPFFDGSIR (SEQ ID NO: 2) providing a variant ALK6 ECD polypeptide (“SEQ ID NO: 11 variant”) as follows:

(SEQ ID NO: 11)
KKEDGESTAPTPRPKVLRCKCHHHCPEDSVNNICSTDGYCFTMIEEDDS
GLPVVTSGCLGLEGSDFQCRDTPIPHQRRSIECCTERNECNKDLHPTLP
PVVIGPFFDGSIR

In certain other embodiments, a seven amino acid portion of the “tail” sequence is deleted (a Δ7 sequence) from SEQ ID NO: 1 (“SEQ ID NO: 69 variant”), providing the following sequence:

(SEQ ID NO: 69)
KKEDGESTAPTPRPKVLRCKCHHHCPEDSVNNICSTDGYCFTMIEEDDS
GLPVVTSGCLGLEGSDFQCRDTPIPHQRRSIECCTERNECNKDLHPTLP
PLKNRDEV

In certain other embodiments provided herein the last two histidine amino acids of the “tail” sequence from SEQ ID NO: 1 (“SEQ ID NO: 80 variant”) are deleted providing an ALK6 ECD polypeptide having the sequence:

(SEQ ID NO: 80)
KKEDGESTAPTPRPKVLRCKCHHHCPEDSVNNICSTDGYCFTMIEEDDSGLPVVTSGCL
GLEGSDFQCRDTPIPHQRRSIECCTERNECNKDLHPTLPPLKNRDFVDGPI__R

In certain other embodiments, a seven amino acid portion of the “tail” sequence is deleted (a Δ7 sequence) from SEQ ID NO: 1 (“SEQ ID NO: 113 variant”), and an arginine amino acid added between aa22 and aa23, providing the following sequence:

(SEQ ID NO: 113)
KKEDGESTAPTPRPKVLRCKCHAHHCPEDSVNNICSTDGYCFTMIEEDDSGLPVVTSGC
LGLEGSDFQCRDTPIPHQRRSIECCTERNECNKDLHPTLPPLKNRDFV_______

In certain embodiments, the ALK6 polypeptide comprises a combination of a modified version of SEQ ID NO: 1 (i.e., the extracellular domain of “wild-type” or naturally occurring BMP antagonist). In embodiments, the wild-type ALK-6 does not include the C-terminal sequence of SEQ ID NO: 1, such as that shown in SEQ ID NO: 4. In embodiments, the ALK6 polypeptide comprises a modified version of SEQ ID NO: 1 wherein a seven amino acid portion of the “tail” sequence is deleted (SEQ ID NO: 69) or wherein two histidine amino acids at positions 111 and 112 of the ALK6 polypeptide ECD sequence are deleted (SEQ ID NO: 80). In certain embodiments, the ALK6 polypeptide comprises SEQ ID NO: 11 wherein the fourteen (14) amino acid “tail” deleted sequence is replaced with SEQ ID NO: 2. As described in more detail below, any of these sequences (e.g., SEQ ID NO: 1, 4, 11, 69, 80 or 113) can be modified to alter and/or optimize ligand binding and/or to remove undesirable properties (e.g., protease cleave site(s)).

Accordingly, a general formula for an active portion (e.g., ligand binding) of ALK6 is a polypeptide that comprises, consists essentially of, or consists of SEQ ID NO: 1. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 ECD of SEQ ID NO: 1.

In embodiments, an active portion (e.g., ligand binding) of ALK6 is a polypeptide that comprises, consists essentially of, or consists of SEQ ID NO: 4. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 4.

In embodiments, an active portion (e.g., ligand binding) of ALK6 is a polypeptide that comprises, consists essentially of, or consists of SEQ ID NO: 11. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 11.

In embodiments, an active portion (e.g., ligand binding) of ALK6 is a polypeptide that comprises, consists essentially of, or consists of SEQ ID NO: 69. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 69.

In embodiments, an active portion (e.g., ligand binding) of ALK6 is a polypeptide that comprises, consists essentially of, or consists of SEQ ID NO: 80. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 80.

In embodiments, an active portion (e.g., ligand binding) of ALK6 is a polypeptide that comprises, consists essentially of, or consists of SEQ ID NO: 113. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 113

Provided herein are specific point mutations or point deletions within any of the above disclosed SEQ ID NO: 1, 4, 11, 69, 80 and 113. Those exemplified mutations and/or deletions and/or addition are represented in SEQ ID NO: 3, 5, 79, 81, 82, 83, 108, and 109. Provided herein are ALK6 ECD sequence selected from any sequence according to SEQ ID NO: 3, 5, 79, 81, 82, 83, 108 and 109.

In certain embodiments, the soluble ALK6 polypeptide comprises an ECD sequence of ALK6 according to SEQ ID NO: 3. In certain embodiments, the soluble ALK6 polypeptide comprises an ECD sequence of ALK6 according to SEQ ID NO: 5. In certain embodiments, the soluble ALK6 polypeptide comprises an ECD sequence of ALK6 according to SEQ ID NO: 79. In certain embodiments, the soluble ALK6 polypeptide comprises an ECD sequence of ALK6 according to SEQ ID NO: 81. In certain embodiments, the soluble ALK6 polypeptide comprises an ECD sequence of ALK6 according to SEQ ID NO: 82. In certain embodiments, the soluble ALK6 polypeptide comprises an ECD sequence of ALK6 according to SEQ ID NO: 83. In certain embodiments, the soluble ALK6 polypeptide comprises an ECD sequence of ALK6 according to SEQ ID NO: 108. In certain embodiments, the soluble ALK6 polypeptide comprises an ECD sequence of ALK6 according to SEQ ID NO: 109.

In certain embodiments, the soluble ALK-6 polypeptide comprises an active portion (e.g., ligand binding) of ALK6 according to SEQ ID NO: 3 as shown below using standard three-letter amino acid abbreviations:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₈-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₈-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₈-Thr₉₆-Leu₉₇-Pro₉₈-Pro₉₉-Xaa₁₀₀-Xaa₁₀₁-Xaa₁₀₂-Xaa₁₀₃-Xaa₁₀₄-Xaa₁₀₅-Xaa₁₀₆-Xaa₁₀₇-Xaa₁₀₈-Xaa₁₀₉-Xaa₁₁₀-Xaa₁₁₁-Xaa₁₁₂-Xaa₁₁₃;
wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₀ is absent, V or L;
Xaa₁₀₁ is absent, V or K;
Xaa₁₀₂ is absent, I or N;
Xaa₁₀₃ is absent, G or R;
Xaa₁₀₄ is absent, P, D or A;
Xaa₁₀₅ is absent or F;
Xaa₁₀₆ is absent, F or V;
Xaa₁₀₇ is absent or D;
Xaa₁₀₈ is absent or G;
Xaa₁₀₉ is absent, S or P;
Xaa₁₁₀ is absent or I;
Xaa₁₁₁ is absent, R or H;
Xaa₁₁₂ is absent or H; and,
Xaa₁₁₃ is absent or R (SEQ ID NO: 3).

In embodiments, the ALK6 ECD polypeptide of the fusion protein comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position. In certain embodiments, the ALK6 ECD polypeptide of the fusion protein comprises one or more substitutions selected from L59M, G60K, and L61Y. In certain embodiments, the ALK6 ECD polypeptide of the fusion protein comprises one or more substitutions selected from K20Y, H22S and H23G. In certain embodiments, Xaa₁₀₀-Xaa₁₁₃ of the ALK6 ECD polypeptide according to SEQ ID NO: 3 are absent. In certain other embodiments, the ALK6 ECD polypeptide of the fusion protein comprises one or more substitutions selected from K20Y, H22S, H23G and G60K.

In certain embodiments, the ALK6 ECD polypeptide of the fusion protein according to SEQ ID NO: 3 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 1, 4, 11-45, 57, 59, 61, 63, 65, 67, 69, 71, 73, 75, 77, 80, 86, 88, 92, 94, 96, 98, and 100.

In certain embodiments, the soluble ALK6 polypeptide comprises an ECD sequence of ALK6 according to SEQ ID NO: 5 as shown below using standard three-letter amino acid abbreviations:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₂-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₂-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₅₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀;
wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L; and,
Xaa₉₄ is H or Q (SEQ ID NO: 5).

In embodiments, the ALK6 ECD polypeptide of the fusion protein according to SEQ ID NO: 5 comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, or a combination thereof, wherein _ is a deletion at that position. In certain embodiments, the ALK6 ECD polypeptide of the fusion protein according to SEQ ID NO: 5 comprises one or more substitutions selected from L59M, G60K, and L61Y. In certain embodiments, the ALK6 ECD polypeptide of the fusion protein according to SEQ ID NO: 5 comprises one or more substitutions selected from K20Y, H22S and H23G. In certain other embodiments, the ALK6 ECD polypeptide of the fusion protein comprises one or more substitutions selected from K20Y, H22S, H23G and G60K.

In certain embodiments, the soluble ALK6 polypeptide comprises an ECD sequence of ALK6 according to SEQ ID NO: 79 as shown below using standard three-letter amino acid abbreviations:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₂-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₈-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Val₁₀₀-Val₁₀₁-Ile₁₀₂-Gly₁₀₃-Pro₁₀₄-Phe₁₀₅-Phe₁₀₆-Asp₁₀₇-Gly₁₀₈-Ser₁₀₉-Ile₁₁₀-Arg₁₁₁;
wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₅₅ is absent, T or E;
Xaa₈₆ is R or N;
Xaa₈₇ is N or L;
Xaa₈₈ is absent, E or L;
Xaa₉₁ is K or Q;
Xaa₉₂ is D or L; and,
Xaa₉₄ is H or Q (SEQ ID NO: 79).

In embodiments, the ALK6 ECD polypeptide of the fusion protein according to SEQ ID NO: 79 comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position. In certain embodiments, the ALK6 ECD polypeptide of the fusion protein according to SEQ ID NO: 79 comprises one or more substitutions selected from L59M, G60K, and L61Y. In certain embodiments, the ALK6 ECD polypeptide of the fusion protein according to SEQ ID NO: 79 comprises one or more substitutions selected from K20Y, H22S and H23G. In certain other embodiments, the ALK6 ECD polypeptide of the fusion protein comprises one or more substitutions selected from K20Y, H22S, H23G and G60K.

In certain embodiments, the ALK6 ECD polypeptide of the fusion protein according to SEQ ID NO: 79 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 15-45, 57, 59, 61, 63, 65, and 88.

In certain embodiments, the soluble ALK-6 polypeptide comprises an active portion (e.g., ligand binding) of ALK6 according to SEQ ID NO: 81 as shown below using standard three-letter amino acid abbreviations:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₂-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₂-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀-Lys₁₀₁-Asn₁₀₂-Arg₁₀₃-Asp₁₀₄-Phe₁₀₅-Val₁₀₆-Xaa₁₀₇-Xaa₁₀₈-Xaa₁₀₉-Xaa₁₁₀-Xaa₁₁₁-Xaa₁₁₂-Xaa₁₁₃;
wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₇ is absent;
Xaa₁₀₅ is absent;
Xaa₁₀₉ is absent;
Xaa₁₁₀ is absent;
Xaa₁₁₁ is absent;
Xaa₁₁₂ is absent; and,
Xaa₁₁₃ is absent (SEQ ID NO: 81).

In embodiments, the ALK6 ECD polypeptide of the fusion protein according to SEQ ID NO: 81 comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, or a combination thereof, wherein _ is a deletion at that position. In certain embodiments, the ALK6 ECD polypeptide of the fusion protein according to SEQ ID NO: 81 comprises one or more substitutions selected from L59M, G60K, and L61Y. In certain embodiments, the ALK6 ECD polypeptide of the fusion protein according to SEQ ID NO: 81 comprises one or more substitutions selected from K20Y, H22S and H23G. In certain other embodiments, the ALK6 ECD polypeptide of the fusion protein comprises one or more substitutions selected from K20Y, H22S, H23G and G60K.

In certain embodiments, the ALK6 ECD polypeptide of the fusion protein according to SEQ ID NO: 81 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 13, 86, 92, 94, 96, 98, and 100.

In embodiments, the soluble ALK-6 polypeptide comprises an active portion (e.g., ligand binding) of ALK6 according to SEQ ID NO: 82 as shown below using standard three-letter amino acid abbreviations:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₂-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₅₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀-Lys₁₀₁-Asn₁₀₂-Arg₁₀₃-Asp₁₀₄-Phe₁₀₅-Val₁₀₆-Xaa₁₀₇-Gly₁₀₈-Xaa₁₀₉-Ile₁₁₀-Xaa₁₁₁-Xaa₁₁₂-Arg₁₁₃;
wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₇ is D or A;
Xaa₁₀₉ is P or S;
Xaa₁₁₁ is absent; and,
Xaa₁₁₂ is absent (SEQ ID NO: 82).

In embodiments, the ALK6 ECD polypeptide of the fusion protein according to SEQ ID NO: 82 comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position. In certain embodiments, the ALK6 ECD polypeptide of the fusion protein according to SEQ ID NO: 82 comprises one or more substitutions selected from L59M, G60K, and L61Y. In certain embodiments, the ALK6 ECD polypeptide of the fusion protein according to SEQ ID NO: 82 comprises one or more substitutions selected from K20Y, H22S and H23G. In certain other embodiments, the ALK6 ECD polypeptide of the fusion protein comprises one or more substitutions selected from K20Y, H22S, H23G and G60K.

In certain embodiments, the ALK6 ECD polypeptide of the fusion protein according to SEQ ID NO: 82 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 67, 73, 75, and 77.

In embodiments, the soluble ALK-6 polypeptide comprises an active portion (e.g., ligand binding) of ALK6 according to SEQ ID NO: 83 as shown below using standard three-letter amino acid abbreviations:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₂-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₂-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀-Lys₁₀₁-Asn₁₀₂-Arg₁₀₃-Xaa₁₀₄-Phe₁₀₅-Val₁₀₆-Xaa₁₀₇-Gly₁₀₈-Xaa₁₀₉-Ile₁₁₀-His₁₁₁-His₁₁₂-Arg₁₁₃;
wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₅₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₄ is D or A;
Xaa₁₀₇ is absent or D; and,
Xaa₁₀₉ is P or S. (SEQ ID NO: 83).

In embodiments, the ALK6 ECD polypeptide of the fusion protein according to SEQ ID NO: 83 comprises a substitution selected from K15P, K20Y, K20F, K20V, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position. In certain embodiments, the ALK6 ECD polypeptide of the fusion protein according to SEQ ID NO: 83 comprises one or more substitutions selected from L59M, G60K, and L61Y. In certain embodiments, the ALK6 ECD polypeptide of the fusion protein according to SEQ ID NO: 83 comprises one or more substitutions selected from K20Y, H22S and H23G. In certain other embodiments, the ALK6 ECD polypeptide of the fusion protein comprises one or more substitutions selected from K20Y, H22S, H23G and G60K.

In certain embodiments, the ALK6 ECD polypeptide of the fusion protein according to SEQ ID NO: 83 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 12, 14 and 71.

In embodiments, the soluble ALK-6 polypeptide comprises an active portion (e.g., ligand binding) of ALK6 according to SEQ ID NO: 108 as shown below using standard three-letter amino acid abbreviations:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa′₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₂-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₈-Thr₉₆-Leu₉₇-Pro₉₈-Pro₉₉-Leu₁₀₀-Lys₁₀₁-Asn₁₀₂-Arg₁₀₃-Asp₁₀₄-Phe₁₀₅-Val₁₀₆-Xaa₁₀₇-Xaa₁₀₈-Xaa₁₀₉-Xaa₁₁₀-Xaa₁₁₁-Xaa₁₁₂-Xaa₁₁₃;
   wherein:
   Xaa₁₅ is K or P;
   Xaa₂₀ is V, F, K or Y;
   Xaa₂₂ is G, H or S;
   Xaa′₂₂ is absent or A;
   Xaa₂₃ is S, H or G;
   Xaa₂₇ is E or D;
   Xaa₂₉ is S or A;
   Xaa₃₀ is V or I;
   Xaa₃₃ is I or T;
   Xaa₃₅ is S or I;
   Xaa₃₇ is D or N;
   Xaa₃₉ is Y or H;
   Xaa₄₂ is T or A;
   Xaa₄₃ is M or I;
   Xaa₄₉ is S or Q;
   Xaa₅₁ is L or E;
   Xaa₅₂ is P or T;
   Xaa₅₃ is V or T;
   Xaa₅₄ is V or L;
   Xaa₅₅ is T or A;
   Xaa₅₉ is L or M;
   Xaa₆₀ is G or K;
   Xaa₆₁ is L or Y;
   Xaa₆₉ is R or K;
   Xaa₇₁ is T or S;
   Xaa₇₃ is I or K;
   Xaa₇₄ is P or A;
   Xaa₇₅ is H or Q;
   Xaa₇₆ is Q or L;
   Xaa₇₉ is S or T;
   Xaa₈₄ is T or R;
   Xaa₈₅ is absent, T or E;
   Xaa₈₆ is R or N;
   Xaa₈₇ is N or L;
   Xaa₈₈ is absent, E or L;
   Xaa₉₁ is K or Q;
   Xaa₉₂ is D or L;
   Xaa₉₄ is H or Q;
   Xaa₁₀₇ is absent;
   Xaa₁₀₅ is absent;
   Xaa₁₀₉ is absent;
   Xaa₁₁₀ is absent;
   Xaa₁₁₁ is absent;
   Xaa₁₁₂ is absent; and,
   Xaa₁₁₃ is absent (SEQ ID NO: 108)

In embodiments, the ALK6 ECD polypeptide of the fusion protein according to SEQ ID NO: 108 comprises a substitution selected from K15P, K20Y, K20V, K20F, H22S, H22G, _22′A, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, or a combination thereof, wherein _ is a deletion at that position. In certain embodiments, the ALK6 ECD polypeptide of the fusion protein according to SEQ ID NO: 108 comprises one or more substitutions selected from L59M, G60K, and L61Y. In certain embodiments, the ALK6 ECD polypeptide of the fusion protein according to SEQ ID NO: 108 comprises one or more substitutions selected from K20Y, H22S and H23G. In certain other embodiments, the ALK6 ECD polypeptide of the fusion protein comprises one or more substitutions selected from K20Y, H22S, H23G and G60K. In certain embodiments, the ALK6 ECD polypeptide of the fusion protein according to SEQ ID NO: 108 comprises one or more substitutions selected from K20Y, H22S, _22′A and H23G. In certain embodiments, the ALK6 ECD polypeptide of the fusion protein according to SEQ ID NO: 108 comprises one or more substitutions selected from K20Y, H22G, _22′A and H23S.

In certain embodiments, the ALK6 ECD polypeptide of the fusion protein according to SEQ ID NO: 108 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 13, 86, 90, 92, 94, 96, 98, 100, 102, 104, and 110.

In embodiments, the soluble ALK-6 polypeptide comprises an active portion (e.g., ligand binding) of ALK6 according to SEQ ID NO: 109 as shown below using standard three-letter amino acid abbreviations:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa′₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₂-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₂-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Val₁₀₀-Val₁₀₁-Ile₁₀₂-Gly₁₀₃-Pro₁₀₄-Phe₁₀₅-Phe₁₀₆-Asp₁₀₇-Gly₁₀₈-Ser₁₀₉-Ile₁₁₀-Arg₁₁₁;
wherein Xaa₁₅ is K or P;
Xaa₂₀ is V, F, K or Y;
Xaa₂₂ is G, H or S;
Xaa′₂₂ is absent or A;
Xaa₂₃ is S, H or G;
Xaa₂₇ is E or D;
Xaa₂₉ is S or A;
Xaa₃₀ is V or I;
Xaa₃₃ is I or T;
Xaa₅₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L; and,
Xaa₉₄ is H or Q. (SEQ ID NO: 109)

In embodiments, the ALK6 ECD polypeptide of the fusion protein according to SEQ ID NO: 109 comprises a substitution selected from K15P, K20Y, K20V, K20F, H22S, H22G, _22′A, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position. In certain embodiments, the ALK6 ECD polypeptide of the fusion protein according to SEQ ID NO: 109 comprises one or more substitutions selected from L59M, G60K, and L61Y. In certain embodiments, the ALK6 ECD polypeptide of the fusion protein according to SEQ ID NO: 109 comprises one or more substitutions selected from K20Y, H22S and H23G. In certain other embodiments, the ALK6 ECD polypeptide of the fusion protein comprises one or more substitutions selected from K20Y, H22S, H23G and G60K. In certain embodiments, the ALK6 ECD polypeptide of the fusion protein according to SEQ ID NO: 109 comprises one or more substitutions selected from K20Y, H22S, _22′A and H23G. In certain embodiments, the ALK6 ECD polypeptide of the fusion protein according to SEQ ID NO: 109 comprises one or more substitutions selected from K20Y, H22G, _22′A and H23S.

In certain embodiments, the ALK6 ECD polypeptide of the fusion protein according to SEQ ID NO: 109 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 15-45, 57, 59, 61, 63, 65, and 88.

In certain embodiments, the soluble ALK-6 polypeptide comprises an active portion (e.g., ligand binding) of SEQ ID NO: 3 (“SEQ ID NO: 1 variants”). In certain embodiments, the soluble ALK-6 polypeptide comprises an active portion (e.g., ligand binding) comprises an ECD sequence of ALK6 selected from exemplified SEQ ID NO: 1, 4, 11-45, 57, 59, 61, 63, 65, 67, 69, 71, 73, 75, 77, 80, 86, 88, 92, 94, 96, 98, and 100.

In certain embodiments, the soluble ALK-6 polypeptide comprises an active portion (e.g., ligand binding) of SEQ ID NO: 108 (“SEQ ID NO: 113 variants”). In certain embodiments, the soluble ALK-6 polypeptide comprises an active portion (e.g., ligand binding) comprises an ECD sequence of ALK6 selected from exemplified SEQ ID NO: 90, 102, 104, and 110.

In certain embodiments, the soluble ALK-6 polypeptide comprises an active portion (e.g., ligand binding) of SEQ ID NO: 83 (“SEQ ID NO:1 variants”). In certain embodiments, the soluble ALK-6 polypeptide comprises an active portion (e.g., ligand binding) comprises an ECD sequence of ALK6 selected from exemplified SEQ ID NO: 12; SEQ ID NO: 14; and SEQ ID NO: 71.

In certain embodiments, the soluble ALK6 polypeptide comprises an ECD sequence of ALK6 according to SEQ ID NO: 12. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 12.

In certain embodiments, the soluble ALK6 polypeptide comprises an ECD sequence of ALK6 according to SEQ ID NO: 14. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 14.

In certain embodiments, the soluble ALK6 polypeptide comprises an ECD sequence of ALK6 according to SEQ ID NO: 71. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 71.

In certain embodiments, the soluble ALK-6 polypeptide comprises an active portion (e.g., ligand binding) of SEQ ID NO: 79 (“SEQ ID NO:11 variants”). In certain embodiments, the soluble ALK-6 polypeptide comprises an active portion (e.g., ligand binding) comprises an ECD sequence of ALK6 selected from exemplified SEQ ID NOs: 15-45, 57, 59, 61, 63, and 65.

In certain embodiments, the soluble ALK6 polypeptide comprises an ECD sequence of ALK6 according to SEQ ID NO: 15. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 15.

In certain embodiments, the soluble ALK6 polypeptide comprises an ECD sequence of ALK6 according to SEQ ID NO: 16. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 16.

In certain embodiments, the soluble ALK6 polypeptide comprises an ECD sequence of ALK6 according to SEQ ID NO: 17. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 17.

In certain embodiments, the soluble ALK6 polypeptide comprises an ECD sequence of ALK6 according to SEQ ID NO: 18. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 18.

In certain embodiments, the soluble ALK6 polypeptide comprises an ECD sequence of ALK6 according to SEQ ID NO: 19. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 19.

In certain embodiments, the soluble ALK6 polypeptide comprises an ECD sequence of ALK6 according to SEQ ID NO: 20. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 20.

In certain embodiments, the soluble ALK6 polypeptide comprises an ECD sequence of ALK6 according to SEQ ID NO: 21. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 21.

In certain embodiments, the soluble ALK6 polypeptide comprises an ECD sequence of ALK6 according to SEQ ID NO: 22. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 22.

In certain embodiments, the soluble ALK6 polypeptide comprises an ECD sequence of ALK6 according to SEQ ID NO: 23. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 23.

In certain embodiments, the soluble ALK6 polypeptide comprises an ECD sequence of ALK6 according to SEQ ID NO: 24. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 24.

In certain embodiments, the soluble ALK6 polypeptide comprises an ECD sequence of ALK6 according to SEQ ID NO: 25. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 25.

In certain embodiments, the soluble ALK6 polypeptide comprises an ECD sequence of ALK6 according to SEQ ID NO: 26. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 26.

In certain embodiments, the soluble ALK6 polypeptide comprises an ECD sequence of ALK6 according to SEQ ID NO: 27. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 27.

In certain embodiments, the soluble ALK6 polypeptide comprises an ECD sequence of ALK6 according to SEQ ID NO: 28. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 28.

In certain embodiments, the soluble ALK6 polypeptide comprises an ECD sequence of ALK6 according to SEQ ID NO: 29. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 29.

In certain embodiments, the soluble ALK6 polypeptide comprises an ECD sequence of ALK6 according to SEQ ID NO: 30. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 30.

In certain embodiments, the soluble ALK6 polypeptide comprises an ECD sequence of ALK6 according to SEQ ID NO: 31. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 31.

In certain embodiments, the soluble ALK6 polypeptide comprises an ECD sequence of ALK6 according to SEQ ID NO: 32. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 32.

In certain embodiments, the soluble ALK6 polypeptide comprises an ECD sequence of ALK6 according to SEQ ID NO: 33. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 33.

In certain embodiments, the soluble ALK6 polypeptide comprises an ECD sequence of ALK6 according to SEQ ID NO: 34. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 34.

In certain embodiments, the soluble ALK6 polypeptide comprises an ECD sequence of ALK6 according to SEQ ID NO: 35. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 35.

In certain embodiments, the soluble ALK6 polypeptide comprises an ECD sequence of ALK6 according to SEQ ID NO: 36. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 36.

In certain embodiments, the soluble ALK6 polypeptide comprises an ECD sequence of ALK6 according to SEQ ID NO: 37. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 37.

In certain embodiments, the soluble ALK6 polypeptide comprises an ECD sequence of ALK6 according to SEQ ID NO: 38. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 38.

In certain embodiments, the soluble ALK6 polypeptide comprises an ECD sequence of ALK6 according to SEQ ID NO: 39. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 39.

In certain embodiments, the soluble ALK6 polypeptide comprises an ECD sequence of ALK6 according to SEQ ID NO: 40. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 40.

In certain embodiments, the soluble ALK6 polypeptide comprises an ECD sequence of ALK6 according to SEQ ID NO: 41. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 41.

In certain embodiments, the soluble ALK6 polypeptide comprises an ECD sequence of ALK6 according to SEQ ID NO: 42. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 42.

In certain embodiments, the soluble ALK6 polypeptide comprises an ECD sequence of ALK6 according to SEQ ID NO: 43. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 43.

In certain embodiments, the soluble ALK6 polypeptide comprises an ECD sequence of ALK6 according to SEQ ID NO: 44. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 44.

In certain embodiments, the soluble ALK6 polypeptide comprises an ECD sequence of ALK6 according to SEQ ID NO: 45. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 45.

In certain embodiments, the soluble ALK6 polypeptide comprises an ECD sequence of ALK6 according to SEQ ID NO: 57. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 57.

In certain embodiments, the soluble ALK6 polypeptide comprises an ECD sequence of ALK6 according to SEQ ID NO: 59. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 59.

In certain embodiments, the soluble ALK6 polypeptide comprises an ECD sequence of ALK6 according to SEQ ID NO: 61. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 61.

In certain embodiments, the soluble ALK6 polypeptide comprises an ECD sequence of ALK6 according to SEQ ID NO: 63. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 63.

In certain embodiments, the soluble ALK6 polypeptide comprises an ECD sequence of ALK6 according to SEQ ID NO: 65. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 65.

In certain embodiments, the soluble ALK-6 polypeptide comprises an active portion (e.g., ligand binding) of SEQ ID NO: 81 (“SEQ ID NO:69 variants”. In certain embodiments, the soluble ALK-6 polypeptide comprises an active portion (e.g., ligand binding) comprises an ECD sequence of ALK6 exemplified according to SEQ ID NO: 13.

In certain embodiments, the soluble ALK6 polypeptide comprises an ECD sequence of ALK6 according to SEQ ID NO: 13. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 13.

In certain embodiments, the soluble ALK-6 polypeptide comprises an active portion (e.g., ligand binding) of SEQ ID NO: 82 (“SEQ ID NO:80 variants”). In certain embodiments, the soluble ALK-6 polypeptide comprises an active portion (e.g., ligand binding) comprises an ECD sequence of ALK6 selected from exemplified SEQ ID NO: 67, 73, 75, and 77.

In certain embodiments, the soluble ALK6 polypeptide comprises an ECD sequence of ALK6 according to SEQ ID NO: 67. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 67.

In certain embodiments, the soluble ALK6 polypeptide comprises an ECD sequence of ALK6 according to SEQ ID NO: 73. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 73.

In certain embodiments, the soluble ALK6 polypeptide comprises an ECD sequence of ALK6 according to SEQ ID NO: 75. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 75.

In certain embodiments, the soluble ALK6 polypeptide comprises an ECD sequence of ALK6 according to SEQ ID NO: 77. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 77.

In certain embodiments, the soluble ALK6 polypeptide comprises an ECD sequence of ALK6 according to SEQ ID NO: 86. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 86.

In certain embodiments, the soluble ALK6 polypeptide comprises an ECD sequence of ALK6 according to SEQ ID NO: 88. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 88.

In certain embodiments, the soluble ALK6 polypeptide comprises an ECD sequence of ALK6 according to SEQ ID NO: 90. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 90.

In certain embodiments, the soluble ALK6 polypeptide comprises an ECD sequence of ALK6 according to SEQ ID NO: 92. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 92.

In certain embodiments, the soluble ALK6 polypeptide comprises an ECD sequence of ALK6 according to SEQ ID NO: 94. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 94.

In certain embodiments, the soluble ALK6 polypeptide comprises an ECD sequence of ALK6 according to SEQ ID NO: 96. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 96.

In certain embodiments, the soluble ALK6 polypeptide comprises an ECD sequence of ALK6 according to SEQ ID NO: 98. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 98.

In certain embodiments, the soluble ALK6 polypeptide comprises an ECD sequence of ALK6 according to SEQ ID NO: 100. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 100.

In certain embodiments, the soluble ALK6 polypeptide comprises an ECD sequence of ALK6 according to SEQ ID NO: 102. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 102.

In certain embodiments, the soluble ALK6 polypeptide comprises an ECD sequence of ALK6 according to SEQ ID NO: 104. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 104.

In certain embodiments, the soluble ALK6 polypeptide comprises an ECD sequence of ALK6 according to SEQ ID NO: 110. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 110.

In certain embodiments, the soluble ALK6 polypeptide comprises an ECD sequence of ALK6 according to SEQ ID NO: 113. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 113.

In certain embodiments, the ALK6 ECD polypeptide portion of the soluble ALK6 fusion protein (“BMP antagonist fusion protein”) is selected from the group consisting of: SEQ ID NOs: 1, 3-5, 11-45, 57, 59, 61, 63, 65, 67, 69, 71, 73, 75, 77, 79-83, 86, 88, 90, 92, 94, 96, 98, 100, 102, 104, 108, 109, 110 and 113. For example, the ALK6 ECD polypeptide portion of the BMP antagonist fusion protein may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 1, 3-5, 11-45, 57, 59, 61, 63, 65, 67, 69, 71, 73, 75, 77, 79-83, 86, 88, 90, 92, 94, 96, 98, 100, 102, 104, 108, 109, 110 and 113.

In certain embodiments, ALK6 ECD polypeptide portion of the soluble ALK6 fusion protein (“BMP antagonist fusion protein”) is selected from the group consisting of: SEQ ID NO: 33, 86, 88 and 90. For example, the ALK6 ECD polypeptide portion of the BMP antagonist fusion protein may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 33, 86, 88 and 90.

In embodiments, the ALK6 ECD polypeptide can be adjoined to a heterologous domain sequence which aid formation of homodimer soluble ALK6 polypeptides. In certain embodiments, the heterologous domain can be derived from the constant region of an antibody (i.e., the Fc region). In certain embodiments, the Fc region of the fusion protein is exemplified as:

(SEQ ID NO: 10)
THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQ
QGNVFSCSVMHEALHNHYTQKSLSLSPGK.

In certain embodiments, the Fc region of the fusion protein is exemplified as:

(SEQ ID NO: 106)
THTCPPCPAPEAAGGPSVFLFPPKPKDTLMISRTPEVTCVVVDVSHEDPE
VKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPG__

In certain embodiments, the Fc region of the fusion protein is exemplified as:

(SEQ ID NO: 107)
THTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDK
SRWQQGNVFSCSVMHEALHNHYTQKSLSLSPGK

Other heterologous domain sequences may also be suitable as would be understood by those of ordinary skill in the art.

In embodiments, the ALK6 ECD polypeptide can be adjoined to a heterologous domain sequence as a fusion protein using a linker peptide. In certain embodiments, the linker peptide can be selected from the group consisting of:

(SEQ ID NO: 6)
TGGGGSGGGGSGGGGSGGGGS;
(SEQ ID NO: 7)
TGGG;
and,
(SEQ ID NO: 8)
EAAAKEAAAKEAAAKEAAAK.

Other linker peptides may also be suitable as would be understood by those of ordinary skill in the art. See for example SEQ ID NO: 84 and 85

In embodiments, the fusion protein can include a leader peptide. In certain embodiments, the leader peptide can be MGWSCIILFLVATATGVHS (SEQ ID NO: 9). Other leader peptides may also be suitable as would be understood by those of ordinary skill in the art. One of skill in the art also understands the leader sequence is cleaved as part of processing resulting in a mature processed polypeptide sequence (e.g. SEQ ID NO: 1).

Provided herein are a number of exemplified soluble ALK6 fusion polypeptides (proteins) comprising an ALK6 ECD sequence portion, a linker sequence portion and an Fc sequence portion according to any of the portion sequences (e.g., ALK6 ECD, linker and FC) described above and herein. In certain embodiments, the soluble ALK6 fusion polypeptide comprises an amino acid sequence selected from SEQ ID NO: 46-50, 52-56, 58, 60, 62, 64, 66, 68, 70, 72, 74, 76, 78, 84, 85, 87, 89, 91, 93, 95, 97, 99, 101, 103, 105, 111, and 112.

In certain embodiments, the soluble ALK6 fusion polypeptide comprises an amino acid sequence according to SEQ ID NO: 46. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 46.

In certain embodiments, the soluble ALK6 fusion polypeptide comprises an amino acid sequence according to SEQ ID NO: 47. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 47.

In certain embodiments, the soluble ALK6 fusion polypeptide comprises an amino acid sequence according to SEQ ID NO: 48. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 48.

In certain embodiments, the soluble ALK6 fusion polypeptide comprises an amino acid sequence according to SEQ ID NO: 49. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 49.

In certain embodiments, the soluble ALK6 fusion polypeptide comprises an amino acid sequence according to SEQ ID NO: 50. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 50.

In certain embodiments, the soluble ALK6 fusion polypeptide comprises an amino acid sequence according to SEQ ID NO: 52. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 52.

In certain embodiments, the soluble ALK6 fusion polypeptide comprises an amino acid sequence according to SEQ ID NO: 53. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 53.

In certain embodiments, the soluble ALK6 fusion polypeptide comprises an amino acid sequence according to SEQ ID NO: 54. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 54.

In certain embodiments, the soluble ALK6 fusion polypeptide comprises an amino acid sequence according to SEQ ID NO: 55. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 55.

In certain embodiments, the soluble ALK6 fusion polypeptide comprises an amino acid sequence according to SEQ ID NO: 56. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 56.

In certain embodiments, the soluble ALK6 fusion polypeptide comprises an amino acid sequence according to SEQ ID NO: 60. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 60.

In certain embodiments, the soluble ALK6 fusion polypeptide comprises an amino acid sequence according to SEQ ID NO: 62. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 62.

In certain embodiments, the soluble ALK6 fusion polypeptide comprises an amino acid sequence according to SEQ ID NO: 64. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 64.

In certain embodiments, the soluble ALK6 fusion polypeptide comprises an amino acid sequence according to SEQ ID NO: 66. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 66.

In certain embodiments, the soluble ALK6 fusion polypeptide comprises an amino acid sequence according to SEQ ID NO: 68. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 68.

In certain embodiments, the soluble ALK6 fusion polypeptide comprises an amino acid sequence according to SEQ ID NO: 70. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 70.

In certain embodiments, the soluble ALK6 fusion polypeptide comprises an amino acid sequence according to SEQ ID NO: 72. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 72.

In certain embodiments, the soluble ALK6 fusion polypeptide comprises an amino acid sequence according to SEQ ID NO: 74. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 74.

In certain embodiments, the soluble ALK6 fusion polypeptide comprises an amino acid sequence according to SEQ ID NO: 76. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 76.

In certain embodiments, the soluble ALK6 fusion polypeptide comprises an amino acid sequence according to SEQ ID NO: 78. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 78.

In certain embodiments, the soluble ALK6 fusion polypeptide comprises an amino acid sequence according to SEQ ID NO: 84. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 84.

In certain embodiments, the soluble ALK6 fusion polypeptide comprises an amino acid sequence according to SEQ ID NO: 85. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 85.

In certain embodiments, the soluble ALK6 fusion polypeptide comprises an amino acid sequence according to SEQ ID NO: 87. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 87.

In certain embodiments, the soluble ALK6 fusion polypeptide comprises an amino acid sequence according to SEQ ID NO: 89. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 89.

In certain embodiments, the soluble ALK6 fusion polypeptide comprises an amino acid sequence according to SEQ ID NO: 91. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 91.

In certain embodiments, the soluble ALK6 fusion polypeptide comprises an amino acid sequence according to SEQ ID NO: 93. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 93.

In certain embodiments, the soluble ALK6 fusion polypeptide comprises an amino acid sequence according to SEQ ID NO: 95. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 95.

In certain embodiments, the soluble ALK6 fusion polypeptide comprises an amino acid sequence according to SEQ ID NO: 97. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 97.

In certain embodiments, the soluble ALK6 fusion polypeptide comprises an amino acid sequence according to SEQ ID NO: 99. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 99.

In certain embodiments, the soluble ALK6 fusion polypeptide comprises an amino acid sequence according to SEQ ID NO: 101. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 101.

In certain embodiments, the soluble ALK6 fusion polypeptide comprises an amino acid sequence according to SEQ ID NO: 103. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 103.

In certain embodiments, the soluble ALK6 fusion polypeptide comprises an amino acid sequence according to SEQ ID NO: 105. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 105.

In certain embodiments, the soluble ALK6 fusion polypeptide comprises an amino acid sequence according to SEQ ID NO: 111. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 111.

In certain embodiments, the soluble ALK6 fusion polypeptide comprises an amino acid sequence according to SEQ ID NO: 112. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 112.

In certain embodiments, the soluble ALK6 fusion polypeptides (not including the leader peptide (e.g., SEQ ID NO: 9)) can be selected from the group consisting of: SEQ ID NOs: 46-50, 52-56, 60, 62, 64, 66, 68, 70, 72, 74, 76, 78, 84, 85, 87, 89, 91, 93, 95, 97, 99, 101, 103, 105, 111, and 112. For example, the ALK6 fusion polypeptides may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 46-50, 52-56, 60, 62, 64, 66, 68, 70, 72, 74, 76, 78, 84, 85, 87, 89, 91, 93, 95, 97, 99, 101, 103, 105, 111, and 112.

In certain embodiments, soluble ALK6 fusion polypeptides (not including the leader peptide (e.g., SEQ ID NO: 9)) can be selected from the group consisting of: SEQ ID NOs: 87, 89, 91 and 112. For example, the ALK6 fusion polypeptides may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 87, 89, 91 and 112.

Other fusion proteins may also be derived from this disclosure as would be understood by those of ordinary skill in the art.

### Compositions and Pharmaceutical Formulations

The polypeptides described herein may be combined with one or more pharmaceutically acceptable carriers prior to administration to a host. A pharmaceutically acceptable carrier is a material that is not biologically or otherwise undesirable, e.g., the material may be administered to a subject, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. Suitable pharmaceutical carriers and their formulations are described in, for example, Remington's: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally from about 5 to about 8 or from about 7 to about 7.5. Other carriers include sustained-release preparations such as semipermeable matrices of solid hydrophobic polymers containing polypeptides or fragments thereof. Matrices may be in the form of shaped articles, e.g., films, liposomes or microparticles. It will be apparent to those persons skilled in the art that certain carriers may be more preferable depending upon, for instance, the route of administration and concentration of composition being administered. Carriers are those suitable for administration of polypeptides and/or fragments thereof to humans or other subjects. Pharmaceutical compositions may also include carriers, thickeners, diluents, buffers, preservatives, surface active agents, adjuvants, immunostimulants, in addition to the immunogenic polypeptide. Pharmaceutical compositions may also include one or more active ingredients such as antimicrobial agents, anti-inflammatory agents and anesthetics. The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” is a composition comprising a therapeutically effective amount of polypeptide and/or fusion protein disclosed herein.

Methods for treating one or more disease conditions in a mammalian host comprising administering to the mammal at least one or more effective doses of one or more binding agents (and/or derivative(s) thereof) described herein are also provided, as disclosed in more detail below. In some embodiments, one or more polypeptides and/or fusion proteins and/or compositions comprising the same may be administered in a dosage amount of about 1 to about 50 mg (of polypeptide or protein)/kg, about 1 to about 30 mg/kg, or about 5 to about 30 mg/kg (e.g., about any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, or 40 mg/kg). In certain embodiments, the one or more polypeptides and/or fusion proteins and/or compositions comprising the same may be administered to the mammal (e.g., intradermally, intravenously, orally, rectally) at about 10 mg/kg one or more times. When multiple doses are administered, the doses may comprise about the same or different amount of binding agent in each dose. The doses may also be separated in time from one another by the same or different intervals. For instance, the doses may be separated by about any of 6, 12, 24, 36, 48, 60, 72, 84, or 96 hours, one week, two weeks, three weeks, one month, two months, three months, four months, five months, six months, seven months, eight months, nine months, 10 months, 11 months, 12 months, 1.5 years, 2 years, 3 years, 4 years, 5 years, or any time period before, after, and/or between any of these time periods. In some embodiments, the polypeptides and/or fusion proteins and/or compositions comprising the same may be administered in conjunction with other agents (e.g., anti-infective agents and/or chemotherapeutic agent). Such other agents may be administered about simultaneously with the polypeptides and/or fusion proteins and/or compositions comprising the same, or at a different time and/or frequency. Other embodiments of such methods may also be appropriate as could be readily determined by one of ordinary skill in the art.

### Methods of Treatment

Bone morphogenetic protein (BMP) and transforming growth factor-β (TGF-β)/Activin signaling pathways are known to impact synaptogenesis, axonal and dendritic growth, synaptic transmission, and neuronal survival. Given their involvement in nervous system development and function, there is considerable potential that disruptions in signaling activity and efficacy result in neurological disease. Moreover, it is conceivable that targeted manipulations of these pathways may be effective in abrogating cellular dysfunctions typical of nervous system disorders. The BMP and TGF-β/Activin intercellular signaling pathways represent two evolutionarily related branches, composed of a family of ligands, transmembrane receptors, and signal transducers, that display significant sequence and functional conservation, all together often referred to as TGF-β superfamily, or BMP/TGF-β, signaling. BMP/TGF-β signaling plays critical roles in a multitude of developmental and homeostatic processes in invertebrates and vertebrates alike. B3MP ligands were first identified in demineralized bone with remarkable capabilities as inducers of bone deposition, and TGF-β as a growth factor that could stimulate anchorage-independent growth of fibroblasts in soft agar. In addition to their role in the development and homeostasis of bone, BMPs are well known to provide instructional cues in many aspects of development, as well as in tissue homeostasis later in life. Similarly, TGF-1 signaling regulates cell proliferation, differentiation, and apoptosis during development and also acts as a critical regulator of immune function. While these two related branches share some pathway elements, they often antagonize one another, inducing quite different outcomes in the same cellular or tissue environment.

The role of BMP/TGF-β signaling in nervous system function is not well understood, however the pleiotropic pathways appear to tightly regulate cell communication between both neuronal and non-neuronal components of dynamic neural circuits wherein disruptions in BMP/TGF-β signaling plays a role in a variety of neurological diseases, such as Alzheimer's disease, Parkinson's disease, Angelman Syndrome, multiple sclerosis (MS), and amyotrophic lateral sclerosis (ALS).

TGF-β superfamily ligands are expressed broadly in the brain and spinal cord of the adult central nervous system, as well as in the key musculoskeletal components of the motor circuit, while bone morphogenetic protein (BMP) receptor type 1B (ALK6) are abundantly expressed in astrocytes and to a lesser extent in oligodendrocytes. Extracellular ligands that comprise the large family of dimeric BMP/TGF-β signaling molecules, bind the extracellular domain of a heterotetrameric receptor complex, composed of type I and type II transmembrane serine/threonine kinases, to transduce a signal.

Myelin, the lipid membrane that surrounds axons, is critical for the propagation of nervous impulses and axonal maintenance. The destruction of myelin or lack of myelin formation due to disease or injury causes severe motor and cognitive disability. Regeneration of myelin has been noted in select individuals with multiple sclerosis but is not commonly observed. The reasons for this disparity are not understood nevertheless the observation of remyelination demonstrates that in humans remyelination is possible. Myelin is synthesized as the plasma membrane of the oligodendrocyte in the central nervous system. During development, myelin and oligodendrocytes are generated from oligodendrocyte progenitors through a process modulated by extrinsic growth factors signaling to cell-intrinsic proteins. Among the key extrinsic factors are the bone morphogenetic proteins (BMPs), potent inhibitors of oligodendrocyte differentiation and myelin protein expression, likely serving to regulate myelination temporally and spatially. BMPs also promote astrocyte generation but have an inhibitory role in oligodendrogliogenesis during development,

### Multiple Sclerosis

Multiple Sclerosis (MS) is a chronic neurological disorder characterized by the loss of myelin or demyelination. Myelin is produced by oligodendrocytes in the central nervous system (CNS) wherein a single oligodendrocyte may myelinate up to 80 axon segments to facilitate axonal signaling and provide metabolic support to the axon. Loss of oligodendrocyte and demyelination is associated with axonal damage. Given the importance of myelin to CNS health, a wide body of MS research focuses on understanding the vulnerability of oligodendrocytes and their associated myelin sheaths to identify novel strategies to improve myelin regeneration, otherwise known as remyelination and to slow neurodegeneration.

During demyelinating diseases such as multiple sclerosis and stroke, myelin is destroyed and along with it, the oligodendrocytes that synthesize the myelin, Thus, recovery is limited due to both interruptions in neuronal transmission as well as lack of support for neurons. Although oligodendrocyte progenitor cells remain abundant in the central nervous system, they rarely mature and form new functional myelin in the diseased CNS, In cell culture and in experimental models of demyelinating disease, inhibitory signaling factors decrease myelination and remyelination. One of the most potent of these are the bone morphogenetic proteins (BMPs), a family of proteins that strongly inhibits oligodendrocyte progenitor differentiation and myelination in culture. BMPs are highly expressed in the dorsal CNS during pre-natal development and serve to regulate dorsal ventral patterning. Their expression decreases after birth but is significantly increased in rodent demyelination models such as experimental autoimmune encephalomyelitis, cuprizone ingestion and spinal cord injury. In multiple sclerosis and other demyelinating diseases, BMPs are expressed by immune cells invading the CNS as well as resident CNS cell types, mostly astrocytes and microglia.

The disease course of MS is variable—different stages of the disease are characterized by distinct cellular mechanisms and inflammatory processes that depend on CNS-resident immune cells such as microglia, and the infiltration of peripheral immune cells, such as T- and B-cells, Primary-progressive MS (PPMS) is characterized by a linear worsening of symptoms from disease onset and given that the blood-brain barrier (BBB) is mostly intact, CNS/cerebrospinal fluid-localized mechanisms likely facilitate demyelination. Relapsing-remitting MS (RRMS) is characterized by new lesions that cause distinct spikes in symptomatic disease that are followed by a recovery from disability. Relapsing stages of MS generally involve BBB breakdown and infiltration of T- and B-cells that then propagate inflammation to induce damage, RRMS often evolves into a progressive state with no symptomatic remission, termed secondary-progressive MS (SPMS)

Thus, provided herein are BMP antagonists for use in treating a demyelinating disease. In certain embodiments, the demyelinating disease is multiple sclerosis. As provided herein the BMP antagonists are soluble ALK6 fusion proteins which bind their BMP ligands and sequester the ligands preventing their binding to endogenous transmembrane receptors and thus inhibiting signal transduction. In embodiments provided herein, are methods for treating a demyelinating disease by administering a soluble ALK6 fusion protein to a patient in need thereof. In embodiments, the soluble ALK6 fusion protein is selected from any of the fusion proteins described herein comprising an ALK6 ECD sequence (e.g., SEQ H) NO: 1). In particular, comprising an ALK6 ECD sequence selected from any of SEQ ID NO: 3, 5, 79, 81, 82, 83, 108 and 109. In certain embodiments, the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 1; SEQ ID NO: 4; SEQ ID NO: 11, SEQ ID NO: 69; SEQ ID NO: 80 and SEQ ID NO: 113. In certain embodiments, the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 33; SEQ ID NO: 86; SEQ ID NO: 88, and SEQ ID NO: 90. In certain embodiments, soluble ALK6 fusion polypeptides (not including the leader peptide (e.g., SEQ ID NO: 9)) can be selected from the group consisting of: SEQ ID NOs: 87, 89, 91 and 112. For example, the ALK6 fusion polypeptides may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 87, 89, 91 and 112. In particular, the soluble ALK6 fusion polypeptide for use in treating MS comprises an amino acid sequence that is at least 80%, 85%, 90%, 91%, 92%, 93%, 94%, 95%, 96%, 97%, 98%, or 99% identical to an amino acid sequence according to SEQ ID NO: 87.

In certain embodiments provided herein are methods of treating multiple sclerosis, comprising administering an effective amount of a pharmaceutical composition to a subject in need thereof, wherein the composition comprises a soluble recombinant bone morphogenetic protein receptor type-1B fusion protein comprising an ALK6 ECD polypeptide, a linker sequence and a Fc sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from SEQ ID NO: 1, 4, 11-45, 57, 59, 61, 63, 65, 67, 69, 71, 73, 75, 77, 80, 86, 88, 90, 92, 94, 96, 98, 100, 102 and 113, wherein amino acid position 60 is a glycine (G) residue. In certain embodiments, the ALK6 ECD sequence is selected from SEQ ID NO: 86, 90, 92, 94, 96, 98, 100, and 102. In certain embodiment, the ALK6 ECD sequence comprises at least one point mutation as compared to SEQ ID NO: 1, such as those selected from K20Y, K20F, K20V, H22S, H22G, H23S, and H23G. In certain embodiments, the ALK6 ECD sequence comprises an amino acid insertion between position 22 and position 23 of an alanine (A) residue. In particular, the soluble recombinant bone morphogenetic protein receptor type-1B fusion protein for use in treating MS binds the ligand BMP10.

In certain embodiments provided herein are methods of treating multiple sclerosis, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 87 or SEQ ID NO: 91.

In embodiments provided herein are methods for treating multiple sclerosis with the ALK-6 Fc fusion polypeptides of the disclosure, wherein the ALK6 fusion polypeptide binds BMP10. In embodiments, the methods comprise administering an effective amount of a composition comprising the ALK-6 Fc fusion polypeptide to a human subject in need thereof. In embodiments, the method is effective to alleviate a symptom of or a condition associated with multiple sclerosis. In certain embodiments, the method is effective to increase the time to confirmed disease progression, increase the time to confirmed relapse, decrease the hazard ratio for time to confirmed relapse, reduce brain atrophy, reduce relapse rate, reduce rate of confirmed relapses requiring hospitalization, reduce or inhibit progression of the level of fatigue, improve or inhibit deterioration of the functional status, improve or inhibit deterioration of the general health, reduce MRI-monitored disease activity or reduce cognitive impairment in the human patient.

In embodiments, subject or patient according to the disclosure are patients suffering from relapsing-remitting MS (rrMS) or progressive MS (pMS). rrMS is characterized by unpredictable relapses followed by periods of months to years of relative quiet (remission) with no new signs of disease activity. Deficits that occur during attacks may either resolve or leave problems, the latter in about 40% of attacks and being more common the longer a person has had the disease. This describes the initial course of 80% of individuals with MS. Subjects or patients according to the disclosure are patients suffering from pMS can be split up in primary progressive Multiple Sclerosis (ppMS), secondary progressive Multiple Sclerosis (spMS) and early secondary progressive Multiple Sclerosis (espMS). In certain embodiments, patients are selected from human males or females between 10 and 70 of age, more preferably between 18 and 65 years of age, even more preferably 18 and 55 years of age, and especially 18 and 51 years of age. Since female patients are in the vast majority and often have a higher disease burden, the treatment of female patients is preferred.

Patients suffering from MS can be defined for example as having clinically definite or laboratory-definite MS according to Schumacher or Poser criteria (Schumacher et al., Ann. NY Acad. Sci. 1965; 122:552; Poser et al., Ann. Neurol. 1983; 13:227). In embodiments, the human patient is afflicted with relapsing-remitting multiple sclerosis. In other embodiments, the human patient is afflicted with progressive multiple sclerosis.

“Relapses” involve neurologic problems that occur over a short period, typically days but sometimes as short as hours or even minutes. These attacks most often involve motor, sensory, visual or coordination problems early in the disease. Later, bladder, bowel, sexual and cognitive problems may be shown. Sometimes the attack onset occurs over several weeks. Typical MS relapse involves a period of worsening, with development of neurological deficits, then a plateau, in which the patient is not getting any better but also not getting any worse followed by a recovery period. Recovery usually begins within a few weeks.

The “annualized relapse rate” is the average number of relapses a group of patients in a clinical study have in one year. See e.g. Multiple Sclerosis Coalition. The Use Of Disease-Modifying Therapies In Multiple Sclerosis: Principles and Current Evidence Summary.

In embodiments, the method is effective to increase the time to confirmed disease progression in the human patient. In embodiments, the confirmed disease progression is measured by Kurtzke Expanded Disability Status Scale (EDSS) score. Confirmed disease progression is at least a 1 point increase of the EDSS score. In one embodiment, the patient had confirmed disease progression of at least a 0.5 point increase of the EDSS score. The hazard ratio for no confirmed disability worsening is decreased by 20-60%. The hazard ratio for no confirmed disability worsening is decreased by 30-50%. The hazard ratio for no confirmed disability worsening is decreased by at least 30%. The hazard ratio for no confirmed disability worsening is decreased by at least 40%. The hazard ratio for no confirmed disability worsening is decreased by at least 50%.

In certain embodiments, administration of an effective amount of ALK6 Fc fusion polypeptide to a patient in need thereof is effective to increase time to confirmed relapse in the human patient. In embodiments, the method is effective to decrease the hazard ratio for relapse-free survival by at least 20%; is effective to decrease the hazard ratio for relapse-free survival by at least 30%; is effective to decrease the hazard ratio for relapse-free survival by at least 40%; or effective to decrease the hazard ratio for relapse-free survival by at least 50%.

In certain embodiments, administration of an effective amount of ALK-6 Fc fusion polypeptide to a patient in need thereof is effective to decrease the hazard ratio for time to confirmed relapse in the human patient. In embodiments, the method is effective to decrease the hazard ratio for time to confirmed relapse in the human patient by at least 25%; is effective to decrease the hazard ratio for time to confirmed relapse in the human patient by at least 30%; or is effective to decrease the hazard ratio for time to confirmed relapse in the human patient by at least 40%.

In certain embodiments, the administration of the ALK-6 Fc fusion polypeptide is administered as monotherapy for multiple sclerosis. In other embodiments, the administration of the ALK-6 Fc fusion polypeptide is administered as adjunct therapy with an additional multiple sclerosis treatment. In embodiments, the additional multiple sclerosis treatment is interferon beta 1-a, interferon beta 1-b, glatiramer acetate, mitoxantrone, ocrelizumab, natalizumab, alemtuzumab, dialkyl fumarate, laquinimod, siponimod or fingolimod.

In embodiments, the administration of the ALK-6 Fc fusion polypeptide is effective to reduce the annualized relapse rate in the human patient. In certain embodiments, the relapse rate is reduced by at least 25%. In embodiments, the administration of the ALK-6 Fc fusion polypeptide is effective to improve or inhibit deterioration of the general health in the human patient.

### Acute Disseminated Encephalomyelitis and Acute Hemorrhagic Leukoencephalitis (AHLE)

Acute disseminated encephalomyelitis (ADEM) is a neurological disorder characterized by brief but widespread attacks of inflammation (swelling) in the brain and spinal cord that damages myelin (demyelination). ADEM may be an autoimmune disease (e.g., myelin oligodendrocyte antibody-associated disease (MOGAD)), wherein an infection triggers the immune system to attack the CNS and causing inflammation, however it is also demyelinating disease or inflammatory myelinopathy. Acute hemorrhagic leukoencephalitis (AHLE) (also known as Hurst disease) is a severe form of acute disseminated encephalomyelitis and that is characterized by a brief but intense attacks of inflammation in the brain and spinal cord that damages the myelin (demyelination).

A mechanism that allows for OPC to mature into remyelinating oligodendrocytes that are recruited to demyelinated areas is a useful treatment for ADEM and/or AHLE.

Thus, provided herein are BMP antagonists for use in treating ADEM and/or AHLE. In embodiments provided herein, are methods for treating a ADEM and/or AHLE by administering a soluble ALK6 fusion protein to a patient in need thereof. In embodiments, the soluble ALK6 fusion protein is selected from any of the fusion proteins described herein comprising an ALK6 ECD sequence (e.g., SEQ ID NO: 1). In particular, comprising an ALK6 ECD sequence selected from any of SEQ ID NO: 3, 5, 79, 81, 82 and 83. In certain embodiments, the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 1; SEQ ID NO: 4; SEQ ID NO: 11, SEQ ID NO: 69 and SEQ ID NO: 80.

### Amyotrophic Lateral Sclerosis (ALS)

In certain embodiments, the BMP antagonists of this disclosure can be used to treat amyotrophic lateral sclerosis (ALS), which is a neurodegenerative disorder that mainly involves the motor neurons in the spinal cord and the cerebral cortex. Nevertheless, in the last years, cumulative data have shown that there are other neural structures besides motor neurons that are also affected. Aside from cognitive impairments in up to 50% of patients, other non-motor manifestations have been reported, including extrapyramidal, autonomic, and even sensory abnormalities. Particularly, a small fiber sensory neuropathy has been observed in several studies in ALS patients and in animal models.

ALS is characterized by a progressive loss of motor control accompanied by the death of motor neurons whose cell bodies reside in the brain (upper motor neurons) and/or the spinal cord (lower motor neurons). Neuronal death leads to muscle wasting and atrophy, manifesting as weight loss, muscle weakness, fasciculation, and changes in gait or speech. As the disease progresses, everyday motor tasks become more difficult, and the degeneration of diaphragm innervation ultimately leads to respiratory failure and death. In addition to motor loss, approximately 15% of ALS patients experience frontotemporal dementia (FTD), implicating ALS as a multisystem neurodegenerative disease, known as ALS/FTD.

Cutaneous nerve endings are the terminal portions of axons that raise from the body of the pseudo-unipolar neurons located in the dorsal root ganglia (DRG). These neurons and their sensory fibers are classified in subpopulations according to anatomy, physiology, and neurochemistry considerations and present different molecular marker expressions, distinctive receptor characteristics and functions that could have different vulnerability to the neurodegenerative process in ALS. Based on the size and the molecular characteristics, DRG neurons can be divided into three groups: large, myelinated neurons (Aβ mechanoreceptors fibers and Aaβ proprioceptors, the latter expressing parvalbumin; PV), small myelinated (Aδ fibers), and unmyelinated (C fibers) that may be peptidergic neurons (expressing substance P and calcitonin gene-related peptide CORP) and non-peptidergic (labeled with isolectin B4, IB4) neurons. Parvalbumin expressing neurons in DRG are mostly (≥90%) proprioceptors, while the rest correspond to other mechanoreceptors. Peptidergic and non-peptidergic small sensory neurons share some similarities but have some differences. All fon free endings in epidermis and dermis. CGRP+sensory neurons are small myelinated (Aδ type) and unmyelinated (C type), use glutamate as main neurotransmitter and CGRP and some substance P (SP) as neuromodulators, and are regulated during development by nerve growth factor (NGF). Somatic CGRP sensory fibers act as polymodal nociceptors, responding to high-threshold mechanical, thermal, and chemical stimuli. Unmyelinated CGRP+fibers project predominantly to laminae I and IIo (outer layer) in the ventral horn of the spinal cord, whereas small myelinated CGRP axons innervate laminae I and III/IV. IB4+ axons correspond to unmyelinated C nociceptors. These neurons are regulated by glial cell line derived neurotrophic factor (GDNF), although they are dependent on NGF until early postnatal stage when they switch dependence to GDNF. IB4+ central projections synapse into the Ili (inner layer) Rexed lamina.

In ALS rodent models, elevated level of BMP4 was detected in reactive astrocytes in the lumbar ventral spinal horns of late symptomatic animals, consistent with reports that particular BMP ligands, including BMP4, increase following neuronal injury, in animal models of spinal cord injury (SCI), traumatic brain injury (TBI), or demyelinating disorders, accompanied by a neuroinflammatory response. As a means to block BM P signaling, intrathecal injection of Noggin, an extracellular antagonist of the BMP ligands, was shown to reduce markers of neuroinflammation in the rat SOD1^{H46R} ALS model, and to help maintain myelin integrity in independent neuronal injury models.

Thus, provided herein are BMP antagonists of this disclosure for use in treating a neurodegenerative disease. In certain embodiments, the neurodegenerative disease is ALS. As provided herein the BMP antagonists are soluble ALK6 fusion proteins which bind their BMP ligands and sequester the ligands preventing their binding to endogenous transmembrane receptors and thus inhibiting signal transduction. In embodiments provided herein, are methods for treating a neurodegenerative disease by administering a soluble ALK6 fusion protein to a patient in need thereof. In embodiments, the soluble ALK6 fusion protein is selected from any of the fusion proteins described herein comprising an ALK6 ECD sequence (e.g., SEQ ID NO: 1). In particular, comprising an ALK6 ECD sequence selected from any of SEQ ID NO: 3, 5, 79, 81, 82 and 83. In certain embodiments, the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 1; SEQ ID NO: 4; SEQ ID NO: 11, SEQ ID NO: 69 and SEQ ID NO: 80.

The method of treating according to embodiments of the present invention may slow progression of amyotrophic lateral sclerosis (ALS), reduce intensity of symptoms associated with amyotrophic lateral sclerosis, reduce onset of symptoms associated with amyotrophic lateral sclerosis, reduce weight loss associated with amyotrophic lateral sclerosis, reverse weight loss associated with amyotrophic lateral sclerosis, delay mortality associated with amyotrophic lateral sclerosis, or may result in a combination of the above effects. The symptoms associated with amyotrophic lateral sclerosis may include fine motor function, gross motor function, bulbar function, respiratory function, or a combinations thereof. The symptoms associated with amyotrophic lateral sclerosis may include walking, speech, eating, swallowing, writing, climbing stairs, cutting food, turning in bed, salivation, dressing, maintaining hygiene, breathing, dyspnea, orthopnea, respiratory insufficiency, or a combination thereof

In certain embodiments, the treatment may be carried out for a time period selected from the group consisting of at least about 12 weeks, at least about 24 weeks, at least about 6 months, at least about 1 year, at least about 2 years, at least about 3 years, at least about 4 years, at least about 5 years, at least about 10 years, and until the patient dies. In another embodiment, the treatment may be carried out at least daily for an indefinite amount of time.

In embodiments, the patient may exhibit a greater than 10% improvement in ALS Functional Rating Scale, Revised (ALSFRS-R) score when compared to baseline. For example, the patient may exhibit a greater than 15%, a greater than 20%, a greater than 25%, a greater than 30%, a greater than 35%, a greater than 40%, a greater than 45%, a greater than 50%, a greater than 55%, a greater than 60%, a greater than 65%, a greater than 70%, a greater than 75%, a greater than 80%, a greater than 85%, a greater than 90%, or a greater than 95% improvement in ALS Functional Rating Scale, Revised (ALSFRS-R) score when compared to baseline. In an embodiment, the patient may exhibit a greater than 20% improvement in ALS Functional Rating Scale, Revised (ALSFRS-R) score when compared to baseline. In another embodiment, the patient may exhibit a greater than 30% improvement in ALS Functional Rating Scale, Revised (ALSFRS-R) score when compared to baseline. The improvement may be apparent in a time period selected from the group consisting of less than about 9 months, less than about 8 months, less than about 7 months, less than about 6 months, less than about 5 months, less than about 4 months, less than about 3 months, less than about 2 months, and less than about 1 month. For example, the improvement may be apparent in a time period selected from the group consisting of less than about 9 months, less than about 6 months, less than about 3 months, and less than about 1 month.

In an embodiment, the method may further include monitoring the patient. In certain embodiments, the method may include monitoring the patient for neurofilament light chain (NfL) a general biomarker marker of neuronal damage/neurodegenerative disease. In another embodiment, the method may further include monitoring ALSFRS-R score for the patient. In another embodiment, the method may further include monitoring the patient's fine motor function, gross motor function, bulbar function, respiratory function, or a combinations thereof. In another embodiment, the method may further include monitoring behaviors selected from the group consisting of swallowing, handwriting, speech, ability to walk, ability to climb stairs, ability to dress, ability to maintain hygiene, and combinations thereof. In an embodiment, the method may further include scheduling a doctor visit every 6 months for at least 12 months. In an embodiment, the patient may be predisposed to amyotrophic lateral sclerosis and is not exhibiting symptoms of amyotrophic lateral sclerosis.

### Alzheimer's Disease

Alzheimer's Disease (AD) is a neurodegenerative disease characterized by synaptic injury neuronal loss, and amyloid deposition. AD pathogenesis is complex and is related to the abnormal expression of the amyloid 3 (Δ3), APP, and Tau proteins. Neurogenesis in the mature healthy CNS occurs in the olfactory bulb, subventricular zone, and dentate gyrus (DG) of the hippocampus, and plays a role in memory.

Members of the bone morphogenetic protein (BMP) family of growth factors have been implicated as important regulators of neurogenesis and neuronal cell fate determination during development; however, their role in adult neurogenesis and in AD is less clear. Recent work indicates that adult hippocampal neurogenesis may play a role in normal brain functions, such as memory formation, and accumulating evidence has shown that neurogenesis is impaired in animal models of AD, although the mechanism by which adult neurogenesis is impaired in AD remains ill-defined. However, the observation that BMPs are expressed in the adult brain suggests that they may be also involved in adult neurogenesis. In particular, AP-associated increase of BMP6 expression in the hippocampus of AD patients and AD transgenic mice may play a role in impairing NPC proliferation in the hippocampus

It has been shown that mRNA and protein levels of BMP6, but not BMP2 or BMP7, were significantly increased in the in the dentate gyrus of human AD brains compared with nondemented controls and that levels of BMP6 increased as AD progressed. In a mouse model expressing high levels of human amyloid precursor protein, significantly increased BMP6 levels, accumulation of BMP6 surrounding plaques, and reduced neurogenesis in the dentate gyrus were found.

In addition, BMP4 overexpression may be a potential factor to induce AD. Transgenic mice that overexpress BMP4 exhibit impaired memory via the Morris water maze experiment. Moreover, their hippocampal tissues exhibited high expression levels of AD-related proteins, including APP, Aβ, PSEN-1, Tau, P-Tau (Thr181), and P-Tau (Thr231). In multiple cell lines, the overexpression of BMP4 increased the expression of AD-related proteins, whereas the downregulation of BMP4 demonstrated opposing effects.

Thus, provided herein are BMP antagonists of this disclosure for use in treating a neurodegenerative disease. In certain embodiments, the neurodegenerative disease is Alzheimer's Disease (AD). As provided herein the BMP antagonists are soluble ALK6 fusion proteins which bind their BMP ligands and sequester the ligands preventing their binding to endogenous transmembrane receptors and thus inhibiting signal transduction. In embodiments provided herein, are methods for treating Alzheimer's Disease (AD) by administering a soluble ALK6 fusion protein to a patient in need thereof. In embodiments, the soluble ALK6 fusion protein is selected from any of the fusion proteins described herein comprising an ALK6 ECD sequence (e.g., SEQ ID NO: 1). In particular, comprising an ALK6 ECD sequence selected from any of SEQ ID NO: 3, 5, 79, 81, 82 and 83. In certain embodiments, the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 1; SEQ ID NO: 4; SEQ ID NO: 11, SEQ ID NO: 69 and SEQ ID NO: 80.

Although embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the embodiments, specific terminology will be resorted to for the sake of clarity.

The skilled artisan will understand that the figures, described above, and example, described below, are for illustration purposes only. Neither the figures nor the examples are intended to limit the scope of the disclosed teachings in any way.

SEQUENCE LISTING TABLE

| SEQ | | |
|---|---|---|
| ID | | |
| NO: | Description | Amino acid sequence |
| 1 | Wild type ALK-6 | KKEDGESTAPTPRPKVLRCKCHHHCPEDSVNNICSTDGYCFTMIEEDD |
| | ECD Sequence | SGLPVVTSGCLGLEGSDFQCRDTPIPHQRRSIECCTERNECNKDLHPT |
| | | LPPLKNRDFVDGPIHHR |
| 2 | C terminus ALK 3 | VVIGPFFDGSIR |
| | ECD sequence | |
| 3 | Wild type ALK 6 | Lys₁ - Lys₂ - Glu₃ - Asp₄ - Gly₅ - Glu₆ - Ser₇ - |
| | ECD background | Thr₈ - Ala₉ - Pro₁₀ - Thr₁₁ - Pro₁₂ - Arg₁₃ - Pro₁₄ - |
| | with point | Xaa₁₅ - Val₁₆ - Leu₁₇ - Arg₁₈ - Cys₁₉ - Xaa₂₀ - |
| | mutations with | Cys₂₁ - Xaa₂₂ - Xaa₂₃ - His₂₄ - Cys₂₅ - Pro₂₆ - |
| | ALK6 or ALK3 C | Xaa₂₇ - Asp₂₈ - Xaa₂₉ - Xaa₃₀ - Asn₃₁ - Asn₃₂ - |
| | terminus | Xaa₃₃ - Cys₃₄ - Xaa₃₅ - Thr₃₆ - Xaa₃₇ - Gly₃₈ - |
| | sequence | Xaa₃₉ - Cys₄₀ - Phe₄₁ - Xaa₄₂ - Xaa₄₃ - Ile₄₄ - |
| | | Glu₄₅ - Glu₄₆ - Asp₄₇ - Asp₄₈ - Xaa₄₉ - Gly₅₀ - |
| | | Xaa₅₁ - Xaa₅₂ - Xaa₅₃ - Xaa₅₄ - Xaa₅₅ - Ser₅₆ - |
| | | Gly₅₇ - Cys₅₈ - Xaa₅₉ - Xaa₆₀ - Xaa₆₁ - Glu₆₂ - |
| | | Gly₆₃ - Ser₆₄ - Asp₆₅ - Phe₆₆ - Gln₆₇ - Cys₆₈ - |
| | | Xaa₆₉ - Asp₇₀ - Xaa₇₁ - Pro₇₂ - Xaa₇₃ - Xaa₇₄ - |
| | | Xaa₇₅ - Xaa₇₆ - Arg₇₇ - Arg₇₈ - Xaa₇₉ - Ile₈₀ - Glu₈₁ - |
| | | Cys₈₂ - Cys₈₃ - Xaa₈₄ - Xaa₈₅ - Xaa₈₆ - Xaa₈₇ - |
| | | Xaa₈₈ - Cys₈₉ - Asn₉₀ - Xaa₉₁ - Xaa₉₂ - Leu₉₃ - |
| | | Xaa₉₄ - Pro₉₅ - Thr₉₆ - Leu₉₇ - Pro₉₈ - Pro₉₉ - |
| | | Xaa₁₀₀ - Xaa₁₀₁ - Xaa₁₀₂ - Xaa₁₀₃ - Xaa₁₀₄ - Xaa₁₀₅ - |
| | | Xaa₁₀₆ - Xaa₁₀₇ - Xaa₁₀₈ - Xaa₁₀₉ - Xaa₁₁₀ - Xaa₁₁₁ - |
| | | Xaa₁₁₂ - Xaa₁₁₃ |
| | | wherein: Xaa₁₅ is K or P; |
| | | Xaa₂₀ is V, F, K or Y; |
| | | Xaa₂₂ is G, H or S; |
| | | Xaa₂₃ is S, H or G; |
| | | Xaa₂₇ is E or D; |
| | | Xaa₂₉ is S or A; |
| | | Xaa₃₀ is V or I; |
| | | Xaa₃₃ is I or T; |
| | | Xaa₃₅ is S or I; |
| | | Xaa₃₇ is D or N; |
| | | Xaa₃₉ is Y or H; |
| | | Xaa₄₂ is T or A; |
| | | Xaa₄₃ is M or I; |
| | | Xaa₄₉ is S or Q; |
| | | Xaa₅₁ is L or E; |
| | | Xaa₅₂ is P or T; |
| | | Xaa₅₃ is V or T; |
| | | Xaa₅₄ is V or L; |
| | | Xaa₅₅ is T or A; |
| | | Xaa₅₉ is L or M |
| | | Xaa₆₀ is G or K; |
| | | Xaa₆₁ is L or Y; |
| | | Xaa₆₉ is R or K; |
| | | Xaa₇₁ is T or S; |
| | | Xaa₇₃ is I or K; |
| | | Xaa₇₄ is P or A; |
| | | Xaa₇₅ is H or Q; |
| | | Xaa₇₆ is Q or L; |
| | | Xaa₇₉ is S or T; |
| | | Xaa₈₄ is T or R; |
| | | Xaa₅₅ is absent, T or E; |
| | | Xaa₅₆ is R or N; |
| | | Xaa₅₇ is N or L; |
| | | Xaa₅₅ is absent, E or L; |
| | | Xaa₉₁ is K or Q; |
| | | Xaa₉₂ is D or L; |
| | | Xaa₉₄ is H or Q; |
| | | Xaa₁₀₀ is absent, V or L; |
| | | Xaa₁₀₁ is absent, V or K; |
| | | Xaa₁₀₂ is absent, I or N; |
| | | Xaa₁₀₃ is absent, G or R; |
| | | Xaa₁₀₄ is absent, P, D or A; |
| | | Xaa₁₀₅ is absent or F; |
| | | Xaa₁₀₆ is absent, F or V; |
| | | Xaa₁₀₇ is absent or D; |
| | | Xaa₁₀₈ is absent or G; |
| | | Xaa₁₀₉ is absent, S or P; |
| | | Xaa₁₁₀ is absent or I; |
| | | Xaa₁₁₁ is absent, R or H; |
| | | Xaa₁₁₂ is absent or H; |
| | | and Xaa₁₁₃ is absent or R |
| 4 | Wild type ALK-6 | KKEDGESTAPTPRPKVLRCKCHHHCPEDSVNNICSTDGYCFTMIEE |
| | with C terminus | DDSGLPVVTSGCLGLEGSDFQCRDTPIPHQRRSIECCTERNECNKD |
| | truncated (13 aa | LHPTLPPL_____________ |
| | deleted) | |
| 5 | Wild type ALK-6 | Lys₁ - Lys₂ - Glu₃ - Asp₄ - Gly₅ - Glu₆ - Ser₇ - |
| | ECD with C | Thr₈ - Ala₉ - Pro₁₀ - Thr₁₁ - Pro₁₂ - Arg₁₃ - Pro₁₄ |
| | terminus | - Xaa₁₅ - Val₁₆ - Leu₁₇ - Arg₁₈ - Cys₁₉ - Xaa₂₀ - |
| | truncated | Cys₂₁ - Xaa₂₂ - Xaa₂₃ - His₂₄ - Cys₂₅ - Pro₂₆ - |
| | background with | Xaa₂₇ - Asp₂₈ - Xaa₂₉ - Xaa₃₀ - Asn₃₁ - Asn₃₂ - |
| | point mutations | Xaa₃₃ - Cys₃₄ - Xaa₃₅ - Thr₃₆ - Xaa₃₇ - Gly₃₈ - |
| | | Xaa₃₉ - Cys₄₀ - Phe₄₁ - Xaa₄₂ - Xaa₄₃ - Ile₄₄ - |
| | | Glu₄₅ - Glu₄₆ - Asp₄₇ - Asp₄₈ - Xaa₄₉ - Gly₅₀ - |
| | | Xaa₅₁ - Xaa₅₂ - Xaa₅₃ - Xaa₅₄ - Xaa₅₅ - Ser₅₆ - |
| | | Gly₅₇ - Cys₅₈ - Xaa₅₉ - Xaa₆₀ - Xaa₆₁ - Glu₆₂ - |
| | | Gly₆₃ - Ser₆₄ - Asp₆₅ - Phe₆₆ - Gln₆₇ - Cys₆₈ - |
| | | Xaa₆₉ - Asp₇₀ - Xaa₇₁ - Pro₇₂ - Xaa₇₃ - Xaa₇₄ - |
| | | Xaa₇₅ - Xaa₇₆ - Arg₇₇ - Arg₇₈ - Xaa₇₉ - Ile₈₀ - Glu₈₁ - |
| | | Cys₈₂ - Cys₈₃ - Xaa₈₄ - Xaa₈₅ - Xaa₈₆ - Xaa₈₇ - |
| | | Xaa₈₈ - Cys₈₉ - Asn₉₀ - Xaa₉₁ - Xaa₉₂ - Leu₉₃ - |
| | | Xaa₉₄ - Pro₉₅ - Thr₉₆ - Leu₉₇ - Pro₉₉ - Pro₉₉ - Leu₁₀₀ |
| | | wherein Xaa₁₅ is K or P; |
| | | Xaa₂₀ is V, F, K or Y; |
| | | Xaa₂₂ is G, H or S; |
| | | Xaa₂₃ is S, H or G; |
| | | Xaa₂₇ is E or D; |
| | | Xaa₂₉ is S or A; |
| | | Xaa₃₀ is V or I; |
| | | Xaa₃₃ is I or T; |
| | | Xaa₃₅ is S or I; |
| | | Xaa₃₇ is D or N; |
| | | Xaa₃₉ is Y or H; |
| | | Xaa₄₂ is T or A; |
| | | Xaa₄₃ is M or I; |
| | | Xaa₄₉ is S or Q; |
| | | Xaa₅₁ is L or E; |
| | | Xaa₅₂ is P or T; |
| | | Xaa₅₃ is V or T; |
| | | Xaa₅₄ is V or L; |
| | | Xaa₅₅ is T or A; |
| | | Xaa₅₉ is L or M; |
| | | Xaa₆₀ is G or K; |
| | | Xaa₆₁ is L or Y; |
| | | Xaa₆₉ is R or K; |
| | | Xaa₇₁ is T or S; |
| | | Xaa₇₃ is I or K; |
| | | Xaa₇₄ is P or A; |
| | | Xaa₇₅ is H or Q; |
| | | Xaa₇₆ is Q or L; |
| | | Xaa₇₉ is S or T; |
| | | Xaa₈₄ is T or R; |
| | | Xaa₈₈ is absent, T or E; |
| | | Xaa₈₆ is R or N; |
| | | Xaa₈₇ is N or L; |
| | | Xaa₈₈ is absent, E or L; |
| | | Xaa₉₁ is K or Q; |
| | | Xaa₉₂ is D or L; |
| | | and, Xaa₉₄ is H or Q |
| 6 | Linker sequence | TGGGGGGGGSGGGGSGGGGS |
| 7 | Linker sequence | TGGG |
| 8 | Linker sequence | EAAAKEAAAKEAAAKEAAAK |
| 9 | Leader sequence | MGWSCIILFLVATATGVHS |
| 10 | Fc sequence | THTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDK |
| | | SRWQQGNVFSCSVMHEALHNHYTQKSLSLSPGK |
| 11 | Truncated ALK6 | KKEDGESTAPTPRPKVLRCKCHHHCPEDSVNNICSTDGYCFTMIEEDD |
| | ECD (14 aa | SGLPVVTSGCLGLEGSDFQCRDTPIPHQRRSIECCTERNECNKDLHPT |
| | deleted) with | LPPVVIGPFFDGSIR |
| | ALK3 C terminus | |
| | sequence | |
| 12 | SEQ ID NO: 1 | KKEDGESTAPTPRPKVLRCKCHHHCPEDSVNNICSTDGYCFTMIEEDD |
| | D104A Variant | SGLPVVTSGCLGLEGSDFQCRDTPIPHQRRSIECCTERNECNKDLHPT |
| | | LPPLKNRAFVDGPIHHR |
| 13 | Wild type ALK 6 | KKEDGESTAPTPRPKVLRCKCHHHCPEDSVNNICSTDGYCFTMIEEDD |
| | ECD with D104A | SGLPVVTSGCLGLEGSDFQCRDTPIPHQRRSIECCTERNECNKDLHPT |
| | mutation and C | LPPLKNRAFV_______ |
| | terminus | |
| | truncated | |
| 14 | SEQ ID NO: 1 | KKEDGESTAPTPRPKVLRCKCHHHCPEDSVNNICSTDGYCFTMIEEDD |
| | D104A and E107_ | SGLPVVTSGCLGLEGSDFQCRDTPIPHQRRSIECCTERNECNKDLHPT |
| | deletion Variant | LPPLKNRAF_VGPIHHR |
| 15 | SEQ ID NO: 11 | KKEDGESTAPTPRPKVLRCKCHGHCPEDSVNNICSTNGYCFTIIEEDD |
| | Variant with 6 | SGLPVVTSGCMGYEGSDFQCRDTPKAHLRRSIECCTERNECNKDLHPT |
| | point mutations | LPPVVIGPFFDGSIR |
| | (underlined) | |
| 16 | SEQ ID NO: 11 | KKEDGESTAPTPRPKVLRCKCHHHCPEDSVNNICSTDGYCFTMIEEDD |
| | E85_ Deletion | SGLPVVTSGCLGLEGSDFQCRDTPIPHQRRSIECCT_RNECNKDLHPT |
| | Variant | LPPVVIGPFFDGSIR |
| 17 | SEQ ID NO: 11 | KKEDGESTAPTPRPPVLRCKCHHHCPEDSVNNICSTDGYCFTMIEEDD |
| | K15P Variant | SGLPVVTSGCLGLEGSDFQCRDTPIPHQRRSIECCTERNECNKDLHPT |
| | | LPPVVIGPFFDGSIR |
| 18 | SEQ ID NO: 11 | KKEDGESTAPTPRPKVLRCYCHHHCPEDSVNNICSTDGYCFTMIEEDD |
| | K20Y Variant | SGLPVVTSGCLGLEGSDFQCRDTPIPHQRRSIECCTERNECNKDLHPT |
| | | LPPVVIGPFFDGSIR |
| 19 | SEQ ID NO: 11 | KKEDGESTAPTPRPKVLRCKCSHHCPEDSVNNICSTDGYCFTMIEEDD |
| | H22S Variant | SGLPVVTSGCLGLEGSDFQCRDTPIPHQRRSIECCTERNECNKDLHPT |
| | | LPPVVIGPFFDGSIR |
| 20 | SEQ ID NO: 11 | KKEDGESTAPTPRPKVLRCKCHGHCPEDSVNNICSTDGYCFTMIEEDD |
| | H23G Variant | SGLPVVTSGCLGLEGSDFQCRDTPIPHQRRSIECCTERNECNKDLHPT |
| | | LPPVVIGPFFDGSIR |
| 21 | SEQ ID NO: 11 | KKEDGESTAPTPRPKVLRCYCSGHCPEDSVNNICSTDGYCFTMIEEDD |
| | K20Y, H22S and | SGLPVVTSGCLGLEGSDFQCRDTPIPHQRRSIECCTERNECNKDLHPT |
| | H23G Variant | LPPVVIGPFFDGSIR |
| 22 | SEQ ID NO: 11 | KKEDGESTAPTPRPKVLRCKCHHHCPDDSVNNICSTDGYCFTMIEEDD |
| | E27D Variant | SGLPVVTSGCLGLEGSDFQCRDTPIPHQRRSIECCTERNECNKDLHPT |
| | | LPPVVIGPFFDGSIR |
| 23 | SEQ ID NO: 11 | KKEDGESTAPTPRPKVLRCKCHHHCPEDSINNICSTDGYCFTMIEEDD |
| | V30I Variant | SGLPVVTSGCLGLEGSDFQCRDTPIPHQRRSIECCTERNECNKDLHPT |
| | | LPPVVIGPFFDGSIR |
| 24 | SEQ ID NO: 11 | KKEDGESTAPTPRPKVLRCKCHHHCPEDSVNNTCSTDGYCFTMIEEDD |
| | I33T Variant | SGLPVVTSGCLGLEGSDFQCRDTPIPHQRRSIECCTERNECNKDLHPT |
| | | LPPVVIGPFFDGSIR |
| 25 | SEQ ID NO: 11 | KKEDGESTAPTPRPKVLRCKCHHHCPEDSVNNICITDGYCFTMIEEDD |
| | S35I Variant | SGLPVVTSGCLGLEGSDFQCRDTPIPHQRRSIECCTERNECNKDLHPT |
| | | LPPVVIGPFFDGSIR |
| 26 | SEQ ID NO: 11 | KKEDGESTAPTPRPKVLRCKCHHHCPEDSVNNICSTNGYCFTMIEEDD |
| | D37N Variant | SGLPVVTSGCLGLEGSDFQCRDTPIPHQRRSIECCTERNECNKDLHPT |
| | | LPPVVIGPFFDGSIR |
| 27 | SEQ ID NO: 11 | KKEDGESTAPTPRPKVLRCKCHHHCPEDSVNNICSTDGHCFTMIEEDD |
| | Y39H Variant | SGLPVVTSGCLGLEGSDFQCRDTPIPHQRRSIECCTERNECNKDLHPT |
| | | LPPVVIGPFFDGSIR |
| 28 | SEQ ID NO: 11 | KKEDGESTAPTPRPKVLRCKCHHHCPEDSVNNICSTDGYCFAIIEEDD |
| | T42A and M43I | SGLPVVTSGCLGLEGSDFQCRDTPIPHQRRSIECCTERNECNKDLHPT |
| | Variant | LPPVVIGPFFDGSIR |
| 29 | SEQ ID NO: 11 | KKEDGESTAPTPRPKVLRCKCHHHCPEDSVNNICSTDGYCFAMIEEDD |
| | T42A Variant | SGLPVVTSGCLGLEGSDFQCRDTPIPHQRRSIECCTERNECNKDLHPT |
| | | LPPVVIGPFFDGSIR |
| 30 | SEQ ID NO: 11 | KKEDGESTAPTPRPKVLRCKCHHHCPEDSVNNICSTDGYCFTIIEEDD |
| | M43I Variant | SGLPVVTSGCLGLEGSDFQCRDTPIPHQRRSIECCTERNECNKDLHPT |
| | | LPPVVIGPFFDGSIR |
| 31 | SEQ ID NO: 11 | KKEDGESTAPTPRPKVLRCKCHHHCPEDSVNNICSTDGYCFTMIEEDD |
| | S49Q Variant | QGLPVVTSGCLGLEGSDFQCRDTPIPHQRRSIECCTERNECNKDLHPT |
| | | LPPVVIGPFFDGSIR |
| 32 | SEQ ID NO: 11 | KKEDGESTAPTPRPKVLRCKCHHHCPEDSVNNICSTDGYCFTMIEEDD |
| | S49Q, L51E, | QGETTLASGCLGLEGSDFQCRDTPIPHQRRSIECCTERNECNKDLHPT |
| | P52T, V53T, | LPPVVIGPFFDGSIR |
| | V54L, T55A | |
| | Variant | |
| 33 | SEQ ID NO: 11 | KKEDGESTAPTPRPKVLRCKCHHHCPEDSVNNICSTDGYCFTMIEEDD |
| | L59M, G60K and | SGLPVVTSGCMKYEGSDFQCRDTPIPHQRRSIECCTERNECNKDLHPT |
| | L61Y Variant | LPPVVIGPFFDGSIR |
| 34 | SEQ ID NO: 11 | KKEDGESTAPTPRPKVLRCKCHHHCPEDSVNNICSTDGYCFTMIEEDD |
| | L59M Variant | SGLPVVTSGCMGLEGSDFQCRDTPIPHQRRSIECCTERNECNKDLHPT |
| | | LPPVVIGPFFDGSIR |
| 35 | SEQ ID NO: 11 | KKEDGESTAPTPRPKVLRCKCHHHCPEDSVNNICSTDGYCFTMIEEDD |
| | G60K Variant | SGLPVVTSGCLKLEGSDFQCRDTPIPHQRRSIECCTERNECNKDLHPT |
| | | LPPVVIGPFFDGSIR |
| 36 | SEQ ID NO: 11 | KKEDGESTAPTPRPKVLRCKCHHHCPEDSVNNICSTDGYCFTMIEEDD |
| | L61Y Variant | SGLPVVTSGCLGYEGSDFQCRDTPIPHQRRSIECCTERNECNKDLHPT |
| | | LPPVVIGPFFDGSIR |
| 37 | SEQ ID NO: 11 | KKEDGESTAPTPRPKVLRCKCHHHCPEDSVNNICSTDGYCFTMIEEDD |
| | I73K, P74A, | SGLPVVTSGCLGLEGSDFQCRDTPKAQLRRSIECCTERNECNKDLHPT |
| | H75Q, Q76L | LPPVVIGPFFDGSIR |
| | Variant | |
| 38 | SEQ ID NO: 11 | KKEDGESTAPTPRPKVLRCKCHHHCPEDSVNNICSTDGYCFTMIEEDD |
| | I73K and P74A | SGLPVVTSGCLGLEGSDFQCRDTPKAHQRRSIECCTERNECNKDLHPT |
| | Variant | LPPVVIGPFFDGSIR |
| 39 | SEQ ID NO: 11 | KKEDGESTAPTPRPKVLRCKCHHHCPEDSVNNICSTDGYCFTMIEEDD |
| | H75Q and Q76L | SGLPVVTSGCLGLEGSDFQCRDTPIPQLRRSIECCTERNECNKDLHPT |
| | Variant | LPPVVIGPFFDGSIR |
| 40 | SEQ ID NO: 11 | KKEDGESTAPTPRPKVLRCKCHHHCPEDSVNNICSTDGYCFTMIEEDD |
| | T84R and E88L | SGLPVVTSGCLGLEGSDFQCRDTPIPHQRRSIECCRERNLCNKDLHPT |
| | Variant | LPPVVIGPFFDGSIR |
| 41 | SEQ ID NO: 11 | KKEDGESTAPTPRPKVLRCKCHHHCPEDSVNNICSTDGYCFTMIEEDD |
| | G60K, I73K, | SGLPVVTSGCLKLEGSDFQCRDTPKAQLRRSIECCTERNECNKDLHPT |
| | P74A, H75Q and | LPPVVIGPFFDGSIR |
| | Q76L Variant | |
| 42 | SEQ ID NO: 11 | KKEDGESTAPTPRPKVLRCKCHHHCPEDSVNNICSTDGYCFTMIEEDD |
| | T84R, E85T, | SGLPVVTSGCLGLEGSDFQCRDTPIPHQRRSIECCRTNL_CNQYLQPT |
| | R86N, N87L, | LPPVVIGPFFDGSIR |
| | E88_, K91Q, D92Y | |
| | and H94Q Variant | |
| 43 | SEQ ID NO: 11 | KKEDGESTAPTPRPKVLRCKCHHHCPEDSVNNICSTDGYCFTMIEEDD |
| | E85_ Variant | SGLPVVTSGCLGLEGSDFQCRDTPIPHQRRSIECCT_RNECNKDLHPT |
| | | LPPVVIGPFFDGSIR |
| 44 | SEQ ID NO: 11 | KKEDGESTAPTPRPKVLRCKCSGHCPDDAVNNTCITNGYCFAIIEEDD |
| | Variant | SGLPVVASGCMKYEGSDFQCKDSPKAQLRRTIECCTERNECNKDLHPT |
| | H22S, H23G, E27D, | LPPVVIGPFFDGSIR |
| | S29A, I33T, S35I, | |
| | D37N, T43A, M43I, | |
| | T55A, L59M, G60K, | |
| | L61Y,R69K, T71S, | |
| | I73K, P74A, H750, | |
| | Q76L, S79T | |
| 45 | SEQ ID NO: 11 | KKEDGESTAPTPRPKVLRCKCHGHCPEDSVNNICSTNGYCFTIIEEDD |
| | Variant H23G, | SGLPVVTSGCMKYEGSDFQCRDTPKAHLRRSIECCTERNECNKDLHPT |
| | D37N, M43I, | LPPVVIGPFFDGSIR |
| | L59M, G60K, | |
| | L61Y, I73K, | |
| | P74A, Q76L | |
| 46 | SEQ ID NO: 46 | KKEDGESTAPTPRPKVLRCKCHHHCPEDSVNNICSTDGYCFTMIEEDD |
| | | SGLPVVTSGCLGLEGSDFQCRDTPIPHQRRSIECCTERNECNKDLHPT |
| | | LPPVVIGPFFDGSIRTGGGGSGGGGSGGGGSGGGGSTHTCPPCPAPEL |
| | | LGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| 47 | SEQ ID NO: 1 | KKEDGESTAPTPRPKVLRCKCHHHCPEDSVNNICSTDGYCFTMIEEDD |
| | D104A Variant | SGLPVVTSGCLGLEGSDFQCRDTPIPHQRRSIECCTERNECNKDLHPT |
| | | LPPLKNRAFVDGPIHHRTGGGGSGGGGSGGGGSGGGGSTHTCPPCPAP |
| | | ELLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPGK |
| 48 | SEQ ID NO: 1 | KKEDGESTAPTPRPKVLRCKCHHHCPEDSVNNICSTDGYCFTMIEEDD |
| | D104A and C | SGLPVVTSGCLGLEGSDFQCRDTPIPHQRRSIECCTERNECNKDLHPT |
| | terminus 7 aa | LPPLKNRAFV_______TGGGGSGGGGSGGGGSGGGGSTHTCPPCPAP |
| | deletion Variant | ELLGGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPGK |
| 49 | Wild type ALK-6 | MGWSCIILFLVATATGVHSKKEDGESTAPTPRPKVLRCKCHHHCPE |
| | ECD Sequence | DSVNNICSTDGYCFTMIEEDDSGLPVVTSGCLGLEGSDFQCRDTPI |
| | (SEQ ID NO: 1) | PHQRRSIECCTERNECNKDLHPTLPPLKNRDFVDGPIHHRTGGGGS |
| | with | GGGGSGGGGSGGGGSTHTCPPCPAPELLGGPSVFLFPPKPKDTLMI |
| | Leader sequence | SRTPEVTCVVVDVSHEDPEVKFNWYVDGVEVHNAKTKPREEQYNST |
| | (SEQ ID NO: 9) | YRVVSVLTVLHQDWLNGKEYKCKVSNKALPAPIEKTISKAKGQPRE |
| | and linker | PQVYTLPPSREEMTKNQVSLTCLVKGFYPSDIAVEWESNGQPENNY |
| | sequence (SEQ ID | KTTPPVLDSDGSFFLYSKLTVDKSRWQQGNVFSCSVMHEALHNHYT |
| | No: 6) are | QKSLSLSPGK |
| | underlined | |
| | followed by Fc- | |
| | domain sequence | |
| | (SEQ ID NO: 10) | |
| 50 | ALK-6 ECD | MGWSCIILFLVATATGVHSKKEDGESTAPTPRPKVLRCKCHHHCPEDS |
| | Sequence with C- | VNNICSTDGYCFTMIEEDDSGLPVVTSGCLGLEGSDFQCRDTPIPHQR |
| | terminus 14 | RSIECCTERNECNKDLHPTLPPLTGGGGSGGGGSGGGGSGGGGSTHTC |
| | amino acids | PPCPAPELLGGPSVFLFPPKPKDTLMISRTPEVTCVVVDVSHEDPEVK |
| | deleted (SEQ ID | FNWYVDGVEVHNAKTKPREEQYNSTYRVVSVLTVLHQDWLNGKEYKCK |
| | NO: 4) with | VSNKALPAPIEKTISKAKGQPREPQVYTLPPSREEMTKNQVSLTCLVK |
| | Leader sequence | GFYPSDIAVEWESNGQPENNYKTTPPVLDSDGSFFLYSKLTVDKSRWQ |
| | (SEQ ID NO: 9) | QGNVFSCSVMHEALHNHYTQKSLSLSPGK |
| | and linker | |
| | sequence (SEQ ID | |
| | No: 6) | |
| | underlined | |
| | followed by Fc- | |
| | domain sequence | |
| | (SEQ ID NO: 10) | |
| 51 | C-terminus 13 | LKNRDFVDGPIHHR |
| | amino acids from | |
| | the ECD of human | |
| | wild type ALK6 | |
| | (SEQ ID NO: 4) | |
| 52 | SEQ ID NO: 52 | KKEDGESTAPTPRPKVLRCKCHGHCPEDSVNNICSTDGYCFTMIEEDD |
| | | SGLPVVTSGCLGLEGSDFQCRDTPIPHQRRSIECCTERNECNKDLHPT |
| | | LPPVVIGPFFDGSIRTGGGGSGGGGSGGGGSGGGGSTHTCPPCPAPEL |
| | | LGGPSVFLFPPKPKDTLMISRTPEVTCVVVDVSHEDPEVKENWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| 53 | SEQ ID NO: 53 | KKEDGESTAPTPRPKVLRCKCHHHCPEDSVNNICSTNGYCFTMIEE |
| | | DDSGLPVVTSGCLGLEGSDFQCRDTPIPHQRRSIECCTERNECNKD |
| | | LHPTLPPVVIGPFFDGSIRTGGGGSGGGGSGGGGSGGGGSTHTCPP |
| | | CPAPELLGGPSVELFPPKPKDTLMISRTPEVTCVVVDVSHEDPEVK |
| | | FNWYVDGVEVHNAKTKPREEQYNSTYRVVSVLTVLHQDWLNGKEYK |
| | | CKVSNKALPAPIEKTISKAKGQPREPQVYTLPPSREEMTKNQVSLT |
| | | CLVKGFYPSDIAVEWESNGQPENNYKTTPPVLDSDGSFFLYSKLTV |
| | | DKSRWQQGNVFSCSVMHEALHNHYTQKSLSLSPGK |
| 54 | SEQ ID NO: 54 | KKEDGESTAPTPRPKVLRCKCHHHCPEDSVNNICSTDGYCFAIIEEDD |
| | | SGLPVVTSGCLGLEGSDFQCRDTPIPHQRRSIECCTERNECNKDLHPT |
| | | LPPVVIGPFFDGSIRTGGGGSGGGGSGGGGSGGGGSTHTCPPCPAPEL |
| | | LGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| 55 | SEQ ID NO: 55 | KKEDGESTAPTPRPKVLRCKCHHHCPEDSVNNICSTDGYCFTMIEEDD |
| | | SGLPVVTSGCMKYEGSDFQCRDTPIPHQRRSIECCTERNECNKDLHPT |
| | | LPPVVIGPFFDGSIRTGGGGSGGGGSGGGGSGGGGSTHTCPPCPAPEL |
| | | LGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| 56 | SEQ ID NO: 56 | KKEDGESTAPTPRPKVLRCKCHHHCPEDSVNNICSTDGYCFTMIEEDD |
| | | SGLPVVTSGCLKLEGSDFQCRDTPKAQLRRSIECCTERNECNKDLHPT |
| | | LPPVVIGPFFDGSIRTGGGGSGGGGSGGGGSGGGGSTHTCPPCPAPEL |
| | | LGGPSVFLFPPKPKDTLMISRTPEVTCVVVDVSHEDPEVKENWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| 57 | SEQ ID NO: 11 | KKEDGESTAPTPRPKVLRCKCSHHCPEDSVNNICSTDGYCFTMIEEDD |
| | Variant H22S | SGLPVVTSGCLGLEGSDFQCRDTPIPHQRRSIECCTERNECNKDLHPT |
| | | LPPVVIGPFFDGSIR |
| 58 | SEQ ID NO: 58 | KKEDGESTAPTPRPKVLRCKCSHHCPEDSVNNICSTDGYCFTMIEEDD |
| | | SGLPVVTSGCLGLEGSDFQCRDTPIPHQRRSIECCTERNECNKDLHPT |
| | | LPPVVIGPFFDGSIRTGGGGSGGGGSGGGGSGGGGSTHTCPPCPAPEL |
| | | LGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| 59 | SEQ ID NO: 11 | KKEDGESTAPTPRPKVLRCKCSGHCPEDSVNNICSTDGYCFTMIEEDD |
| | Variant H22S and | SGLPVVTSGCLGLEGSDFQCRDTPIPHQRRSIECCTERNECNKDLHPT |
| | H23G | LPPVVIGPFFDGSIR |
| 60 | SEQ ID NO: 60 | KKEDGESTAPTPRPKVLRCKCSGHCPEDSVNNICSTDGYCFTMIEEDD |
| | | SGLPVVTSGCLGLEGSDFQCRDTPIPHQRRSIECCTERNECNKDLHPT |
| | | LPPVVIGPFFDGSIRTGGGGSGGGGSGGGGSGGGGSTHTCPPCPAPEL |
| | | LGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| 61 | SEQ ID NO: 11 | KKEDGESTAPTPRPKVLRCYCSGHCPEDSVNNICSTDGYCFTMIEEDD |
| | Variant K20Y, | SGLPVVTSGCLGLEGSDFQCRDTPIPHQRRSIECCTERNECNKDLHPT |
| | H22S and H23G | LPPVVIGPFFDGSIR |
| 62 | SEQ ID NO: 62 | KKEDGESTAPTPRPKVLRCYCSGHCPEDSVNNICSTDGYCFTMIEEDD |
| | | SGLPVVTSGCLGLEGSDFQCRDTPIPHQRRSIECCTERNECNKDLHPT |
| | | LPPVVIGPFFDGSIRTGGGGSGGGGSGGGGSGGGGSTHTCPPCPAPEL |
| | | LGGPSVFLFPPKPKDTLMISRTPEVTCVVVDVSHEDPEVKENWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| 63 | SEQ ID NO: 11 | KKEDGESTAPTPRPKVLRCYCHGHCPEDSVNNICSTDGYCFTMIEEDD |
| | Variant K20Y and | SGLPVVTSGCLGLEGSDFQCRDTPIPHQRRSIECCTERNECNKDLHPT |
| | H23G | LPPVVIGPFFDGSIR |
| 64 | SEQ ID NO: 64 | KKEDGESTAPTPRPKVLRCYCHGHCPEDSVNNICSTDGYCFTMIEEDD |
| | | SGLPVVTSGCLGLEGSDFQCRDTPIPHQRRSIECCTERNECNKDLHPT |
| | | LPPVVIGPFFDGSIRTGGGGSGGGGSGGGGSGGGGSTHTCPPCPAPEL |
| | | LGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| 65 | SEQ ID NO: 11 | KKEDGESTAPTPRPKVLRCYCHHHCPEDSVNNICSTDGYCFTMIEEDD |
| | Variant K20Y | SGLPVVTSGCLGLEGSDFQCRDTPIPHQRRSIECCTERNECNKDLHPT |
| | | LPPVVIGPFFDGSIR |
| 66 | SEQ ID NO: 66 | KKEDGESTAPTPRPKVLRCYCHHHCPEDSVNNICSTDGYCFTMIEEDD |
| | | SGLPVVTSGCLGLEGSDFQCRDTPIPHQRRSIECCTERNECNKDLHPT |
| | | LPPVVIGPFFDGSIRTGGGGSGGGGSGGGGSGGGGSTHTCPPCPAPEL |
| | | LGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPGK |
| 67 | SEQ ID NO: 1 | KKEDGESTAPTPRPKVLRCYCSGHCPEDSVNNICSTDGYCFTMIEEDD |
| | K20Y, H22S, | SGLPVVTSGCLGLEGSDFQCRDTPIPHQRRSIECCTERNECNKDLHPT |
| | H23G, D107A, | LPPLKNRDFVAGPI__R |
| | H111_, and H112_ | |
| | Variant | |
| 68 | SEQ ID NO: 68 | KKEDGESTAPTPRPKVLRCYCSGHCPEDSVNNICSTDGYCFTMIEEDD |
| | | SGLPVVTSGCLGLEGSDFQCRDTPIPHQRRSIECCTERNECNKDLHPT |
| | | LPPLKNRDFVAGPI__RTGGGGSGGGGSGGGGSGGGGSTHTCPPCPAP |
| | | ELLGGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVES |
| | | CSVMHEALHNHYTQKSLSLSPGK |
| 69 | SEQ ID NO: 1 | KKEDGESTAPTPRPKVLRCKCHHHCPEDSVNNICSTDGYCFTMIEEDD |
| | Variant C- | SGLPVVTSGCLGLEGSDFQCRDTPIPHQRRSIECCTERNECNKDLHPT |
| | terminus seven | LPPLKNRDFV_______ |
| | amino acid | |
| | deletion | |
| | Wild type ALK-6 | |
| | with C terminus | |
| | truncated (7 aa | |
| | deleted) | |
| 70 | SEQ ID NO: 70 | KKEDGESTAPTPRPKVLRCKCHHHCPEDSVNNICSTDGYCFTMIEEDD |
| | | SGLPVVTSGCLGLEGSDFQCRDTPIPHQRRSIECCTERNECNKDLHPT |
| | | LPPLKNRDFV_______TGGGGSGGGGSGGGGSGGGGSTHTCPPCPAP |
| | | ELLGGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVES |
| | | CSVMHEALHNHYTQKSLSLSPGK |
| 71 | SEQ ID NO: 1 | KKEDGESTAPTPRPKVLRCKCHHHCPEDSVNNICSTDGYCFTMIEEDD |
| | D107A Variant | SGLPVVTSGCLGLEGSDFQCRDTPIPHQRRSIECCTERNECNKDLHPT |
| | | LPPLKNRDFVAGPIHHR |
| 72 | SEQ ID NO: 72 | KKEDGESTAPTPRPKVLRCKCHHHCPEDSVNNICSTDGYCFTMIEEDD |
| | | SGLPVVTSGCLGLEGSDFQCRDTPIPHQRRSIECCTERNECNKDLHPT |
| | | LPPLKNRDFVAGPIHHRTGGGGSGGGGSGGGGSGGGGSTHTCPPCPAP |
| | | ELLGGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVES |
| | | CSVMHEALHNHYTQKSLSLSPGK |
| 73 | SEQ ID NO: 1 | KKEDGESTAPTPRPKVLRCKCHHHCPEDSVNNICSTDGYCFTMIEEDD |
| | D107A, H111_ and | SGLPVVTSGCLGLEGSDFQCRDTPIPHQRRSIECCTERNECNKDLHPT |
| | H112_ Variant | LPPLKNRDFVAGPI__R |
| 74 | SEQ ID NO: 74 | KKEDGESTAPTPRPKVLRCKCHHHCPEDSVNNICSTDGYCFTMIEEDD |
| | | SGLPVVTSGCLGLEGSDFQCRDTPIPHQRRSIECCTERNECNKDLHPT |
| | | LPPLKNRDFVAGPI__RTGGGGSGGGGSGGGGSGGGGSTHTCPPCPAP |
| | | ELLGGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPGK |
| 75 | SEQ ID NO: 1 | KKEDGESTAPTPRPKVLRCKCHHHCPEDSVNNICSTDGYCFTMIEEDD |
| | P109S, H111_ and | SGLPVVTSGCLGLEGSDFQCRDTPIPHQRRSIECCTERNECNKDLHPT |
| | H112_ Variant | LPPLKNRDFVDGSI__R |
| 76 | SEQ ID NO: 76 | KKEDGESTAPTPRPKVLRCKCHHHCPEDSVNNICSTDGYCFTMIEEDD |
| | | SGLPVVTSGCLGLEGSDFQCRDTPIPHQRRSIECCTERNECNKDLHPT |
| | | LPPLKNRDFVDGSI__RTGGGGSGGGGSGGGGSGGGGSTHTCPPCPAP |
| | | ELLGGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPGK |
| 77 | SEQ ID NO: 1 | KKEDGESTAPTPRPKVLRCKCHHHCPEDSVNNICSTDGYCFTMIEEDD |
| | P109S, H111_ and | SGLPVVTSGCLGLEGSDFQCRDTPIPHQRRSIECCTERNECNKDLHPT |
| | H112_ Variant | LPPLKNRDFFDGPI__R |
| 78 | SEQ ID NO: 78 | KKEDGESTAPTPRPKVLRCKCHHHCPEDSVNNICSTDGYCFTMIEEDD |
| | | SGLPVVTSGCLGLEGSDFQCRDTPIPHQRRSIECCTERNECNKDLHPT |
| | | LPPLKNRDFFDGPI__RTGGGGSGGGGSGGGGSGGGGSTHTCPPCPAP |
| | | ELLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVES |
| | | CSVMHEALHNHYTQKSLSLSPGK |
| 79 | Truncated ALK6 | Lys₁ - Lys₂ - Glu₃ - Asp₄ - Gly₅ - Glu₆ - |
| | extracellular | Ser₇ - Thr₈ - Ala₉ - Pro₁₀ - Thr₁₁ - Pro₁₂ - |
| | domain (ECD) | Arg₁₃ - Pro₁₄ - Xaa₁₅ - Val₁₆ - Leu₁₇ - Arg₁₈ - |
| | combined with an | Cys₁₉ - Xaa₂₀ - Cys₂₁ - Xaa₂₂ - Xaa₂₃ - His₂₄ - |
| | ALK3 C terminus | Cys₂₅ - Pro₂₆ - Xaa₂₇ - Asp₂₈ - Xaa₂₉ - Xaa₃₀ - |
| | sequence (from | Asn₃₁ - Asn₃₂ - Xaa₃₃ - Cys₃₄ - Xaa₃₅ - Thr₃₆ - |
| | SEQ ID NO: 11) | Xaa₃₇ - Gly₃₈ - Xaa₃₉ - Cys₄₀ - Phe₄₁ - Xaa₄₂ - |
| | | Xaa₄₃ - Ile₄₄ - Glu₄₅ - Glu₄₆ - Asp₄₇ - Asp₄₈ - |
| | | Xaa₄₉ - Gly₅₀ - Xaa₅₁ - Xaa₅₂ - Xaa₅₃ - Xaa₅₄ - |
| | | Xaa₅₅ - Ser₅₆ - Gly₅₇ - Cys₅₈ - Xaa₅₉ - Xaa₆₀ - |
| | | Xaa₆₁ - Glu₆₂ - Gly₆₃ - Ser₆₄ - Asp₆₅ - Phe₆₆ - |
| | | Gln₆₇ - Cys₆₈ - Xaa₆₉ - Asp₇₀ - Xaa₇₁ - Pro₇₂ - |
| | | Xaa₇₃ - Xaa₇₄ - Xaa₇₅ - Xaa₇₆ - Arg₇₇ - Arg₇₈ - |
| | | Xaa₇₉ - Ile₈₀ - Glu₈₁ - Cys₈₂ - Cys₈₃ - Xaa₈₄ - |
| | | Xaa₈₅ - Xaa₈₆ - Xaa₈₇ - Xaa₈₈ - Cys₈₉ - |
| | | Asn₉₀ - Xaa₉₁ - Xaa₉₂ - Leu₉₃ - Xaa₉₄ - Pro₉₅ - |
| | | Thr₉₆ - Leu₉₇ - Pro₉₈ - Pro₉₉ - Val₁₀₀ - Val₁₀₁ - |
| | | Ile₁₀₂ - Gly₁₀₃ - Pro₁₀₄ - Phe₁₀₅ - Phe₁₀₆ - Asp₁₀₇ - |
| | | Gly₁₀₈ - Ser₁₀₉ - Ile₁₁₀ - Arg₁₁₁; |
| | | wherein Xaais is K or P; |
| | | Xaa₂₀ is V, F, K or Y; |
| | | Xaa₂₂ is G, H or S; |
| | | Xaa₂₃ is S, H or G; |
| | | Xaa₂₇ is E or D; |
| | | Xaa₂₉ is S or A; |
| | | Xaa₃₀ is V or I; |
| | | Xaa₃₃ is I or T; |
| | | Xaa₃₅ is S or I; |
| | | Xaa₃₇ is D or N; |
| | | Xaa₃₉ is Y or H; |
| | | Xaa₄₂ is T or A; |
| | | Xaa₄₃ is M or I; |
| | | Xaa₄₉ is S or Q; |
| | | Xaa₅₁ is L or E; |
| | | Xaa₅₂ is P or T; |
| | | Xaa₅₃ is V or T; |
| | | Xaa₅₄ is V or L; |
| | | Xaa₅₅ is T or A; |
| | | Xaa₅₉ is L or M; |
| | | Xaa₆₀ is G or K; |
| | | Xaa₆₁ is L or Y |
| | | Xaa₆₉ is R or K; |
| | | Xaa₇₁ is T or S; |
| | | Xaa₇₃ is I or K; |
| | | Xaa₇₄ is P or A; |
| | | Xaa₇₅ is H or Q; |
| | | Xaa₇₆ is Q or L; |
| | | Xaa₇₉ is S or T; |
| | | Xaa₈₄ is T or R; |
| | | Xaa₈₅ is absent, T or E; |
| | | Xaa₈₆ is R or N; |
| | | Xaa₈₇ is N or L; |
| | | Xaa₈₈ is absent, E or L; |
| | | Xaa₉₁ is K or Q; |
| | | Xaa₉₂ is D or L; |
| | | and, Xaa₉₄ is H or Q |
| 80 | SEQ ID NO: 1 | KKEDGESTAPTPRPKVLRCKCHHHCPEDSVNNICSTDGYCFTMIEEDD |
| | H111_ and H112_ | SGLPVVTSGCLGLEGSDFQCRDTPIPHQRRSIECCTERNECNKDLHPT |
| | Variant | LPPLKNRDFVDGPI__R |
| 81 | Truncated | Lys₁ - Lys₂ - Glu₃ - Asp₄ - Gly₅ - Glu₆ - Ser₇ - Thr₈ - |
| | (7 amino | Ala₉ - Pro₁₀ - Thr₁₁ - Pro₁₂ - Arg₁₃ - Pro₁₄ - Xaa₁₅ - Val₁₆ - |
| | acids) | Leu₁₇ - Arg₁₈ - Cys₁₉ - Xaa₂₀ - Cys₂₁ - Xaa₂₂ - Xaa₂₃ - |
| | ALK6 | His₂₄ - Cys₂₅ - Pro₂₆ - Xaa₂₇ - Asp₂₈ - Xaa₂₉ - Xaa₃₀ - |
| | extracellular | Asn₃₁ - Asn₃₂ - Xaa₃₃ - Cys₃₄ - Xaa₃₅ - Thr₃₆ - Xaa₃₇ - |
| | domain (ECD) | Gly₃₈ - Xaa₃₉ - Cys₄₀ - Phe₄₁ - Xaa₄₂ - Xaa₄₃ - Ile₄₄ - |
| | (From | Glu₄₅ - Glu₄₆ - Asp₄₇ - Asp₄₈ - Xaa₄₉ - Gly₅₀ - Xaa₅₁ - |
| | SEQ ID NO: | Xaa₅₂ - Xaa₅₃ - Xaa₅₄ - Xaa₅₅ - Ser₅₆ - Gly₅₇ - Cys₅₈ - |
| | 1 and 69) | Xaa₅₉ - Xaa₆₀ - Xaa₆₁ - Glu₆₂ - Gly₆₃ - Ser₆₄ - Asp₆₅ - |
| | | Phe₆₆ - Gln₆₇ - Cys₆₈ - Xaa₆₉ - Asp₇₀ - Xaa₇₁ - Pro₇₂ - |
| | | Xaa₇₃ - Xaa₇₄ - Xaa₇₅ - Xaa₇₆ - Arg₇₇ - Arg₇₈ - Xaa₇₉ - Ile₈₀ - |
| | | Glu₈₁ - Cys₈₂ - Cys₈₃ - Xaa₈₄ - Xaa₈₅ - Xaa₈₆ - Xaa₈₇ - |
| | | Xaa₈₈ - Cys₈₉ - Asn₉₀ - Xaa₉₁ - Xaa₉₂ - Leu₉₃ - Xaa₉₄ - |
| | | Pro₉₅ - Thr₉₆ - Leu₉₇ - Pro₉₈ - Pro₉₉ - Leu₁₀₀ - Lys₁₀₁ - |
| | | Asn₁₀₂ - Arg₁₀₃ - Asp₁₀₄ - Phe₁₀₅ - Val₁₀₆ - Xaa₁₀₇ - Xaa₁₀₈ - |
| | | Xaa₁₀₉ - Xaa₁₁₀ - Xaa₁₁₁ - Xaa₁₁₂ - Xaa₁₁₃; |
| | | wherein Xaa₁₅ is K or P; |
| | | Xaa₂₀ is V, F, K or Y; |
| | | Xaa₂₂ is G, H or S; |
| | | Xaa₂₃ is S, H or G; |
| | | Xaa₂₇ is E or D; |
| | | Xaa₂₉ is S or A; |
| | | Xaa₃₀ is V or I; |
| | | Xaa₃₃ is I or T; |
| | | Xaa₃₅ is S or I; |
| | | Xaa₃₇ is D or N; |
| | | Xaa₃₉ is Y or H; |
| | | Xaa₄₂ is T or A; |
| | | Xaa₄₃ is M or I; |
| | | Xaa₄₉ is S or Q; |
| | | Xaa₅₁ is L or E; |
| | | Xaa₅₂ is P or T; |
| | | Xaa₅₃ is V or T; |
| | | Xaa₅₄ is V or L; |
| | | Xaa₅₅ is T or A; |
| | | Xaa₅₉ is L or M; |
| | | Xaa₆₀ is G or K; |
| | | Xaa₆₁ is L or Y; |
| | | Xaa₆₉ is R or K; |
| | | Xaa₇₁ is T or S; |
| | | Xaa₇₃ is I or K; |
| | | Xaa₇₄ is P or A; |
| | | Xaa₇₅ is H or Q; |
| | | Xaa₇₆ is Q or L; |
| | | Xaa₇₉ is S or T; |
| | | Xaa₈₄ is T or R; |
| | | Xaa₈₅ is absent, T or E; |
| | | Xaa₈₆ is R or N; |
| | | Xaa₈₇ is N or L; |
| | | Xaa₈₈ is absent, E or L; |
| | | Xaa₉₁ is K or Q; |
| | | Xaa₉₂ is D or L; |
| | | Xaa₉₄ is H or Q; |
| | | Xaa₁₀₇ is absent; |
| | | Xaa₁₀₈ is absent; |
| | | Xaa₁₀₉ is absent; |
| | | Xaa₁₁₀ is absent; |
| | | Xaa₁₁₁ is absent; |
| | | Xaa₁₁₂ is absent; |
| | | and, Xaa₁₁₃ is absent |
| 82 | Truncated (2 | Lys₁ - Lys₂ - Glu₃ - Asp₄ - Gly₅ - Glu₆ - Ser₇ - Thr₈ - |
| | amino acids) | Ala₉ - Pro₁₀ - Thr₁₁ - Pro₁₂ - Arg₁₃ - Pro₁₄ - Xaa₁₅ - Val₁₆ - |
| | ALK6 | Leu₁₇ - Arg₁₈ - Cys₁₉ - Xaa₂₀ - Cys₂₁ - Xaa₂₂ - Xaa₂₃ - |
| | extracellular | His₂₄ - Cys₂₅ - Pro₂₆ - Xaa₂₇ - Asp₂₈ - Xaa₂₉ - Xaa₃₀ - |
| | domain | Asn₃₁ - Asn₃₂ - Xaa₃₃ - Cys₃₄ - Xaa₃₅ - Thr₃₆ - Xaa₃₇ - |
| | (ECD) (From SEQ | Gly₃₈ - Xaa₃₉ - Cys₄₀ - Phe₄₁ - Xaa₄₂ - Xaa₄₃ - Ile₄₄ - |
| | ID NO: 1 and 80) | Glu₄₅ - Glu₄₆ - Asp₄₇ - Asp₄₈ - Xaa₄₉ - Gly₅₀ - Xaa₅₁ - |
| | | Xaa₅₂ - Xaa₅₃ - Xaa₅₄ - Xaa₅₅ - Ser₅₆ - Gly₅₇ - Cys₅₈ - |
| | | Xaa₅₉ - Xaa₆₀ - Xaa₆₁ - Glu₆₂ - Gly₆₃ - Ser₆₄ - Asp₆₅ - |
| | | Phe₆₆ - Gln₆₇ - Cys₆₈ - Xaa₆₉ - Asp₇o - Xaa₇₁ - Pro₇₂ - |
| | | Xaa₇₃ - Xaa₇₄ - Xaa₇₅ - Xaa₇₆ - Arg₇₇ - Arg₇₈ - Xaa₇₉ - |
| | | Ile₈₀ - Glu₈₁ - Cys₈₂ - Cys₈₃ - Xaa₈₄ - Xaa₈₅ - Xaa₈₆ - |
| | | Xaa₈₇ - Xaa₈₈ - Cys₈₉ - Asn₉₀ - Xaa₉₁ - Xaa₉₂ - Leu₉₃ - |
| | | Xaa₉₄ - Pro₉₅ - Thr₉₆ - Leu₉₇ - Pro₉₈ - Pro₉₉ - Leu₁₀₀ - |
| | | Lys₁₀₁ - Asn₁₀₂ - Arg₁₀₃ - Asp₁₀₄ - Phe₁₀₅ - Val₁₀₆ - |
| | | Xaa₁₀₇ - Gly₁₀₈ - Xaa₁₀₉ - Ile₁₁₀ - Xaa₁₁₁ - Xaa₁₁₂ - Arg₁₁₃; |
| | | wherein Xaa₁₅ is K or P; |
| | | Xaa₂₀ is V, F, K or Y; |
| | | Xaa₂₂ is G, H or S; |
| | | Xaa₂₃ is S, H or G; |
| | | Xaa₂₇ is E or D; |
| | | Xaa₂₉ is S or A; |
| | | Xaa₃₀ is V or I; |
| | | Xaa₃₃ is I or T; |
| | | Xaa₃₅ is S or I; |
| | | Xaa₃₇ is D or N; |
| | | Xaa₃₉ is Y or H; |
| | | Xaa₄₂ is T or A; |
| | | Xaa₄₃ is M or I; |
| | | Xaa₄₉ is S or Q; |
| | | Xaa₅₁ is L or E; |
| | | Xaa₅₂ is P or T; |
| | | Xaa₅₃ is V or T; |
| | | Xaa₅₄ is V or L; |
| | | Xaa₅₅ is T or A; |
| | | Xaa₅₉ is L or M; |
| | | Xaa₆₀ is G or K; |
| | | Xaa₆₁ is L or Y; |
| | | Xaa₆₉ is R or K; |
| | | Xaa₇₁ is T or S; |
| | | Xaa₇₃ is I or K; |
| | | Xaa₇₄ is P or A; |
| | | Xaa₇₅ is H or Q; |
| | | Xaa₇₆ is Q or L; |
| | | Xaa₇₉ is S or T; |
| | | Xaa₈₄ is T or R; |
| | | Xaa₈₅ is absent, T or E; |
| | | Xaa₈₆ is R or N; |
| | | Xaa₈₇ is N or L; |
| | | Xaa₈₈ is absent, E or L; |
| | | Xaa₉₁ is K or Q; |
| | | Xaa₉₂ is D or L; |
| | | Xaa₉₄ is H or Q; |
| | | Xaa₁₀₇ is D or A; |
| | | Xaa₁₀₉ is P or S; |
| | | Xaa₁₁₁ is absent; |
| | | and, Xaa₁₁₂ is absent; |
| 83 | ALK6 | Lys₁ - Lys₂ - Glu₃ - Asp₄ - Gly₅ - Glu₆ - Ser₇ - Thr₈ - Ala₉ - Pro₁₀ - |
| | extracellular | Thr₁₁ - Pro₁₂ - Arg₁₃ - Pro₁₄ - Xaa₁₅ - Val₁₆ - Leu₁₇ - Arg₁₈ - Cys₁₉ - |
| | domain (ECD) with | Xaa₂₀ - Cys₂₁ - Xaa₂₂ - Xaa₂₃ - His₂₄ - Cys₂₅ - Pro₂₆ - Xaa₂₇ - Asp₂₈ - |
| | D104A and D107 | Xaa₂₉ - Xaa₃₀ - Asn₃₁ - Asn₃₂ - Xaa₃₃ - Cys₃₄ - Xaa₃₅ - Thr₃₆ - Xaa₃₇ - |
| | (From SEQ ID NO: | Gly₃₈ - Xaa₃₉ - Cys₄₀ - Phe₄₁ - Xaa₄₂ - Xaa₄₃ - Ile₄₄ - Glu₄₅ - Glu₄₆ - |
| | 1) | Asp₄₇ - Asp₄₈ - Xaa₄₉ - Gly₅₀ - Xaa₅₁ - Xaa₅₂ - Xaa₅₃ - Xaa₅₄ - Xaa₅₅ - |
| | | Ser₅₆ - Gly₅₇ - Cys₅₈ - Xaa₅₉ - Xaa₆₀ - Xaa₆₁ - Glu₆₂ - Gly₆₃ - Ser₆₄ - |
| | | Asp₆₅ - Phe₆₆ - Gln₆₇ - Cys₆₈ - Xaa₆₉ - Asp₇₀ - Xaa₇₁ - Pro₇₂ - Xaa₇₃ - |
| | | Xaa₇₄ - Xaa₇₅ - Xaa₇₆ - Arg₇₇ - Arg₇₈ - Xaa₇₉ - Ile₈₀ - Glu₈₁ - Cys₈₂ - |
| | | Cys₈₃ - Xaa₈₄ - Xaa₈₅ - Xaa₈₆ - Xaa₈₇ - Xaa₈₈ - Cys₈₉ - Asn₉₀ - Xaa₉₁ - |
| | | Xaa₉₂ - Leu₉₃ - Xaa₉₄ - Pro₉₅ - Thr₉₆ - Leu₉₇ - Pro₉₈ - Pro₉₉ - Leu₁₀₀ - |
| | | Lys₁₀₁ - Asn₁₀₂ - Arg₁₀₃ - Xaa₁₀₄ - Phe₁₀₅ - Val₁₀₆ - Xaa₁₀₇ - Gly₁₀₈ - |
| | | Xaa₁₀₉ - Ile₁₁₀ - His₁₁₁ - His₁₁₂ - Arg₁₁₃ |
| | | wherein Xaa₁₅ is K or P; |
| | | Xaa₂₀ is V, F, K or Y; |
| | | Xaa₂₂ is G, H or S; |
| | | Xaa₂₃ is S, H or G; |
| | | Xaa₂₇ is E or D; |
| | | Xaa₂₉ is S or A; |
| | | Xaa₃₀ is V or I; |
| | | Xaa₃₃ is I or T; |
| | | Xaa₃₅ is S or I; |
| | | Xaa₃₇ is D or N; |
| | | Xaa₃₉ is Y or H; |
| | | Xaa₄₂ is T or A; |
| | | Xaa₄₃ is M or I; |
| | | Xaa₄₉ is S or Q; |
| | | Xaa₅₁ is L or E; |
| | | Xaa₅₂ is P or T; |
| | | Xaa₅₃ is V or T; |
| | | Xaa₅₄ is V or L; |
| | | Xaa₅₅ is T or A; |
| | | Xaa₅₉ is L or M; |
| | | Xaa₆₀ is G or K; |
| | | Xaa₆₁ is L or Y; |
| | | Xaa₆₉ is R or K; |
| | | Xaa₇₁ is T or S; |
| | | Xaa₇₃ is I or K; |
| | | Xaa₇₄ is P or A; |
| | | Xaa₇₅ is H or Q; |
| | | Xaa₇₆ is Q or L; |
| | | Xaa₇₉ is S or T; |
| | | Xaa₈₄ is T or R; |
| | | Xaa₈₅ is absent, T or E; |
| | | Xaa₈₆ is R or N; |
| | | Xaa₈₇ is N or L; |
| | | Xaa₈₈ is absent, E or L; |
| | | Xaa₉₁ is K or Q; |
| | | Xaa₉₂ is D or L; |
| | | Xaa₉₄ is H or Q; |
| | | Xaa₁₀₄ is D or A; |
| | | and, Xaa₁₀₇ is absent or D |
| 84 | ECD and Fc same | KKEDGESTAPTPRPKVLRCKCHHHCPEDSVNNICSTDGYCFTMIEEDD |
| | as | SGLPVVTSGCLGLEGSDFQCRDTPIPHQRRSIECCTERNECNKDLHPT |
| | SEQ ID NO: 46; | LPPVVIGPFFDGSIREAAAKEAAAKEAAAKEAAAKTHTCPPCPAPELL |
| | with Alpha helix | GGPSVFLFPPKPKDTLMISRTPEVTCVVVDVSHEDPEVKENWYVDGVE |
| | linker | VHNAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSV |
| | | MHEALHNHYTQKSLSLSPGK |
| 85 | ECD and Fc same | KKEDGESTAPTPRPKVLRCKCHHHCPEDSVNNICSTDGYCFTMIEEDD |
| | as | SGLPVVTSGCLGLEGSDFQCRDTPIPHQRRSIECCTERNECNKDLHPT |
| | SEQ ID NO: 46; | LPPVVIGPFFDGSIRTGGGTHTCPPCPAPELLGGPSVFLFPPKPKDTL |
| | with short | MISRTPEVTCVVVDVSHEDPEVKENWYVDGVEVHNAKTKPREEQYNST |
| | linker | YRVVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKSLSL |
| | | SPGK |
| 86 | SEQ ID NO: 69 | KKEDGESTAPTPRPKVLRCYCSGHCPEDSVNNICSTDGYCFTMIEEDD |
| | and 81 variant | SGLPVVTSGCLGLEGSDFQCRDTPIPHQRRSIECCTERNECNKDLHPT |
| | ECD; K20Y; H22S; | LPPLKNRDFV_______ |
| | H23G; and C- | |
| | terminus seven | |
| | amino acid | |
| | deletion | |
| 87 | SEQ ID NO: 87 | KKEDGESTAPTPRPKVLRCYCSGHCPEDSVNNICSTDGYCFTMIEEDD |
| | | SGLPVVTSGCLGLEGSDFQCRDTPIPHQRRSIECCTERNECNKDLHPT |
| | | LPPLKNRDFV_______TGGGGSGGGGSGGGGSGGGGSTHTCPPCPAP |
| | | EAAGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPG |
| 88 | SEQ ID NO: 11 | KKEDGESTAPTPRPKVLRCYCSGHCPEDSVNNICSTDGYCFTMIEEDD |
| | variant ECD; | SGLPVVTSGCLKLEGSDFQCRDTPIPHQRRSIECCTERNECNKDLHPT |
| | K20Y; H22S; | LPPVVIGPFFDGSIR |
| | H23G; G60K and | |
| | with ALK3 C | |
| | terminus | |
| | sequence | |
| 89 | SEQ ID NO: 89 | KKEDGESTAPTPRPKVLRCYCSGHCPEDSVNNICSTDGYCFTMIEEDD |
| | | SGLPVVTSGCLKLEGSDFQCRDTPIPHQRRSIECCTERNECNKDLHPT |
| | | LPPVVIGPFFDGSIRTGGGGSGGGGSGGGGSGGGGSTHTCPPCPAPEA |
| | | AGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCS |
| | | VMHEALHNHYTQKSLSLSPG |
| 90 | SEQ ID NO: 69 | KKEDGESTAPTPRPKVLRCYCSAGHCPEDSVNNICSTDGYCFTMIEED |
| | and 81 Variant | DSGLPVVTSGCLGLEGSDFQCRDTPIPHQRRSIECCTERNECNKDLHP |
| | ECD; K20Y; H22S; | TLPPLKNRDFV_______ |
| | _22′A (inserted | |
| | between aa22 and | |
| | aa23) H23G; and | |
| | C-terminus seven | |
| | amino acid | |
| | deletion | |
| 91 | SEQ ID NO: 91 | KKEDGESTAPTPRPKVLRCYCSAGHCPEDSVNNICSTDGYCFTMIEED |
| | | DSGLPVVTSGCLGLEGSDFQCRDTPIPHQRRSIECCTERNECNKDLHP |
| | | TLPPLKNRDFV_______TGGGGSGGGGSGGGGSGGGGSTHTCPPCPA |
| | | PEAAGGPSVFLFPPKPKDTLMISRTPEVTCVVVDVSHEDPEVKENWYV |
| | | DGVEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNVE |
| | | SCSVMHEALHNHYTQKSLSLSPG |
| 92 | SEQ ID NO: 69 | KKEDGESTAPTPRPKVLRCYCSGHCPEDSVNNICSTNGYCFTMIEEDD |
| | and 81 Variant; | SGLPVVTSGCLGLEGSDFQCRDTPIPHQRRSIECCTERNECNKDLHPT |
| | ECD K20Y; H22S; | LPPLKNRDFV_______ |
| | H23G; D37N; and | |
| | C-terminus seven | |
| | amino acid | |
| | deletion | |
| 93 | SEQ ID NO: 93 | KKEDGESTAPTPRPKVLRCYCSGHCPEDSVNNICSTNGYCFTMIEEDD |
| | | SGLPVVTSGCLGLEGSDFQCRDTPIPHQRRSIECCTERNECNKDLHPT |
| | | LPPLKNRDFV_______TGGGGSGGGGSGGGGSGGGGSTHTCPPCPAP |
| | | EAAGGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVES |
| | | CSVMHEALHNHYTQKSLSLSPG |
| 94 | SEQ ID NO: 69 | KKEDGESTAPTPRPKVLRCVCSGHCPEDSVNNICSTDGYCFTMIEEDD |
| | and 81 variant | SGLPVVTSGCLGLEGSDFQCRDTPIPHQRRSIECCTERNECNKDLHPT |
| | ECD; K20V; H22S; | LPPLKNRDFV_______ |
| | H23G; and C- | |
| | terminus seven | |
| | amino acid | |
| | deletion | |
| 95 | SEQ ID NO: 95 | KKEDGESTAPTPRPKVLRCVCSGHCPEDSVNNICSTDGYCFTMIEEDD |
| | | SGLPVVTSGCLGLEGSDFQCRDTPIPHQRRSIECCTERNECNKDLHPT |
| | | LPPLKNRDFV_______TGGGGSGGGGSGGGGSGGGGSTHTCPPCPAP |
| | | EAAGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVES |
| | | CSVMHEALHNHYTQKSLSLSPG |
| 96 | SEQ ID NO: 69 | KKEDGESTAPTPRPKVLRCFCSGHCPEDSVNNICSTDGYCFTMIEEDD |
| | and 81 variant | SGLPVVTSGCLGLEGSDFQCRDTPIPHQRRSIECCTERNECNKDLHPT |
| | ECD; K20F; H22S; | LPPLKNRDFV_______ |
| | H23G; and C- | |
| | terminus seven | |
| | amino acid | |
| | deletion | |
| 97 | SEQ ID NO: 97 | KKEDGESTAPTPRPKVLRCFCSGHCPEDSVNNICSTDGYCFTMIEEDD |
| | | SGLPVVTSGCLGLEGSDFQCRDTPIPHQRRSIECCTERNECNKDLHPT |
| | | LPPLKNRDFV_______TGGGGSGGGGSGGGGSGGGGSTHTCPPCPAP |
| | | EAAGGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPG |
| 98 | SEQ ID NO: 69 | KKEDGESTAPTPRPKVLRCYCGSHCPEDSVNNICSTDGYCFTMIEEDD |
| | and 81 variant | SGLPVVTSGCLGLEGSDFQCRDTPIPHQRRSIECCTERNECNKDLHPT |
| | ECD; K20Y; H22G; | LPPLKNRDFV_______ |
| | H23S; and C- | |
| | terminus seven | |
| | amino acid | |
| | deletion | |
| 99 | SEQ ID NO: 99 | KKEDGESTAPTPRPKVLRCYCGSHCPEDSVNNICSTDGYCFTMIEEDD |
| | | SGLPVVTSGCLGLEGSDFQCRDTPIPHQRRSIECCTERNECNKDLHPT |
| | | LPPLKNRDFV_______TGGGGSGGGGSGGGGSGGGGSTHTCPPCPAP |
| | | EAAGGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPG |
| 100 | SEQ ID NO: 69 | KKEDGESTAPTPRPKVLRCFCGSHCPEDSVNNICSTDGYCFTMIEEDD |
| | and 81 variant | SGLPVVTSGCLGLEGSDFQCRDTPIPHQRRSIECCTERNECNKDLHPT |
| | ECD; K20F; H22G; | LPPLKNRDFV_______ |
| | H23S; and C- | |
| | terminus seven | |
| | amino acid | |
| | deletion | |
| 101 | SEQ ID NO: 101 | KKEDGESTAPTPRPKVLRCFCGSHCPEDSVNNICSTDGYCFTMIEEDD |
| | | SGLPVVTSGCLGLEGSDFQCRDTPIPHQRRSIECCTERNECNKDLHPT |
| | | LPPLKNRDFV_______TGGGGSGGGGSGGGGSGGGGSTHTCPPCPAP |
| | | EAAGGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSD |
| | | IAVEWESNGQPENNYKTTPPVLDSDGSFFLYSKLTVDKSRWQQGNVFS |
| | | CSVMHEALHNHYTQKSLSLSPG |
| 102 | SEQ ID NO: 69 | KKEDGESTAPTPRPKVLRCYCGASHCPEDSVNNICSTDGYCFTMIEED |
| | and 81 Variant | DSGLPVVTSGCLGLEGSDFQCRDTPIPHQRRSIECCTERNECNKDLHP |
| | ECD; K20Y; H22G; | TLPPLKNRDFV_______ |
| | _22′A (inserted | |
| | between aa22 and | |
| | aa23) H23S; and | |
| | C-terminus seven | |
| | amino acid | |
| | deletion | |
| 103 | SEQ ID NO: 103 | KKEDGESTAPTPRPKVLRCYCGASHCPEDSVNNICSTDGYCFTMIEED |
| | | DSGLPVVTSGCLGLEGSDFQCRDTPIPHQRRSIECCTERNECNKDLHP |
| | | TLPPLKNRDFV_______TGGGGSGGGGSGGGGSGGGGSTHTCPPCPA |
| | | PEAAGGPSVFLFPPKPKDTLMISRTPEVTCVVVDVSHEDPEVKENWYV |
| | | DGVEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNVF |
| | | SCSVMHEALHNHYTQKSLSLSPG |
| 104 | SEQ ID NO: 69 | KKEDGESTAPTPRPKVLRCYCSAGHCPEDSVNNICSTDGYCFTMIEED |
| | and 81 Variant | DSGLPVVTSGCLKLEGSDFQCRDTPIPHQRRSIECCTERNECNKDLHP |
| | ECD; K20Y; H22S; | TLPPLKNRDFV_______ |
| | _22′A (inserted | |
| | between aa22 and | |
| | aa23) H23G; | |
| | G60K; and C- | |
| | terminus seven | |
| | amino acid | |
| | deletion | |
| 105 | SEQ ID NO: 105 | KKEDGESTAPTPRPKVLRCYCSAGHCPEDSVNNICSTDGYCFTMIEED |
| | | DSGLPVVTSGCLKLEGSDFQCRDTPIPHQRRSIECCTERNECNKDLHP |
| | | TLPPLKNRDFV_______TGGGGSGGGGSGGGGSGGGGSTHTCPPCPA |
| | | PEAAGGPSVFLFPPKPKDTLMISRTPEVTCVVVDVSHEDPEVKENWYV |
| | | DGVEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNVE |
| | | SCSVMHEALHNHYTQKSLSLSPG |
| 106 | Human Fc | THTCPPCPAPEAAGGPSVFLFPPKPKDTLMISRTPEVTCVVVDVSHED |
| | Sequence with | PEVKFNWYVDGVEVHNAKTKPREEQYNSTYRVVSVLTVLHQDWLNGKE |
| | LALA mutation | YKCKVSNKALPAPIEKTISKAKGQPREPQVYTLPPSREEMTKNQVSLT |
| | and (w/o terminal | CLVKGFYPSDIAVEWESNGQPENNYKTTPPVLDSDGSFFLYSKLTVDK |
| | lysine) | SRWQQGNVFSCSVMHEALHNHYTQKSLSLSPG |
| 107 | Human Fc | THTCPPCPAPEAAGGPSVFLFPPKPKDTLMISRTPEVTCVVVDVSHED |
| | Sequence with | PEVKFNWYVDGVEVHNAKTKPREEQYNSTYRVVSVLTVLHQDWLNGKE |
| | LALA mutation | YKCKVSNKALPAPIEKTISKAKGQPREPQVYTLPPSREEMTKNQVSLT |
| | and (w/terminal | CLVKGFYPSDIAVEWESNGQPENNYKTTPPVLDSDGSFFLYSKLTVDK |
| | lysine) | SRWQQGNVFSCSVMHEALHNHYTQKSLSLSPGK |
| 108 | SEQ ID NO: 81 | Lys₁ - Lys₂ - Glu₃ - Asp₄ - Gly₅ - Glu₆ - Ser₇ - Thr₈ - |
| | with addition of | Ala₉ - Pro₁₀ - Thr₁₁ - Pro₁₂ - Arg₁₃ - Pro₁₄ - Xaa₁₅ - Val₁₆ - |
| | aaA between 22 | Leu₁₇ - Arg₁₈ - Cys₁₉ - Xaa₂₀ - Cys₂₁ - Xaa₂₂ - Xaa′₂₂ - |
| | and 23 | Xaa₂₃ - His₂₄ - Cys₂₅ - Pro₂₆ - Xaa₂₇ - Asp₂₈ - Xaa₂₉ - |
| | | Xaa₃₀ - Asn₃₁ - Asn₃₂ - Xaa₃₃ - Cys₃₄ - Xaa₃₅ - Thr₃₆ - |
| | | Xaa₃₇ - Gly₃₈ - Xaa₃₉ - Cys₄₀ - Phe₄₁ - Xaa₄₂ - Xaa₄₃ - |
| | | Ile₄₄ - Glu₄₅ - Glu₄₆ - Asp₄₇ - Asp₄₈ - Xaa₄₉ - Gly₅₀ - |
| | | Xaa₅₁ - Xaa₅₂ - Xaa₅₃ - Xaa₅₄ - Xaa₅₅ - Ser₅₆ - Gly₅₇ - |
| | | Cys₅₈ - Xaa₅₉ - Xaa₆₀ - Xaa₆₁ - Glu₆₂ - Gly₆₃ - Ser₆₄ - |
| | | Asp₆₅ - Phe₆₆ - Gln₆₇ - Cys₆₈ - Xaa₆₉ - Asp₇₀ - Xaa₇₁ - |
| | | Pro₇₂ - Xaa₇₃ - Xaa₇₄ - Xaa₇₅ - Xaa₇₆ - Arg₇₇ - Arg₇₈ - |
| | | Xaa₇₉ - Ile₈₀ - Glu₈₁ - Cys₈₂ - Cys₈₃ - Xaa₈₄ - Xaa₈₅ - Xaa₈₆ - |
| | | Xaa₈₇ - Xaa₈₈ - Cys₈₉ - Asn₉₀ - Xaa₉₁ - Xaa₉₂ - Leu₉₃ - |
| | | Xaa₉₄ - Pro₉₅ - Thr₉₆ - Leu₉₇ - Pro₉₈ - Pro₉₉ - Leu₁₀₀ - |
| | | Lys₁₀₁ - Asn₁₀₂ - Arg₁₀₃ - Asp₁₀₄ - Phe₁₀₅ - Val₁₀₆ - Xaa₁₀₇ - |
| | | Xaa₁₀₈ - Xaa₁₀₉ - Xaa₁₁₀ - Xaa₁₁₁ - Xaa₁₁₂ - Xaa₁₁₃; |
| | | wherein Xaais is K or P; |
| | | Xaa₂₀ is V, F, K or Y; |
| | | Xaa₂₂ is G, H or S; |
| | | Xaa′₂₂ is absent or A; |
| | | Xaa₂₃ is S, H or G; |
| | | Xaa₂₇ is E or D; |
| | | Xaa₂₉ is S or A; |
| | | Xaa₃₀ is V or I; |
| | | Xaa₃₃ is I or T; |
| | | Xaa₃₅ is S or I; |
| | | Xaa₃₇ is D or N; |
| | | Xaa₃₉ is Y or H; |
| | | Xaa₄₂ is T or A; |
| | | Xaa₄₃ is M or I; |
| | | Xaa₄₉ is S or Q; |
| | | Xaa₅₁ is L or E; |
| | | Xaa₅₂ is P or T; |
| | | Xaa₅₃ is V or T; |
| | | Xaa₅₄ is V or L; |
| | | Xaa₅₅ is T or A; |
| | | Xaa₅₉ is L or M; |
| | | Xaa₆₀ is G or K; |
| | | Xaa₆₁ is L or Y; |
| | | Xaa₆₉ is R or K; |
| | | Xaa₇₁ is T or S; |
| | | Xaa₇₃ is I or K; |
| | | Xaa₇₄ is P or A; |
| | | Xaa₇₅ is H or Q; |
| | | Xaa₇₆ is Q or L; |
| | | Xaa₇₉ is S or T; |
| | | Xaa₈₄ is T or R; |
| | | Xaa₈₅ is absent, T or E; |
| | | Xaa₈₆ is R or N; |
| | | Xaa₈₇ is N or L; |
| | | Xaa₈₈ is absent, E or L; |
| | | Xaa₉₁ is K or Q; |
| | | Xaa₉₂ is D or L; |
| | | Xaa₉₄ is H or Q; |
| | | Xaa₁₀₇ is absent; |
| | | Xaa₁₀₈ is absent; |
| | | Xaa₁₀₉ is absent; |
| | | Xaa₁₁₀ is absent; |
| | | Xaa₁₁₁ is absent; |
| | | Xaa₁₁₂ is absent; |
| | | and, Xaa₁₁₃ is absent |
| 109 | SEQ ID NO: 79 | Lys₁ - Lys₂ - Glu₃ - Asp₄ - Gly₅ - Glu₆ - |
| | with addition of | Ser₇ - Thr₈ - Ala₉ - Pro₁₀ - Thr₁₁ - Pro₁₂ - |
| | aaA between 22 | Arg₁₃ - Pro₁₄ - Xaa₁₅ - Val₁₆ - Leu₁₇ - Arg₁₈ - |
| | and 23 | Cys₁₉ - Xaa₂₀ - Cys₂₁ - Xaa₂₂ - Xaa′₂₂ - Xaa₂₃ - |
| | | His₂₄ - Cys₂₅ - Pro₂₆ - Xaa₂₇ - Asp₂₈ - Xaa₂₉ |
| | | - Xaa₃₀ - Asn₃₁ - Asn₃₂ - Xaa₃₃ - Cys₃₄ - Xaa₃₅ |
| | | - Thr₃₆ - Xaa₃₇ - Gly₃₈ - Xaa₃₉ - Cys₄₀ - Phe₄₁ |
| | | - Xaa₄₂ - Xaa₄₃ - Ile₄₄ - Glu₄₅ - Glu₄₆ - Asp₄₇ |
| | | - Asp₄₈ - Xaa₄₉ - Gly₅₀ - Xaa₅₁ - Xaa₅₂ - Xaa₅₃ |
| | | - Xaa₅₄ - Xaa₅₅ - Ser₅₆ - Gly₅₇ - Cys₅₈ - Xaa₅₉ |
| | | - Xaa₆₀ - Xaa₆₁ - Glu₆₂ - Gly₆₃ - Ser₆₄ - Asp₆₅ |
| | | - Phe₆₆ - Gln₆₇ - Cys₆₈ - Xaa₆₉ - Asp₇₀ - Xaa₇₁ |
| | | - Pro₇₂ - Xaa₇₃ - Xaa₇₄ - Xaa₇₅ - Xaa₇₆ - Arg₇₇ |
| | | - Arg₇₈ - Xaa₇₉ - Ile₈₀ - Glu₈₁ - Cys₈₂ - Cys₈₃ |
| | | - Xaa₈₄ - Xaa₈₅ - Xaa₈₆ - Xaa₈₇ - Xaa₈₈ - Cys₈₉ |
| | | - Asn₉₀ - Xaa₉₁ - Xaa₉₂ - Leu₉₃ - Xaa₉₄ - Pro₉₅ |
| | | - Thr₉₆ - Leu₉₇ - Pro₉₈ - Pro₉₉ - Val₁₀₀ - Val₁₀₁ - |
| | | Ile₁₀₂ - Gly₁₀₃ - Pro₁₀₄ - Phe₁₀₅ - Phe₁₀₆ - Asp₁₀₇ - |
| | | Gly₁₀₈ - Ser₁₀₉ - Ile₁₁₀ - Arg₁₁₁; |
| | | wherein Xaais is K or P; |
| | | Xaa₂₀ is V, F, K or Y; |
| | | Xaa₂₂ is G, H or S; |
| | | Xaa′₂₂ is absent or A - Xaa₂₃ is S, H or G; |
| | | Xaa₂₇ is E or D; |
| | | Xaa₂₉ is S or A; |
| | | Xaa₃₀ is V or I; |
| | | Xaa₃₃ is I or T; |
| | | Xaa₃₅ is S or I; |
| | | Xaa₃₇ is D or N; |
| | | Xaa₃₉ is Y or H; |
| | | Xaa₄₂ is T or A; |
| | | Xaa₄₃ is M or I; |
| | | Xaa₄₉ is S or Q; |
| | | Xaa₅₁ is L or E; |
| | | Xaa₅₂ is P or T; |
| | | Xaa₅₃ is V or T; |
| | | Xaa₅₄ is V or L; |
| | | Xaa₅₅ is T or A; |
| | | Xaa₅₉ is L or M; |
| | | Xaa₆₀ is G or K; |
| | | Xaa₆₁ is L or Y |
| | | Xaa₆₉ is R or K; |
| | | Xaa₇₁ is T or S; |
| | | Xaa₇₃ is I or K; |
| | | Xaa₇₄ is P or A; |
| | | Xaa₇₅ is H or Q; |
| | | Xaa₇₆ is Q or L; |
| | | Xaa₇₉ is S or T; |
| | | Xaa₈₄ is T or R; |
| | | Xaa₈₅ is absent, T or E; |
| | | Xaa₈₆ is R or N; |
| | | Xaa₈₇ is N or L; |
| | | Xaa₈₈ is absent, E or L; |
| | | Xaa₉₁ is K or Q; |
| | | Xaa₉₂ is D or L; |
| | | and, Xaa₉₄ is H or Q |
| 110 | SEQ ID NO: 69 | KKEDGESTAPTPRPKVLRCYCSAGHCPEDSVNNICSTDGYC |
| | Variant ECD ; | FTMIEEDDSGLPVVTSGCMKYEGSDFQCRDTPIPHQRRSIE |
| | K20Y; H22S; 22aA | CCTERNECNKDLHPTLPPLKNRDEV |
| | (inserted | |
| | between aa22 and | |
| | aa23) H23G; | |
| | L59M, G60K and | |
| | L61Y and C- | |
| | terminus seven | |
| | amino acid | |
| | deletion | |
| 111 | SEQ ID NO: 111 | KKEDGESTAPTPRPKVLRCYCSAGHCPEDSVNNICSTDGYC |
| | | FTMIEEDDSGLPVVTSGCMKYEGSDFQCRDTPIPHQRRSIE |
| | | CCTERNECNKDLHPTLPPLKNRDFV_______TGGGGSGGG |
| | | GSGGGGSGGGGSTHTCPPCPAPEAAGGPSVELFPPKPKDTL |
| | | MISRTPEVTCVVVDVSHEDPEVKENWYVDGVEVHNAKTKPR |
| | | EEQYNSTYRVVSVLTVLHQDWLNGKEYKCKVSNKALPAPIE |
| | | KTISKAKGQPREPQVYTLPPSREEMTKNQVSLTCLVKGFYP |
| | | SDIAVEWESNGQPENNYKTTPPVLDSDGSFFLYSKLTVDKS |
| | | RWQQGNVFSCSVMHEALHNHYTQKSLSLSPG |
| 112 | SEQ ID NO: 33 + | KKEDGESTAPTPRPKVLRCKCHHHCPEDSVNNICSTD |
| | SEQ ID NO: 6 + | GYCFTMIEEDDSGLPVVTSGCMKYEGSDFQCRDTPIP |
| | SEQ ID NO: 106 | HQRRSIECCTERNECNKDLHPTLPPVVIGPFFDGSIR |
| | | TGGGGSGGGGSGGGGSGGGGSTHTCPPCPAPEAAGGP |
| | | SVFLFPPKPKDTLMISRTPEVTCVVVDVSHEDPEVKF |
| | | NWYVDGVEVHNAKTKPREEQYNSTYRVVSVLTVLHQD |
| | | WLNGKEYKCKVSNKALPAPIEKTISKAKGQPREPQVY |
| | | TLPPSREEMTKNQVSLTCLVKGFYPSDIAVEWESNGQ |
| | | PENNYKTTPPVLDSDGSFFLYSKLTVDKSRWQQGNVE |
| | | SCSVMHEALHNHYTQKSLSLSPG |
| 113 | SEQ ID NO: 69 | KKEDGESTAPTPRPKVLRCKCHAHHCPEDSVNNICSTDGYCFTMIEED |
| | background with | DSGLPVVTSGCLGLEGSDFQCRDTPIPHQRRSIECCTERNECNKDLHP |
| | aaA inserted | TLPPLKNRDFV_______ |
| | between aa22 and | |
| | aa23 | |
| 114 | ALK6-Fc fusion | KKEDGESTAPTPRPKVLRCKCHAHHCPEDSVNNICSTDGYCFTMIEED |
| | polypeptide | DSGLPVVTSGCLGLEGSDFQCRDTPIPHQRRSIECCTERNECNKDLHP |
| | _22′ A | TLPPLKNRDFV_______TGGGGSGGGGSGGGGSGGGGSTHTCPPCPA |
| | | PEAAGGPSVFLFPPKPKDTLMISRTPEVTCVVVDVSHEDPEVKENWYV |
| | | DGVEVHNAKTKPREEQYNSTYRVVSVLTVLHQDWLNGKEYKCKVSNKA |
| | | LPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNVF |
| | | SCSVMHEALHNHYTQKSLSLSPG |
| 115 | Alk6-Fc fusion | KKEDGESTAPTPRPKVLRCYCSGSCPEDSVNNICSTDGYCFTMIEE |
| | polypeptide | DDSGLPVVTSGCLGLEGSDFQCRDTPIPHQRRSIECCTERNECNKD |
| | K20Y, H22S, | LHPTLPPLKNRDEV_______TGGGGSGGGGSGGGGSGGGGSTHTC |
| | H23G, H24S | PPCPAPEAAGGPSVELFPPKPKDTLMISRTPEVTCVVVDVSHEDPE |
| | (i.e., Xaa₂₀ is | VKFNWYVDGVEVHNAKTKPREEQYNSTYRVVSVLTVLHQDWLNGKE |
| | Y, Xaa₂₂ is S | YKCKVSNKALPAPIEKTISKAKGQPREPQVYTLPPSREEMTKNQVS |
| | Xaa₂₃ is G, Xaa₂₄ | LTCLVKGFYPSDIAVEWESNGQPENNYKTTPPVLDSDGSFFLYSKL |
| | is S) | TVDKSRWQQGNVFSCSVMHEALHNHYTQKSLSLSPG |
| 116 | Alk6-Fc fusion | KKEDGESTAPTPRPKVLRCYCGAGHCPEDSVNNICSTDGYCFTMIE |
| | polypeptide | EDDSGLPVVTSGCLGLEGSDFQCRDTPIPHQRRSIECCTERNECNK |
| | K20Y, H22G, | DLHPTLPPLKNRDFV_______TGGGGSGGGGSGGGGSGGGGSTHT |
| | _22′A (inserted | CPPCPAPEAAGGPSVFLFPPKPKDTLMISRTPEVTCVVVDVSHEDP |
| | between aa22 and | EVKFNWYVDGVEVHNAKTKPREEQYNSTYRVVSVLTVLHQDWLNGK |
| | aa23), H23G | EYKCKVSNKALPAPIEKTISKAKGQPREPQVYTLPPSREEMTKNQV |
| | (i.e., Xaa₂₀ is | SLTCLVKGFYPSDIAVEWESNGQPENNYKTTPPVLDSDGSFFLYSK |
| | Y, Xaa₂₂ is G | LTVDKSRWQQGNVFSCSVMHEALHNHYTQKSLSLSPG |
| | _aa_{22′} is A, | |
| | Xaa₂₃ is G) | |
| 117 | Alk6-Fc fusion | KKEDGESTAPTPRPKVLRCYCGGGHCPEDSVNNICSTDGYCFTMIE |
| | polypeptide | EDDSGLPVVTSGCLGLEGSDFQCRDTPIPHQRRSIECCTERNECNK |
| | K20Y, H22G, | DLHPTLPPLKNRDFV_______TGGGGSGGGGSGGGGSGGGGSTHT |
| | _22′G (inserted | CPPCPAPEAAGGPSVFLFPPKPKDTLMISRTPEVTCVVVDVSHEDP |
| | between aa22 and | EVKFNWYVDGVEVHNAKTKPREEQYNSTYRVVSVLTVLHQDWLNGK |
| | aa23), H23G | EYKCKVSNKALPAPIEKTISKAKGQPREPQVYTLPPSREEMTKNQV |
| | (i.e., Xaa₂₀ is | SLTCLVKGFYPSDIAVEWESNGQPENNYKTTPPVLDSDGSFFLYSK |
| | Y, Xaa₂₂ is G | LTVDKSRWQQGNVFSCSVMHEALHNHYTQKSLSLSPG |
| | _aa_{22′} is G, | |
| | Xaa₂₃ is G) | |
| 118 | ALK6-Fc fusion | KKEDGESTAPTPRPKVLRCVCEGLSCPEDSVNNICSTDGYCFTMIE |
| | polypeptide | EDDSGLPVVTSGCLGLEGSDFQCRDTPIPHQRRSIECCTERNECNK |
| | K20V, H22E, | DLHPTLPPLKNRDEV_______TGGGGSGGGGSGGGGSGGGGSTHT |
| | _22′G (inserted | CPPCPAPEAAGGPSVFLFPPKPKDTLMISRTPEVTCVVVDVSHEDP |
| | between aa22 and | EVKFNWYVDGVEVHNAKTKPREEQYNSTYRVVSVLTVLHQDWLNGK |
| | aa23), H23L, and | EYKCKVSNKALPAPIEKTISKAKGQPREPQVYTLPPSREEMTKNQV |
| | H24S (i.e., | SLTCLVKGFYPSDIAVEWESNGQPENNYKTTPPVLDSDGSFFLYSK |
| | Xaa₂₀ is V, Xaa₂₂ | LTVDKSRWQQGNVFSCSVMHEALHNHYTQKSLSLSPG |
| | is E _aa_{22′} is G, | |
| | Xaa₂₃ is L, Xaa₂₄ | |
| | is S) | |
| 119 | ALK6-Fc fusion | KKEDGESTAPTPRPKVLRCVCEGLSHCPEDSVNNICSTDGYCFTMI |
| | polypeptide | EEDDSGLPVVTSGCLGLEGSDFQCRDTPIPHQRRSIECCTERNECN |
| | K20V, H22E, | KDLHPTLPPLKNRDFV_______TGGGGSGGGGSGGGGSGGGGSTH |
| | 22′G (inserted | TCPPCPAPEAAGGPSVELFPPKPKDTLMISRTPEVTCVVVDVSHED |
| | between aa22 and | PEVKFNWYVDGVEVHNAKTKPREEQYNSTYRVVSVLTVLHQDWING |
| | aa23), _22″L | KEYKCKVSNKALPAPIEKTISKAKGQPREPQVYTLPPSREEMTKNQ |
| | (inserted | VSLTCLVKGFYPSDIAVEWESNGQPENNYKTTPPVLDSDGSFFLYS |
| | between _22′ and | KLTVDKSRWQQGNVFSCSVMHEALHNHYTQKSLSLSPG |
| | aa23) H23S (i.e., | |
| | Xaa₂₀ is V, Xaa₂₂ | |
| | is E _aa_{22′} is G, | |
| | _aa_{22″} is L, Xaa₂₃ | |
| | is S) | |
| 120 | ALK6-Fc fusion | KKEDGESTAPTPRPKVLRCTCHHHCPEDSVNNICSTDGYCFTMIEE |
| | polypeptide | DDSGLPVVTSGCLGLEGSDFQCRDTPIPHQRRSIECCTERNECNKD |
| | K20T (i.e., | LHPTLPPLKNRDFV_______TGGGGSGGGGSGGGGSGGGGSTHTC |
| | Xaa₂₀ is T) | PPCPAPEAAGGPSVELFPPKPKDTLMISRTPEVTCVVVDVSHEDPE |
| | | VKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPG |
| 121 | ALK6-Fc fusion | KKEDGESTAPTPRPKVLRCVCHHHCPEDSVNNICSTDGYCFTMIEE |
| | polypeptide | DDSGLPVVTSGCLGLEGSDFQCRDTPIPHQRRSIECCTERNECNKD |
| | K20V (i.e., | LHPTLPPLKNRDEV_______TGGGGSGGGGSGGGGSGGGGSTHTC |
| | Xaa₂₀ is V) | PPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPG |
| 122 | ALK6-Fc fusion | KKEDGESTAPTPRPKVLRCFCHHHCPEDSVNNICSTDGYCFTMIEE |
| | polypeptide | DDSGLPVVTSGCLGLEGSDFQCRDTPIPHQRRSIECCTERNECNKD |
| | K20F (i.e., | LHPTLPPLKNRDFV_______TGGGGSGGGGSGGGGSGGGGSTHTC |
| | Xaa₂₀ is F) | PPCPAPEAAGGPSVFLFPPKPKDTLMISRTPEVTCVVVDVSHEDPE |
| | | VKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPG |
| 123 | ALK6-Fc fusion | KKEDGESTAPTPRPKVLRCACHHHCPEDSVNNICSTDGYCFTMIEE |
| | polypeptide | DDSGLPVVTSGCLGLEGSDFQCRDTPIPHQRRSIECCTERNECNKD |
| | K20A (i.e., | LHPTLPPLKNRDFV_______TGGGGSGGGGSGGGGSGGGGSTHTC |
| | Xaa₂₀ is A) | PPCPAPEAAGGPSVELFPPKPKDTLMISRTPEVTCVVVDVSHEDPE |
| | | VKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPG |
| 124 | ALK6-Fc fusion | KKEDGESTAPTPRPKVLRCVCGSHCPEDSVNNICSTDGYCFTMIEE |
| | polypeptide | DDSGLPVVTSGCLGLEGSDFQCRDTPIPHQRRSIECCTERNECNKD |
| | K20V, H22G, H23S | LHPTLPPLKNRDFV_______TGGGGSGGGGSGGGGSGGGGSTHTC |
| | (i.e., Xaa₂₀ is | PPCPAPEAAGGPSVFLFPPKPKDTLMISRTPEVTCVVVDVSHEDPE |
| | V, Xaa₂₂ is G, | VKFNWYVDGVEVHNAKTKPREEQYNSTYRVVSVLTVLHQDWLNGKE |
| | Xaa₂₃ is S) | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPG |
| 125 | ALK6-Fc fusion | KKEDGESTAPTPRPKVLRCFCGSHCPEDSVNNICSTDGYCFTMIEE |
| | polypeptide | DDSGLPVVTSGCLGLEGSDFQCRDTPIPHQRRSIECCTERNECNKD |
| | K20F, H22G, H23S | LHPTLPPLKNRDFV_______TGGGGSGGGGSGGGGSGGGGSTHTC |
| | (i.e., Xaa₂₀ is | PPCPAPEAAGGPSVFLFPPKPKDTLMISRTPEVTCVVVDVSHEDPE |
| | F, Xaa₂₂ is G, | VKFNWYVDGVEVHNAKTKPREEQYNSTYRVVSVLTVLHQDWLNGKE |
| | Xaa₂₃ is S) | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPG |
| 126 | ALK6-Fc fusion | KKEDGESTAPTPRPKVLRCACGSHCPEDSVNNICSTDGYCFTMIEE |
| | polypeptide | DDSGLPVVTSGCLGLEGSDFQCRDTPIPHQRRSIECCTERNECNKD |
| | K20A, H22G, H23S | LHPTLPPLKNRDEV_______TGGGGSGGGGSGGGGSGGGGSTHTC |
| | (i.e., Xaa₂₀ is | PPCPAPEAAGGPSVFLFPPKPKDTLMISRTPEVTCVVVDVSHEDPE |
| | A, Xaa₂₂ is G, | VKFNWYVDGVEVHNAKTKPREEQYNSTYRVVSVLTVLHQDWINGKE |
| | Xaa₂₃ is S) | YKCKVSNKALPAPIEKTISKAKGQPREPQVYTLPPSREEMTKNQVS |
| | | LTCLVKGFYPSDIAVEWESNGQPENNYKTTPPVLDSDGSFFLYSKL |
| | | TVDKSRWQQGNVFSCSVMHEALHNHYTQKSLSLSPG |
| 127 | ALK6-Fc fusion | KKEDGESTAPTPRPKVLRCYCGSLHCPEDSVNNICSTDGYCFTMIE |
| | polypeptide | EDDSGLPVVTSGCLGLEGSDFQCRDTPIPHQRRSIECCTERNECNK |
| | K20Y, H22G, | DLHPTLPPLKNRDEV_______TGGGGSGGGGSGGGGSGGGGSTHT |
| | _22′S (inserted | CPPCPAPEAAGGPSVFLFPPKPKDTLMISRTPEVTCVVVDVSHEDP |
| | between aa22 and | EVKFNWYVDGVEVHNAKTKPREEQYNSTYRVVSVLTVLHQDWLNGK |
| | aa23), | EYKCKVSNKALPAPIEKTISKAKGQPREPQVYTLPPSREEMTKNQV |
| | _H23L(i.e., | SLTCLVKGFYPSDIAVEWESNGQPENNYKTTPPVLDSDGSFFLYSK |
| | Xaa₂₀ is Y, Xaa₂₂ | LTVDKSRWQQGNVFSCSVMHEALHNHYTQKSLSLSPG |
| | is G _aa_{22′} is S, | |
| | _Xaa₂₃ is L) | |
| 128 | ALK6-Fc fusion | KKEDGESTAPTPRPKVLRCYCGSGHCPEDSVNNICSTDGYCFTMIE |
| | polypeptide | EDDSGLPVVTSGCLGLEGSDFQCRDTPIPHQRRSIECCTERNECNK |
| | K20Y, H22G, | DLHPTLPPLKNRDFV_______TGGGGSGGGGSGGGGSGGGGSTHT |
| | _22′S (inserted | CPPCPAPEAAGGPSVELFPPKPKDTLMISRTPEVTCVVVDVSHEDP |
| | between aa22 and | EVKFNWYVDGVEVHNAKTKPREEQYNSTYRVVSVLTVLHQDWLNGK |
| | aa23), | EYKCKVSNKALPAPIEKTISKAKGQPREPQVYTLPPSREEMTKNQV |
| | _H23G (i.e., | SLTCLVKGFYPSDIAVEWESNGQPENNYKTTPPVLDSDGSFFLYSK |
| | Xaa₂₀ is Y, Xaa₂₂ | LTVDKSRWQQGNVFSCSVMHEALHNHYTQKSLSLSPG |
| | is G _aa_{22′} is S, | |
| | _Xaa₂₃ is G) | |
| 129 | ALK6-Fc fusion | KKEDGESTAPTPRPKVLRCYCSGHCPEDSVNNICSTDGYCFTMIEE |
| | polypeptide | DDSGLPVVTSGCLGLEGSDFQCRDTPIPHQRRSIECCTERNECNKD |
| | K20Y, H22S, | LHPTLPPLKNRDEV_______TGGGGSGGGGSGGGGSGGGGSTHTC |
| | _H23G (i.e., | PPCPAPEAAGGPSVELFPPKPKDTLMISRTPEVTCVVVDVSHEDPE |
| | Xaa₂₀ is Y, Xaa₂₂ | VKFNWYVDGVEVHNAKTKPREEQYNSTYRVVSVLTVLHQDWLNGKE |
| | is S, Xaa₂₃ is | YKCKVSNKALPAPIEKTISKAKGQPREPQVYTLPPSREEMTKNQVS |
| | G) | LTCLVKGFYPSDIAVEWESNGQPENNYKTTPPVLDSDGSFFLYSKL |
| | | TVDKSRWQQGNVFSCSVMHEALHNHYTQKSLSLSPG |
| 130 | SEQ ID NO: 108 | Lys₁ - Lys₂ - Glu₃ - Asp₄ - Gly₅ - Glu₆ - Ser₇ - Thr₈ - |
| | with addition of | Alag - Pro₁₀ - Thr₁₁ - Pro₁₂ - Arg₁₃ - Pro₁₄ - Lys₁₅ - Val₁₆ - |
| | aaA between ′22 | Leu₁₇ - Arg₁₈ - Cys₁₉ - Xaa₂₀ - Cys₂₁ - Xaa₂₂ - Xaa′₂₂ - |
| | and 23 | Xaa″₂₂ - Xaa₂₃ - Xaa₂₄ - Cys₂₅ - Pro₂₆ - Xaa₂₇ - Asp₂₈ - |
| | | Xaa₂₉ - Xaa₃₀ - Asn₃₁ - Asn₃₂ - Xaa₃₃ - Cys₃₄ - Xaa₃₅ - |
| | | Thr₃₆ - Xaa₃₇ - Gly₃₈ - Xaa₃₉ - Cys₄₀ - Phe₄₁ - Xaa₄₂ - |
| | | Xaa₄₃ - Ile₄₄ - Glu₄₅ - Glu₄₆ - Asp₄₇ - Asp₄₈ - Xaa₄₉ - |
| | | Gly₅₀ - Xaa₅₁ - Xaa₅₂ - Xaa₅₃ - Xaa₅₄ - Xaa₅₅ - Ser₅₆ - |
| | | Gly₅₇ - Cys₅₈ - Xaa₅₉ - Xaa₆₀ - Xaa₆₁ - Glu₆₂ - Gly₆₃ - |
| | | Ser₆₄ - Asp₆₅ - Phe₆₆ - Gln₆₇ - Cys₆₈ - Xaa₆₉ - Asp₇₀ - |
| | | Xaa₇₁ - Pro₇₂ - Xaa₇₃ - Xaa₇₄ - Xaa₇₅ - Xaa₇₆ - Arg₇₇ - |
| | | Arg₇₈ - Xaa₇₉ - Ile₈₀ - Glu₈₁ - Cys₈₂ - Cys₈₃ - Xaa₈₄ - Xaa₈₅ - |
| | | Xaa₈₆ - Xaa₈₇ - Xaa₈₈ - Cys₈₉ - Asn₉₀ - Xaa₉₁ - Xaa₉₂ - |
| | | Leu₉₃ - Xaa₉₄ - Pro₉₅ - Thr₉₆ - Leu₉₇ - Pro₉₈ - Pro₉₉ - |
| | | Leu₁₀₀ - Lys₁₀₁ - Asn₁₀₂ - Arg₁₀₃ - Asp₁₀₄ - Phe₁₀₅ - Val₁₀₆ - |
| | | Xaa₁₀₇ - Xaa₁₀₈ - Xaa₁₀₉ - Xaa₁₁₀ - Xaa₁₁₁ - Xaa₁₁₂ - |
| | | Xaa₁₁₃; |
| | | wherein Xaa₂₀ is V, F, K, Y, T, or A; |
| | | Xaa₂₂ is G, E, H or S; |
| | | Xaa′₂₂ is absent, G, S, or A; |
| | | Xaa″₂₂ is absent or S; |
| | | Xaa₂₃ is S, H, G, or L; |
| | | Xaa₂₄ is H or S; |
| | | Xaa₂₇ is E or D; |
| | | Xaa₂₉ is S or A; |
| | | Xaa₃₀ is V or I; |
| | | Xaa₃₃ is I or T; |
| | | Xaa₃₅ is S or I; |
| | | Xaa₃₇ is D or N; |
| | | Xaa₃₉ is Y or H; |
| | | Xaa₄₂ is T or A; |
| | | Xaa₄₃ is M or I; |
| | | Xaa₄₉ is S or Q; |
| | | Xaa₅₁ is L or E; |
| | | Xaa₅₂ is P or T; |
| | | Xaa₅₃ is V or T; |
| | | Xaa₅₄ is V or L; |
| | | Xaa₅₅ is T or A; |
| | | Xaa₅₉ is L or M; |
| | | Xaa₆₀ is G or K; |
| | | Xaa₆₁ is L or Y; |
| | | Xaa₆₉ is R or K; |
| | | Xaa₇₀ is T or S; |
| | | Xaa₇₁ is I or K; |
| | | Xaa₇₄ is P or A; |
| | | Xaa₇₅ is H or Q; |
| | | Xaa₇₆ is Q or L; |
| | | Xaa₇₉ is S or T; |
| | | Xaa₈₄ is T or R; |
| | | Xaa₈₅ is absent, T or E; |
| | | Xaa₈₆ is R or N; |
| | | Xaa₈₇ is N or L; |
| | | Xaa₈₈ is absent, E or L; |
| | | Xaa₉₁ is K or Q; |
| | | Xaa₉₂ is D or L; |
| | | Xaa₉₄ is H or Q; |
| | | Xaa₁₀₇ is absent; |
| | | Xaa₁₀₈ is absent; |
| | | Xaa₁₀₉ is absent; |
| | | Xaa₁₁₀ is absent; |
| | | Xaa₁₁ is absent; |
| | | Xaa₁₁₂ is absent; |
| | | and, Xaa₁₁₃ is absent |
| 131 | ALK3 ECD + liker | QNLDSMLHGTGMKSDSDQKKSENGVTLAPEDTLPFLK |
| | + Fc Sequence | CYCSGHCPDDAINNTCITNGHCFAIIEEDDQGETTLA |
| | | SGCMKYEGSDFQCKDSPKAQLRRTIECCRTNLCNQYL |
| | | QPTLPPVVIGPFFDGSIRTGGGGSGGGGSGGGGSGGG |
| | | GSTHTCPPCPAPELLGGPSVELFPPKPKDTLMISRTP |
| | | EVTCVVVDVSHEDPEVKENWYVDGVEVHNAKTKPREE |
| | | QYNSTYRVVSVLTVLHQDWLNGKEYKCKVSNKALPAP |
| | | IEKTISKAKGQPREPQVYTLPPSREEMTKNQVSLTCL |
| | | VKGFYPSDIAVEWESNGQPENNYKTTPPVLDSDGSFF |
| | | LYSKLTVDKSRWQQGNVFSCSVMHEALHNHYTQKSLS |
| | | LSPGK |

### EXAMPLES

The following examples are provided to illustrate certain disclosed embodiments and are not to be construed as limiting the scope of this disclosure in any way.

### Example 1: Generation of ALK-6 Fc Fusion Proteins

Applicants constructed a soluble ALK6wt-Fc dimeric complex comprising the extracellular domain (ECD) of human ALK6 (SEQ ID NO: 1) wherein the ECD is fused to an Fc domain with a linker positioned between the extracellular domain and the Fc domain. The individual constructs are referred to as ALK6wt-Fc fusion polypeptide, the sequence of which is provided below. In addition, the ALK6-Fc fusion polypeptide employs the leader sequence:

SEQ ID NO: 9
MGWSCIILFLVATATGVHS

The ALK6wt-Fc polypeptide sequence (SEQ ID NO: 49) is shown below:

MGWSCIILFLVATATGVHSKKEDGESTAPTPRPKVLRCKCHHHCPEDSVN
NICSTDGYCFTMIEEDDSGLPVVTSGCLGLEGSDFQCRDTPIPHQRRSIE
CCTERNECNKDLHPTLPPLKNRDFVDGPIHHRTGGGGSGGGGSGGGGSGG
GGSTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGK

The leader (signal) sequence and linker are underlined. The amino acid sequence of SEQ ID NO: 49 may optionally be provided with lysine (K) removed from the C-terminus.

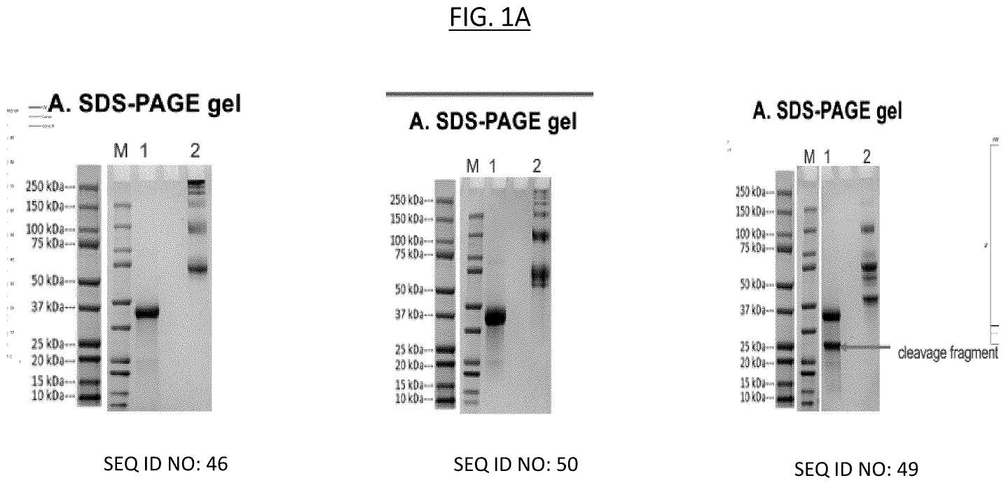

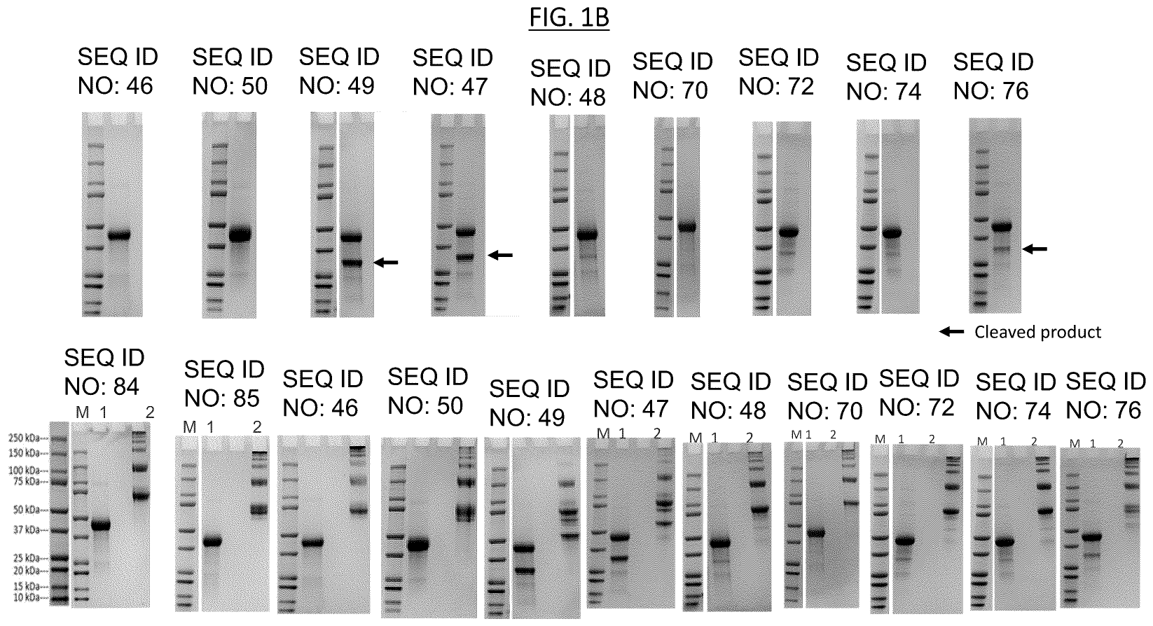

SDS gel analysis of SEQ ID NO: 49 shows a prominent cleavage fragment at about 25 kDa. See FIG. 1. Based on the fragment size and analysis of the sequence a protease cleavage site in the C-terminus end of the ECD of ALK6 was indicated.

To generate a non-cleavable soluble ALK6 Fc polypeptide, Applicants constructed a soluble ALK-6 Fc fusion polypeptide wherein the C-terminus 14 amino acids (SEQ ID NO: 51; LKNRDFVDGPIHHR) were deleted from the ECD of human wild type ALK6 providing SEQ ID NO: 4. The ECD was fused to an Fc domain with a linker positioned between the ECD and the Fc domain. The individual constructs are referred to as ALK6-Fc fusion polypeptide (SEQ ID NO: 50); the fusion polypeptide sequence of which is provided below.

MGWSCIILFLVATATGVHSKKEDGESTAPTPRPKVLRCKCHHHCPEDSVN
NICSTDGYCFTMIEEDDSGLPVVTSGCLGLEGSDFQCRDTPIPHQRRSIE
CCTERNECNKDLHPTLPPLTGGGGSGGGGSGGGGSGGGGSTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK

The leader (signal) sequence and linker are underlined. The amino acid sequence of SEQ ID NO: 50 may optionally be provided with lysine (K) removed from the C-terminus.

SDS gel analysis of SEQ ID NO: 50 showed no cleave fragment as compared to SEQ ID NO: 49. See FIG. 1.

In another approach to generate a non-cleavable ALK6-Fc fusion polypeptide, Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR). The ALK6 ECD (SEQ ID NO: 11) was fused to a Fc domain with a linker positioned between the ECD and the Fc domain. The individual constructs are referred to as ALK6-Fc fusion polypeptide (SEQ ID NO: 46), the fusion polypeptide sequence of which is provided below.

KKEDGESTAPTPRPKVLRCKCHHHCPEDSVNNICSTDGYCFTMIEEDDSG
LPVVTSGCLGLEGSDFQCRDTPIPHQRRSIECCTERNECNKDLHPTLPPV
VIGPFFDGSIRTGGGGSGGGGSGGGGSGGGGSTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The linker sequence is underlined, and the new C-terminus sequence in bold. The amino acid sequence of SEQ ID NO: 46 may optionally be provided with lysine (K) removed from the C-terminus and/or with a leader sequence.

SDS gel analysis of SEQ ID NO: 46 showed no cleave fragment as compared to SEQ ID NO: 49. See FIG. 1.

In another approach to generate a non-cleavable ALK6-Fc fusion polypeptide and maintain the ALK6 ligand binding properties, Applicants constructed a soluble ALK-6 Fc fusion polypeptide wherein a point mutation was introduced into the suspected cleavage site with the mutation D104A introduced into the SEQ ID NO: 1 sequence providing SEQ ID NO: 12. The ECD was fused to a Fc domain with a linker positioned between the ECD and the Fc domain. The individual constructs are referred to as a ALK6-Fc fusion polypeptide (SEQ ID NO: 47), the fusion polypeptide sequence of which is provided below.

KKEDGESTAPTPRPKVLRCKCHHHCPEDSVNNICSTDGYCFTMIEEDDSG
LPVVTSGCLGLEGSDFQCRDTPIPHQRRSIECCTERNECNKDLHPTLPPL
KNRAFVDGPIHHRTGGGGSGGGGGGGGSGGGGSTHTCPPCPAPELLGGPS
VFLFPPKPKDTLMISRTPEVTCVVVDVSHEDPEVKFWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The linker sequence and the individual point mutation in the ALK6 ECD are underlined and bold. The amino acid sequence of SEQ ID NO: 47 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein a point mutation was introduced into the suspected cleavage site with the mutation D104A introduced into the SEQ ID NO: 1 sequence and seven amino acids deleted from the C-terminus providing SEQ ID NO: 13. The ALK6 ECD was fused to a Fc domain with a linker positioned between the ECD and the Fc domain. The individual constructs are referred to as an ALK6-Fc fusion polypeptide (SEQ ID NO: 48), the fusion polypeptide sequence of which is provided below,

KKEDGESTAPTPRPKVLRCKCHHHCPEDSVNNICSTDGYCFTMIEEDDSG
LPVVTSGCLGLEGSDFQCRDTPIPHORRSIECCTERNECNKDLHPTLPPL
KNRAFV TGGGGSGGGGSGGGGSGGGGSTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK

The linker sequence and the individual point mutation in the ALK6 ECD are underlined and bold. The amino acid sequence of SEQ ID NO: 48 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein seven amino acids were deleted from the C-terminus of ALK-6 ECD of SEQ ID NO: 1 providing SEQ ID NO:69. The ALK6 ECD was fused to a Fc domain with a linker positioned between the ECD and the Fc domain. The individual constructs are referred to as a ALK6-Fc fusion polypeptide (SEQ ID NO: 70), the fusion polypeptide sequence of which is provided below.

KKEDGESTAPTPRPKVLRCKCHHHCPEDSVNNICSTDGYCFTMIEEDDSG
LPVVTSGCLGLEGSDFQCRDTPIPHQRRSIECCTERNECNKDLHPTLPPL
KNRDFV TGGGGSGGGGSGGGGSGGGGSTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK

The linker sequence and the deletion mutations in the ALK6 ECD are underlined and bold. The amino acid sequence of SEQ ID NO: 70 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein a point mutation was introduced into the suspected cleavage site with the mutation D107A introduced into the SEQ ID NO: 1 sequence providing SEQ ID NO: 71. The ECD was fused to a Fc domain with a linker positioned between the ECD and the Fc domain. The individual constructs are referred to as anALK6-Fc fusion polypeptide (SEQ ID NO: 72) the fusion polypeptide sequence of which is provided below.

KKEDGESTAPTPRPKVLRCKCHHHCPEDSVNNICSTDGYCFTMIEEDDSG
LPVVTSGCLGLEGSDFQCRDTPIPHQRRSIECCTERNECNKDLHPTLPPL
KNRDFVAGPIHHRTGGGGSGGGGSGGGGSGGGGSTHTCPPCPAPELLGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPGK

The linker sequence and the point mutation in the ALK6 ECD are underlined and bold. The amino acid sequence of SEQ ID NO: 72 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein a point mutation was introduced into the suspected cleavage site with the mutation D107A introduced into the SEQ ID NO: 1 sequence and two amino acids deleted (H111_ and H112_) from the C-terminus providing SEQ ID NO: 73. The ECD was fused to a Fc domain with a linker positioned between the ECD and the Fc domain. The individual constructs are referred to as ALK6-Fc fusion polypeptide (SEQ ID NO: 74, the fusion polypeptide sequence of which is provided below.

KKEDGESTAPTPRPKVLRCKCHHHCPEDSVNNICSTDGYCFTMIEEDDSG
LPVVTSGCLGLEGSDFQCRDTPIPHQRRSIECCTERNECNKDLHPTLPPL
KNRDFVAGPI RTGGGGSGGGGSGGGGSGGGGSTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPGK

The linker sequence and the point and deletion mutations in the ALK6 ECD are underlined and bold. The amino acid sequence of SEQ ID NO: 74 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein a point mutation was introduced into the suspected cleavage site with the mutation P109S introduced into the SEQ ID NO: 1 sequence and two amino acids deleted (H112_ and H113_) from the C-terminus providing SEQ ID NO: 75. The ECD was fused to a Fc domain with a linker positioned between the ECD and the Fc domain. The individual constructs are referred to as an ALK6-Fc fusion polypeptide (SEQ ID NO: 76), the fusion polypeptide sequence of which is provided below.

KKEDGESTAPTPRPKVLRCKCHHHCPEDSVNNICSTDGYCFTMIEEDDSG
LPVVTSGCLGLEGSDFQCRDTPIPHQRRSIECCTERNECNKDLHPTLPPL
KNRDFEVDGSI RTGGGGSGGGGSGGGGSGGGGSTHTCPPCPAPELLGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPGK

The linker sequence and the point and deletion mutations in the ALK6 ECD are underlined and bold. The amino acid sequence of SEQ ID NO: 76 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein an additional amino acid was introduced between position 22 and 23 with the mutation _22′A introduced into the SEQ ID NO: 1 sequence and seven amino acids were deleted from the C-terminus of ALK-6 ECD of SEQ ID NO: 1 providing SEQ ID NO:113. The ECD was fused to a Fc domain with a linker positioned between the ECD and the Fc domain. The individual constructs are referred to as an ALK6-Fc fusion polypeptide (SEQ ID NO: 114), the fusion polypeptide sequence of which is provided below.

KKEDGESTAPTPRPKVLRCKCHAHHCPEDSVNNICSTDGYCFTMIEEDDS
GLPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPG

The linker sequence and the point and deletion mutations in the ALK6 ECD are underlined and bold as are the two point mutations in the Fc sequence. The amino acid sequence of SEQ ID NO: 114 may optionally be provided with lysine (K) added to the C-terminus and/or with a leader sequence.

### Example 2: Generation of Alternative ALK-6 Fc Fusion Proteins

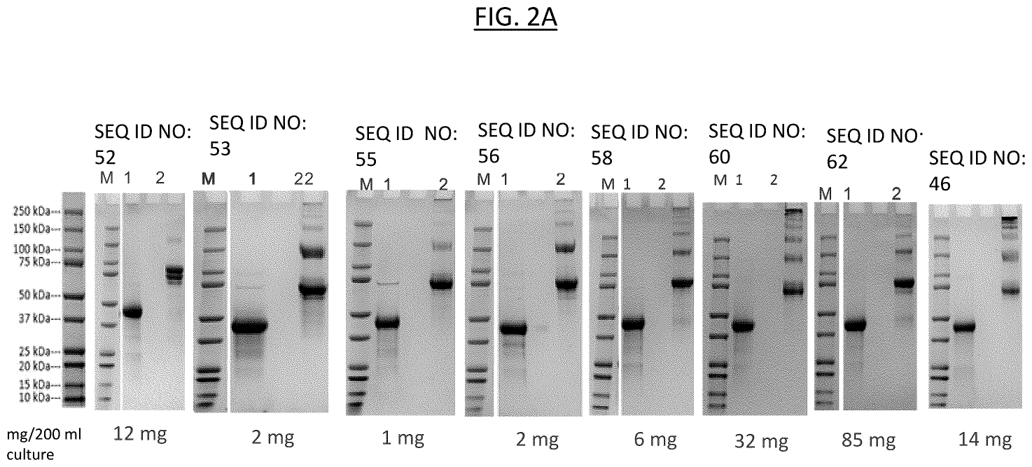

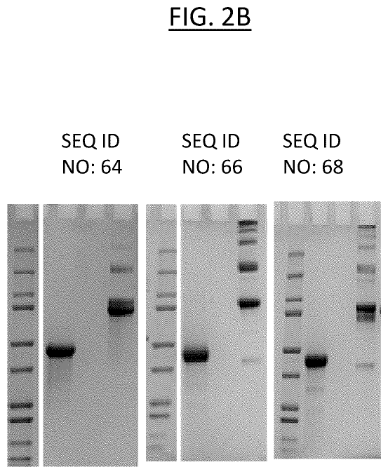

Applicants constructed a number of soluble ALK-6 Fc fusion polypeptides using SEQ ID NO: 46 as a background sequence introducing one or more point mutations in the modified ECD of ALK6 (“SEQ ID NO:11 variants”). These variant soluble ALK-6 Fc fusion polypeptides were analyzed on a SDS gel to confirm the lack of a cleavage product and select variants tested in a Biacore-based binding assay for ligand binding specificity. See FIGS. 2 and 3.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutation H23G was introduced into the modified ECD of ALK6 providing SEQ ID NO:20. The ECD was fused to a Fc domain with a linker positioned between the ECD and the Fc domain. The individual constructs are referred to as an ALK6-Fc fusion polypeptide (SEQ ID NO: 52), the fusion polypeptide sequence of which is provided below.

KKEDGESTAPTPRPKVLRCKCHGHCPEDSVNNICSTDGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutation in the ALK6 ECD are underlined and bold. The amino acid sequence of SEQ ID NO: 52 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutation D37N was introduced into the modified ECD of ALK6 providing SEQ ID NO:26. The ECD was fused to a Fc domain with a linker positioned between the ECD and the Fc domain. The individual constructs are referred to as ALK6-Fc fusion polypeptide (SEQ ID NO: 53), the fusion polypeptide sequence of which is provided below,

KKEDGESTAPTPRPKVLRCKCHHHCPEDSVNNICSTNGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutation in the ALK6 ECD are underlined and bold. The amino acid sequence of SEQ ID NO: 53 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations T42A and M43I were introduced into the modified ECD of ALK6 providing SEQ ID NO:28. The ECD was fused to a Fc domain with a linker positioned between the ECD and the Fc domain. The individual constructs are referred to as an ALK6-Fc fusion polypeptide (SEQ ID NO: 54), the fusion polypeptide sequence of which is provided below.

KKEDGESTAPTPRPKVLRCKCHHHCPEDSVNNICSTDGYCFAIIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutations in the ALK6 ECD are underlined and bold. The amino acid sequence of SEQ ID NO: 54 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations L59M, G60K and L61Y were introduced into the modified ECD of ALK6 providing SEQ ID NO:33. The ECD was fused to a Fc domain with a linker positioned between the ECD and the Fc domain. The individual constructs are referred to as ALK6-Fc fusion polypeptide (SEQ ID NO: 55), the fusion polypeptide sequence of which is provided below.

KKEDGESTAPTPRPKVLRCKCHHHCPEDSVNNICSTDGYCFTMIEEDDS
GLPVVTSGCMKYEGSDFQCRDTPIPHQRRSIECCTERNECNKDLHPTLP
PVVIGPFFDGSIRTGGGGSGGGGSGGGGGGGGSTHTCPPCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHN
HYTQKSLSLSPGK

The linker sequence and the individual point mutations in the ALK6 ECD are underlined and bold. The amino acid sequence of SEQ ID NO: 55 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations G60K, I73K, P74A, H75Q and Q76L were introduced into the modified ECD of ALK6 providing SEQ ID NO:41. The ECD was fused to a Fc domain with a linker positioned between the ECD and the Fc domain. The individual constructs are referred to as ALK6-Fc fusion polypeptide (SEQ ID NO: 56), the fusion polypeptide sequence of which is provided below.

KKEDGESTAPTPRPKVLRCKCHHHCPEDSVNNICSTDGYCFTMIEEDDS
GLPVVTSGCLKLEGSDFQCRDTPKAQLRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutations in the ALK6 ECD are underlined and bold. The amino acid sequence of SEQ ID NO: 56 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutation H22S was introduced into the modified ECD of ALK6 providing SEQ ID NO:57. The ECD was fused to a Fc domain with a linker positioned between the ECD and the Fc domain. The individual constructs are referred to as ALK6-Fc fusion polypeptide (SEQ ID NO: 58), the fusion polypeptide sequence of which is provided below,

KKEDGESTAPTPRPKVLRCKCSHHCPEDSVNNICSTDGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutations in the ALK6 ECD are underlined and bold. The amino acid sequence of SEQ ID NO: 58 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations H22S and H23G were introduced into the modified ECD of ALK6 providing SEQ ID NO:59. The ECD was fused to a Fc domain with a linker positioned between the ECD and the Fc domain. The individual constructs are referred to as ALK6-Fc fusion polypeptide (SEQ ID NO: 60), the fusion polypeptide sequence of which is provided below.

KKEDGESTAPTPRPKVLRCKCSGHCPEDSVNNICSTDGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutations in the ALK6 ECD are underlined and bold. The amino acid sequence of SEQ ID NO: 60 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations K20Y, H22S and H23G were introduced into the modified ECD of ALK6 providing SEQ ID NO:61. The ECD was fused to a Fc domain with a linker positioned between the ECD and the Fc domain. The individual constructs are referred to as an ALK6-Fc fusion polypeptide (SEQ ID NO: 62), the fusion polypeptide sequence of which is provided below.

KKEDGESTAPTPRPKVLRCYCSGHCPEDSVNNICSTDGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutations in the ALK6 ECD are underlined and bold. The amino acid sequence of SEQ ID NO: 62 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations K20Y and H23G were introduced into the modified ECD of ALK6 providing SEQ ID NO:63. The ECD was fused to a Fc domain with a linker positioned between the ECD and the Fc domain. The individual constructs are referred to as an ALK6-Fc fusion polypeptide (SEQ ID NO: 64), the fusion polypeptide sequence of which is provided below,

KKEDGESTAPTPRPKVLRCYCHGHCPEDSVNNICSTDGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutations in the ALK6 ECD are underlined and bold. The amino acid sequence of SEQ ID NO: 64 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutation K20Y was introduced into the modified ECD of ALK6 providing SEQ ID NO:65. The ECD was fused to a Fc domain with a linker positioned between the ECD and the Fc domain. The individual constructs are referred to as an ALK6-Fc fusion polypeptide (SEQ ID NO: 66) the fusion polypeptide sequence of which is provided below.

KKEDGESTAPTPRPKVLRCYCHHHCPEDSVNNICSTDGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutations in the ALK6 ECD are underlined and bold. The amino acid sequence of SEQ ID NO: 66 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations K20Y, H22S, H23G, and G60L was introduced into the modified ECD of ALK6 providing SEQ ID NO:88. The ECD was fused to a Fc domain with a linker positioned between the ECD and the Fc domain. The individual constructs are referred to as an ALK6-Fc fusion polypeptide (SEQ ID NO: 88), the fusion polypeptide sequence of which is provided below.

KKEDGESTAPTPRPKVLRCYCSGHCPEDSVNNICSTDGYCFTMIEEDDS
GLPVVTSGCLKLEGSDFQCRDTPIPHQRRSIECCTERNECNKDLHPTLP
PVVIGPFFDGSIRTGGGGSGGGGSGGGGSGGGGSTHTCPPCPAPEAAGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPG

The linker sequence and the individual point mutations in the ALK6 ECD and Fe sequence are underlined and bold. The amino acid sequence of SEQ ID NO: 89 may optionally be provided with lysine (K) removed (as shown) from the C-terminus, or added and/or with a leader sequence.

Applicants further constructed a soluble ALK-6 Fc fusion polypeptide using SEQ ID NO: 73 as a background sequence introducing three point mutations in the modified ECD of ALK6 (SEQ ID NO:1 variant). The variant soluble ALK-6 Fc fusion polypeptide was analyzed on a SDS get to confirm the lack of a cleavage product. See FIG. 2.

Applicants constructed a soluble ALK-6 Fc fusion polypeptide wherein the point mutation D107A was introduced and the last two histidine amino acids (H111_and H112_) were deleted from the C terminus of SEQ ID NO: 1 and wherein the point mutations K20Y, H22S and H23G were introduced into the modified ECD of ALK6 providing SEQ ID NO:67. The ECD was fused to a Fc domain with a linker positioned between the ECD and the Fc domain. The individual constructs are referred to as an ALK6-Fc fusion polypeptide (SEQ ID NO: 68), the fusion polypeptide sequence of which is provided below.

KKEDGESTAPTPRPKVLRCYCSGHCPEDSVNNICSTDGYCFTMIEEDDSGLPVV
TSGCLGLEGSDFQCRDTPIPHQRRSIECCTERNECNKDLHPTLPPLKNRDEVAGPI__RTGGGG
SGGGGSGGGGSGGGGSTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

The linker sequence and the individual point mutations in the ALK6 ECD are underlined and bold. The amino acid sequence of SEQ ID NO: 68 may optionally be provided with lysine (K) removed from the C-terminus and/or with a leader sequence.

Applicants further constructed a soluble ALK-6 Fc fusion polypeptide using SEQ ID NO: 69 as a background sequence introducing point mutations in the modified ECD of ALK6 (SEQ ID) NO: 1 variant). The variant soluble ALK-6 Fe fusion polypeptide was analyzed on a SDS gel to confirm the lack of a cleavage product and select variants tested in a Biacore-based binding assay for ligand binding specificity. Data not shown.

Applicants constructed a soluble ALK-6 Fc fusion polypeptide wherein seven amino acids from the C-terminus of SEQ ID NO: 1 were deleted and wherein the point mutations K20Y, H22S and H23G were introduced into the modified ECD of ALK6 providing SEQ ID NO:86. The ECD was fused to a Fc domain with a linker positioned between the ECD and the Fc domain. The individual constructs are referred to as an ALK6-Fc fusion polypeptide (SEQ ID NO: 87), the fusion polypeptide sequence of which is provided below.

KKEDGESTAPTPRPKVLRCYCSGHCPEDSVNNICSTDGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPEAAGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPG

The linker sequence and the individual point mutations in the ALK6 ECD and Fc sequence are underlined and bold. The amino acid sequence of SEQ ID NO: 87 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein an amino acid Arginine was added between positions 22 and 23 (_22′A) and the point mutations K20Y, H22S and H23G were introduced into the modified ECD of ALK6 providing SEQ ID NO:90. The ECD was fused to a Fc domain with a linker positioned between the ECD and the Fc domain. The individual constructs are referred to as an ALK6-Fc fusion polypeptide (SEQ ID NO: 91), the fusion polypeptide sequence of which is provided below.

KKEDGESTAPTPRPKVLRCYCSAGHCPEDSVNNICSTDGYCFTMIEEDD
SGLPVVTSGCLGLEGSDFQCRDTPIPHQRRSIECCTERNECNKDLHPTL
PPVVIGPFFDGSIRTGGGGSGGGGSGGGGSGGGGSTHTCPPCPAPEAAG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEAL
HNHYTQKSLSLSPG

The linker sequence and the individual point mutations in the ALK6 ECD and Fe sequence are underlined and bold. The amino acid sequence of SEQ ID NO: 87 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations K20Y, H22S, H23G and D37N were introduced into the modified ECD of ALK6 providing SEQ ID NO:92. The ECD was fused to a Fc domain with a linker positioned between the ECD and the Fc domain. The individual constructs are referred to as an ALK6-Fc fusion polypeptide (SEQ ID NO: 93), the fusion polypeptide sequence of which is provided below.

KKEDGESTAPTPRPKVLRCYCSGHCPEDSVNNICSTNGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPEAAGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPG

The linker sequence and the individual point mutations in the ALK6 ECD and Fe sequence are underlined and bold. The amino acid sequence of SEQ ID NO: 87 may optionally be provided with lysine (K) added or removed from the C-terminus and/or with a leader sequence.

### Example 3: In Vitro Screening to Identify ALK-6 Fc Fusion Protein with Binding to BMP4 but not BMP10

A Biacore™-based binding assay was used to compare ligand binding selectivity of the ALK6 variant-Fc dimeric complex (e.g., SEQ ID NO: 46) described above with that of ALK6 wt-Fc complexes (SEQ ID NO: 49). The Fc dimeric complexes were independently captured onto the system using an anti-Fe antibody. Ligands were injected and allowed to flow over the captured receptor protein. Results are summarized in the table below and provided in FIGS. 3A and 3B.

TABLE 1
Ligand Binding Profile of ALK6 variant-Fc dimeric complex

| BMP4 Positive Binding | BMP10 Positive Binding |
|---|---|
| ALK6wt-Fc SEQ ID NO: 49 | ALK6wt-Fc SEQ ID NO: 49 |
| SEQ ID NO: 46 | SEQ ID NO: 46 |
| SEQ ID NO: 47 | SEQ ID NO: 47 |
| SEQ ID NO: 48 | SEQ ID NO: 48 |
| SEQ ID NO: 52 | SEQ ID NO: 52 |
| SEQ ID NO: 53 | SEQ ID NO: 53 |
| SEQ ID NO: 55 | No binding |

Ligand off-rate is a particularly significant parameter to evaluate for ligand traps. Soluble receptor-Fc proteins administered in vivo are in constant competition with native receptors for ligands. When endogenous ligands of the TGFβ superfamily typically bind to cognate receptors at the cell surface, a multi-step signal transduction process is triggered that is relatively slow on a molecular time scale. Native receptors dissociate from ligand slowly in part because significant time is required to generate an intracellular signal from a ligand binding event. For a soluble receptor-Fe protein to compete effectively for ligand, the off-rate for its complex with the ligand needs to be similar to, or slower than, the off-rate for a ligand complex with native receptor. Ligand binding is a dynamic process and some fraction of ligands will always be in unbound form, so it is important therapeutically for a dose of receptor-Fe protein to capture target ligand for as long as possible. One way to shift the binding equilibrium in favor of more captured ligand is to increase the concentration (dose level) of inhibitor, however this can generate off-target effects that reduce tolerability and safety. A preferable approach is to use an inhibitor with a slower ligand off-rate (longer capture time) combined with ligand binding selectivity to achieve an effective level of ligand antagonism at a lower concentration of inhibitor.

### Example 4: Cell Based Reporter Gene Assay

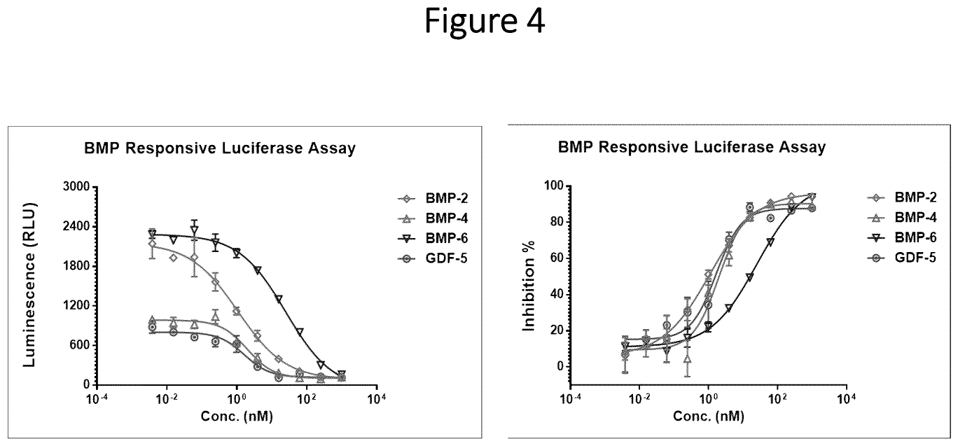

20,000 cells/well in 96-well plate were inoculated with the activation concentrations of 4 ligands (BMP-2; BMP-4; BMP-6 and GDF-5) set at their respective EC50 values (1.076 nM; 1.995 nM; 25.01 nM and 1.647 nM). The concentration of the inhibitor (SEQ ID NO: 87) was 1000 nM, with 4-fold dilution, 10 concentration points, and incubation at 37° C. for 3 hours. SEQ ID NO: 87 effectively inhibited the luciferase signals activated by the four ligands demonstrating binding consistent with Biacore data for SEQ ID NO 87. See FIG. 4.

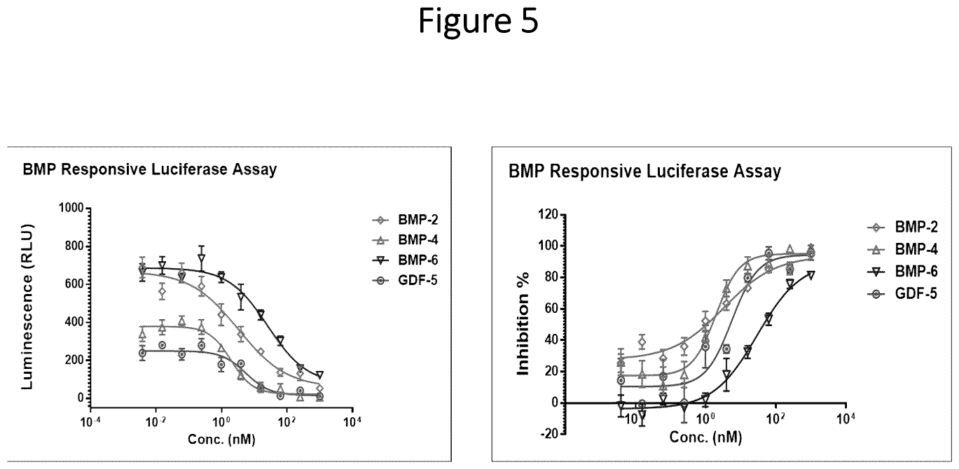

20,000 cells/well in 96-well plate were inoculated with the activation concentrations of 4 ligands (BMP-2; BMP-4; BMP-6 and GDF-5) set at their respective EC50 values (3.286 nM; 1.951 nM; 29.1 nM and 5.51 nM). The concentration of the inhibitor (SEQ ID NO: 70) was 1000 nM, with 4-fold dilution, 10 concentration points, and incubation at 37° C. for 3 hours. SEQ ID NO: 70 effectively inhibited the luciferase signals activated by the four ligands demonstrating binding consistent with Biacore data for SEQ ID NO 70. See FIG. 5.

### Example 5: Induction of EAE and Treatment with ALK-6 Fe Fusion Proteins

Experimental autoimmune encephalomyelitis (EAE) is a widely accepted animal model of demyelinating disease, including multiple sclerosis (MS). Similar to MS, EAE is characterized by infiltration of immune cells into the central nervous system (CNS) and demyelination. There are two commonly used models of EAE. One model is produced by administering a myelin basic protein peptide (MBP) fragment, typically in combination with an immune-boosting adjuvant and pertussis toxin, myelin reactive primed T-cells, or by expressing an autoimmune T-cell receptor, that induces an autoimmune response directed to the myelin sheath surrounding motor neurons. These stimuli evoke an inflammatory autoimmune response against myelin and axons, facilitated by CNS infiltrating T-cells and monocytes that cause demyelination and disability. EAE is thought to model lesion formation and inflammatory injury characteristic of relapsing-remitting MS. In particular, demyelination of neurons within the CNS leads to impaired locomotor function and mirrors symptoms of the human disease. As with multiple sclerosis in humans, the condition in rodents appears in relapsing-remitting cycles and are characterized by loss of nerve conduction and chronic progression of disability.

In this experiment, female SJL/J mice (6-7 weeks of age) were purchased from Jackson Laboratory. Animal experimentation was approved by an Institutional Animal Care and Use Committee. The studies were conducted in accordance with the United States Public Health Service's Policy on Humane Care and Use of Laboratory Animals.

R-EAE was induced by subcutaneous immunization (day 0) with 100 μg of PLP₁₃₉₋₁₅₁ (Anaspec, Inc., San Jose, CA) in 0.1 ml of PBS per mouse. The peptide was emulsified in an equal volume of complete Freund's Adjuvant (CFA, Rockland, Gilbertsville, PA) containing 500 μg of Mycobacterium tuberculosis H37RA (BD, ATCC, Mannassas VA). The mice subsequently received on Day 1 (24 hours) and Day 2 (48 hours) an intraperitoneal (IP) injection of 300 ng/mouse of pertussis toxin (PTX, from List Biological Labs) in 0.2 ml of PBS. The mice were randomly divided into 3 groups with 8 mice per group and starting on Day 3 mice were dosed with A) Gilenya (0.1 mg/kg daily); or B) SEQ ID NO: 46 3 mg/kg 2×/week. A second experiment was conducted and mice dosed with SEQ ID NO: 70 at 10 mg/kg 2× week or SEQ ID NO: 87 at 30 mg/kg 2× week. From day 3 to day 21, body weights and clinical scores (tail limping and/or hind limb paralysis) are taken daily. All mice were daily monitored for signs of EAE, and the observations were scored as follows: 0=no disease, 1=tail paralysis, 2=hind limb weakness, 3=hind limb paralysis, 4=hind limb plus forelimb paralysis, and 5=moribund. See FIGS. 6A and 6B. SEQ ID NO: 87, 89 and 91 were also tested in the PLP-EAE murine model, with SEQ ID NO: 91 showing similar improved clinical score as SEQ ID NO: 87. See FIG. 7.

Upon sacrifice the spinal cords of the mice were removed and saved for subsequent histologic assessment.

The bone morphogenetic protein (BMP) and growth and differentiation factors (GDF) ligands play a role in neurodegeneration and possibly autoimmunity. Blockage of (BMP) signaling is has been reported to stimulate neurogenesis and oligodendrocytes from neural stem cells (NSCs). However, sera of untreated relapsing-remitting multiple sclerosis (RR-MS) patients express significantly higher levels of BMP-2 compared to sera of healthy control and it has been suggest that BMP-2 sera levels may play a role in the failure of remyelination and neuro-regeneration in RR-MS. In this experiment, we demonstrate that using a BMP antagonist (e.g., ALK-6 polypeptide of this disclosure) leads to a reduction in disease severity. The soluble ligand trap ALK6-Fc (SEQ ID NO: 70 and SEQ ID NO: 87) potently inhibit the development of clinical symptoms in the PLP-EAE murine model of multiple sclerosis, however this data shows that post day 25, SEQ ID NO: 70 was not as potent as compared to SEQ ID NO: 87. See FIG. 6B. The soluble ligand trap ALK6-Fc (SEQ ID NO: 87 and SEQ ID NO: 91) potently inhibit the development of clinical symptoms in the PLP-EAE murine model of multiple sclerosis. See FIG. 7.

In this experiment, we show loss of BMP10 binding by an ALK6 fusion protein of this disclosure fails to significantly to decrease the clinical symptoms in the PLP-EAE murine model of multiple sclerosis. Provided herein is a method for treating multiple sclerosis comprising administering an effective amount of the soluble ALK6 Fc fusion polypeptide of this disclosure, and that binds BMP10, to a subject in need thereof.

### Example 6: EAE Induction by Active Immunization in C57BL/6 Mice and Treatment with ALK-6 Fc Fusion Proteins

EAE is induced in C57BL/6 mice (female 9 to 13 weeks old at immunization) by immunization with an emulsion of Myelin Oligodendrocyte Glycoprotein (MOG₃₅₋₅₅ or MOG₁₋₁₂₅) in complete Freund's adjuvant (CFA), followed by administration of pertussis toxin (PTX) in PBS, first on the day of immunization and then again the following day. This method provides a model of chronic Multiple Sclerosis. The emulsion provides antigen which initiates expansion and differentiation of MOG-specific autoimmune T cells and the PTX enhances EAE development by providing additional adjuvant and facilitating entrance of autoimmune T cells into the CNS. After immunization, clinical signs and changes in body weight are observed one or two weeks later. Bio Protoc. 2019 Dec. 20; 9 (24): e3453.

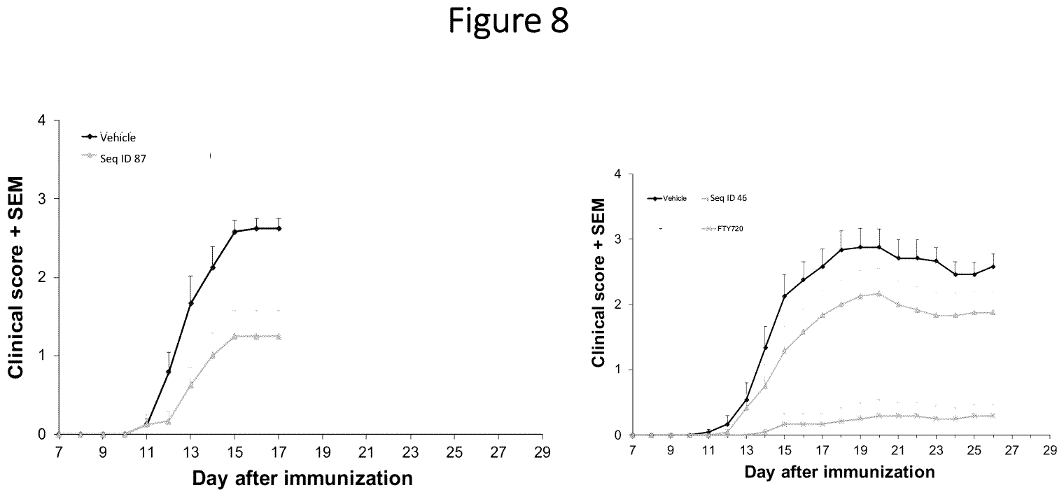

Following EAE induction, mice were randomly divided into 2 groups and starting on Day 7 mice are dosed with A) SEQ ID NO: 87 (20 mg/kg); or B) SEQ ID NO: 46 (10 mg/kg). From day 3 to day 26, body weights and clinical scores (tail limping and/or hind limb paralysis) are taken daily. All mice were monitored daily for signs of EAE, and the observations were scored as follows: 0=no disease, 1=tail paralysis, 2=hind limb weakness, 3=hind limb paralysis, 4=hind limb plus forelimb paralysis, and 5=moribund. See FIG. 8. The soluble ligand trap ALK6-Fc (SEQ ID NO: 87) also potently inhibits the development of clinical symptoms in the MOG EAE murine model of multiple sclerosis. See FIG. 8.

Blood will be collected and about 25 ul of blood serum will be analyzed for Neurofilament light chain content (NfL).

Neurofilament light chain (NfL) is a neuronal cytoplasmic protein highly expressed in large caliber myelinated axons. Its levels increase in cerebrospinal fluid (CSF) and blood proportionally to the degree of axonal damage in a variety of neurological disorders, including inflammatory, neurodegenerative, traumatic and cerebrovascular diseases. NfL is a subunit of neurofilaments (Nfs), which are cylindrical proteins exclusively located in the neuronal cytoplasm. Nfs confer structural stability to neurons and are present in dendrites and neuronal soma, as well as in axons, where their expression is particularly high. Since Nfs enable the radial growth of axons, larger myelinated axons abundantly express Nfs and NfL. In response to CNS axonal damage because of inflammatory, neurodegenerative, traumatic or vascular injury, the release of NfL sharply increases.

In embodiments provided herein, measurement of NfL is used to monitor the treatment of a chronic MS patient with an ALK6 Fc fusion polypeptide of this disclosure. In certain embodiments provided herein, measurement of NfL is used to show slowing, prevention and/or improvement of chronic MS in a patient treated with an ALK6 Fc fusion polypeptide of this disclosure. In embodiments, NfL is measured using a single molecule array (e.g. Simoa), which allows subpicogram levels of protein to be detected in the serum sNfL or cerebrospinal fluid cNfL. In other embodiments, NfL is measured using an ELISA or electrochemiluminescence assay.

### Example 7: Induction of the Cuprizone Model and Treatment with ALK-6 Fe Fusion Proteins

Bis-cyclohexanone-oxaldihydrazone, colloquially known as cuprizone, is a copper chelator that, when fed to rodents, results in a loss of oligodendrocytes and thus, myelin, in specific brain regions, with limited BBB disturbance and infiltration of peripheral immune cells. In particular, dietary consumption of cuprizone causes demyelination of specific brain white matter regions, such as corpus callosum and hippocampus. Cuprizone is widely used to generate toxin provoked demyelination model for study of demyelination and remyelination in MS as well as testing of potential pro-myelinating treatments.

The histopathological hallmark of the cuprizone model is oligodendrocyte death and subsequent demyelination in the corpus callosum, superior cerebellar peduncles, hippocampus and several other areas of the mouse brain. In this model, myelin destabilization and loss begin at 2-3 weeks of cuprizone administration and peaks at week 4-5, myelin levels stay low for weeks. This progressive demyelination is accompanied by robust astrogliosis and microgliosis. Astrogliosis begins coincident with demyelination after 2 weeks on cuprizone diet. Astrocyte densities peak around 5-6 weeks and stay high for weeks. Microglia also proliferate and expand after 2 weeks, and their density peaks at 5-6 weeks. Demyelination of brain white matter coincides with behavioral changes and loss of cognitive and motor function. While the cuprizone diet causes widespread demyelination, removal of cuprizone permits remyelination of the corpus callosum and cerebellar peduncles after a seven-week cuprizone diet or even a longer seven-month diet, although remyelination is less efficient with chronic cuprizone consumption.

Cuprizone toxicity is one of the few models of MS that produces grey matter demyelination, specifically in the cortex, hippocampus, and deep grey matter nuclei. Cortical grey matter demyelination is evident after 4 weeks of cuprizone treatment, but microglia/macrophage density in this region remains near baseline levels. Likewise, cuprizone administration results in layer-specific cortical degeneration, with demyelination enriched within layers five and six. Given that cuprizone administration induces both white and grey matter demyelination, it remains a highly relevant and useful model for studying the biological underpinnings of MS lesions.

In this experiment, four groups of 7-8-week-old C57BL/6 male mice (Jackson Laboratories) will be fed with 0.2% cuprizone (w/w: bis-cyclohexanone-oxaldihydrazone, (Envigo, Indianapolis IN) for 5 weeks (day 35, maximum demyelination) to induce acute demyelination. One group will be treated with a soluble ALK6 Fc fusion polypeptide of this disclosure (e.g., SEQ ID NO: 46) from day zero while the other group will be treated with saline.

After 5 weeks of treatment the two groups of mice will be sacrificed and histologic evaluation of brain sections by Luxol fast blue staining and immunohistochemistry using antibodies for MBP (myelin). Two groups will receive a cuprizone diet for 5 weeks and then transferred to a regular diet for 10 days. One group will be treated with saline for the 10 days while the other group will receive a soluble ALK6 Fc fusion polypeptide of this disclosure (e.g., SEQ ID NO: 46) will be administered via subcutaneous (SC) injection (e.g., 10 mg/kg/mouse) daily for 10 days. A separate control group on a normal diet (no Cuprizone) with no vehicle dosing will be used as a further comparator. Mice will be sacrificed at the end of 4 week test period and histology evaluated on brain sections by immunohistochemistry using antibodies for MBP (myelin); PDGFR-alpha (progenitor oligos); ASPA (mature oligos).

In this experiment, we demonstrate that using a ALK-6 Fc fusion polypeptide of this disclosure (e.g., BMP antagonist) leads to a reduction in disease severity. In certain embodiments, we demonstrate that using a ALK-6 Fc fusion polypeptide of this disclosure (e.g., BMP antagonist) leads to remyelination. NfL, in the serum sNfL or cerebrospinal fluid cNfL will also be measured to monitor effects on neuron survival.

### Example 8: SOD1^{G934} Mice Model of ALS and Treatment with ALK-6 Fc Fusion Proteins

SOD1-G93A mice express human SOD1 with the G93A mutation under control of the cistronic human SOD1 promotor. Mutations in this gene have been linked to familial amyotrophic lateral sclerosis (ALS or Lou Gehrig's disease). The SOD1-G93A mice show a phenotype, similar to ALS in humans, including loss of cutaneous small fibers. Mice develop paralysis in one or more limbs within a few weeks of age. This model presents a very severe phenotype including motor deficits, SOD1 aggregations, loss of spinal motor neurons, and a mean survival rate of only 18.5 weeks. Additionally, animals present dysphagia and associated tongue atrophy that are congruent with the hypoglossal nucleus pathology in patients with ALS. These mice are a valuable tool to study the influence of new drugs on neuromuscular disorders such as ALS.

In this experiment, approximately 6-week old female transgenic mice (B6SJL-Tg(SOD-1*G93A)1Gur/J) are stored 3-4 mice per cage with controlled 12 h light/dark cycle and normal temperature and relative humidity ranges. After a 3-7 day acclimation, all sixteen (16) mice will be grouped by body weight into 2 groups: vehicle and a ALK-6 Fc fusion peptide (e.g., SEQ ID NO: 46) and will be dosed subcutaneously twice weekly starting at 8 weeks of age for 12 weeks, body weights and survival are recorded. Retro-orbital blood will be collected at 10, 15, and 20 weeks of age and about 25 ul of blood serum and 20 μL of cNfL will be analyzed for Neurofilament light chain content (NfL), which is a reliable biological marker to monitor neuronal death and predict ALS progression, disease improvement and survival.

The role of NfL as a biomarker has been largely reported in multiple sclerosis (MS), Alzheimer's disease (AD), frontotemporal dementia (FTD), ALS, atypical parkinsonian disorders (APD) and traumatic brain injury (TBI). NfL is a sensitive but unspecific marker of axonal injury wherein CSF and blood NfL is higher in patients with ALS compared with healthy and neurological controls. Recently, a serum NfL cut-off value of 62 μg/mL was found to have a sensitivity of 85.5% (95% CI 78% to 91.2%) and a specificity of 81.8% (95% CI 74.9% to 87.4%) in distinguishing ALS from other neurological disorders.

In embodiments provided herein, measurement of NfL is used to monitor the treatment of an ALS patient with an ALK6 Fc fusion polypeptide of this disclosure. In certain embodiments provided herein, measurement of NfL is used to show slowing, prevention and/or improvement of ALS in a patient treated with an ALK6 Fc fusion polypeptide of this disclosure. In embodiments, NfL is measured using a single molecule array (e.g. Simoa), which allows subpicogram levels of protein to be detected (sNfL). In other embodiments, NfL is measured using an ELISA or electrochemiluminescence assay.

### Example 9: 5×FAD Alzheimer Animal Model and Treatment with ALK-6 Fe Fusion Proteins

5×FAD mice express human APP and PSEN1 transgenes with a total of five AD-linked mutations: the Swedish (K670N/M671L). Florida (I716V), and London (V717I) mutations in APP, and the M146L and L286V mutations in PSEN1. These widely used mice recapitulate many AD-related phenotypes and have a relatively early and aggressive presentation. Amyloid plaques, accompanied by gliosis, are seen in mice as young as two months of age. Amyloid pathology is more severe in females than in males. Neuron loss occurs in multiple brain regions, beginning at about 6 months in the areas with the most pronounced amyloidosis NIL levels are noted to increase at the time of noted neuronal loss. Mice display a range of cognitive and motor deficits.

The 5×FAD model rapidly develops severe amyloid pathology. These mice accumulate high levels of intraneuronal Δ042, beginning around 1.5 months of age. Extracellular amyloid deposition begins around 2 months, first in the subiculum and layer V of the cortex, and increasing rapidly with age. Plaques are found throughout the hippocampus and cortex by six months; in older mice, plaques are present in the thalamus, brainstem, and olfactory bulb, but are absent from the cerebellum. Astrogliosis and microgliosis begin around two months, developing in parallel with plaque deposition.

Synaptic degeneration, assessed as whole-brain levels of the presynaptic marker synaptophysin, begins by four months of age. Neuron loss has been observed in multiple brain regions in this model. In the areas with the most severe amyloidosis—the subiculum and cortical layer V—neuron loss and myelin abnormalities begin at about 6 months of age. sNfL and CNfL are increased over a 12 month period reflecting neuronal death.

In this experiment, 5×FAD mice on a congenic C57BL/6J background (Jackson Laboratories) are treated (10 mice per group) with vehicle (e.g., saline) or an ALK-6 Fc fusion peptide (e.g., SEQ ID NO: 46) for three months beginning at 2.5 months of age. Serum samples from retro-orbital bleeds are taken at day 0 and then monthly for 4 months and evaluated for serum sNfL and cerebrospinal fluid cNfL NfL levels are determined using a simoa single molecule array system (Quanterix) as well as a Luminex inflammatory cytokine panel. At the conclusion of the experiment spinal cord fluid is removed and NfL levels are determined. To assess effect on neuronal degeneration, brains are removed and stained using traditional histochemical analysis and immunohistochemical analysis for oligodendrocytes, Astrocytes, GFAP+ and myelin.

In embodiments provided herein are methods for treating, preventing, slowing and/or improving Alzheimer's disease (AD) in a patient in need thereof using an ALK6 Fc fusion polypeptide of this disclosure. In certain embodiments provided herein, measurement of NfL is used to monitor the treatment of an AD patient with an ALK6 Fc fusion polypeptide of this disclosure. In certain embodiments provided herein, measurement of NfL is used to show slowing, prevention and/or improvement of AD in a patient treated with an ALK6 Fc fusion polypeptide of this disclosure. In embodiments, NfL is measured using a single molecule array (e.g. Simoa), which allows subpicogram levels of protein to be detected (NfL). In other embodiments, NfL is measured using an ELISA or electrochemiluminescence assay.

### Example 10: Induction of DTH in C57BL/6J Mice and Treatment with ALK-6 Fe Fusion Polypeptide—ALK6-Fc does not Affect the Adaptive Immune System

Delayed-type hypersensitivity (DTH), also known as a type IV hypersensitivity reaction, is an in vivo assay of cell-mediated immune function and a useful approach for evaluating cell-mediated immune responses associated with Th1 and Th17 reactivity. DTH reactions are divided into two phases: the sensitization phase, referring to the initial immunization with specific antigen, and the efferent or challenge phase of the DTH response, which usually follows 6 to 14 days after sensitization. DTH reactions are mediated by soluble or cell-associated antigens primarily involving CD4+ or CD8+ T cell activation. These reactions are characterized by the release of mediators from activated T cells. The T cells then activate local endothelial cells and recruit macrophages, which results in local inflammation and swelling. Thus, DTH murine models are used herein to study activation of the adaptive immune system using the ALK-6 Fc fusion polypeptides of this disclosure.

In this experiment mice were treated with methylated bovine serum albumin in Complete Freund's Adjuvant (mBSA/CFA) wherein the emulsion was injected at 2 sites in the back, 50 L/site, intradermally (i.d.). On day 0 and day 3, group 1 mice received saline while group 2 received an ALK-6 Fc fusion polypeptide of this disclosure (SEQ ID NO: 46 and SEQ ID NO: 87), 10 mg/kg and 30 mg/kg respectively. Additional groups of mice received either dexamethasone (1 mg/kg) or Gileya® (FTY720), both of which are known immunosuppressive therapeutics of the adaptive immune response and in this experiment effectively reduced and/or suppressed the DTH reaction. The no DTH group was the corresponding control not injected with mBSA/CFA.

On day 5 each mouse had one hind paw injected subcutaneously with either PBS (Group 1) or mBSA solution in PBS, 20 μL/paw. The opposite hind paw did not receive any injection.

Readouts included body weight, paw thickness, and paw weight. Body weight was measured 3×/week (Monday, Wednesday, Friday; weighing may be delayed 1 day if these fall on a major US holiday), starting on Day 0. Paw thickness On Day 5, before the challenge administration, baseline hind paw thickness of all mice (1 paw/mouse) was measured. On Day 6, injected-paw thickness was measured again, 20 to 24 hours after challenge injection. Thickness measurements will be performed blind, by a person unaware of the group or previous measurements. Paw weight After paw thickness was measured on Day 6, all mice were sacrificed and injected paws collected (from the hairline), weighed, and then cut in half to allow for MPO and cytokine analysis. See FIGS. 9 and 10.

In this experiment, we show that using a BMP antagonist (e.g., SEQ ID NO: 87 and SEQ ID NO: 46) does not suppress an adaptive immune response. In other words, the ALK6 Fc fusion proteins of this disclosure are not immunosuppressive therapeutics of the adaptive immune system. Provided herein is a method for treating multiple sclerosis comprising administering an effective amount of the soluble ALK6 Fc fusion polypeptide of this disclosure to a subject in need thereof, wherein the efficacy of the ALK6 Fc fusion polypeptide is likely not due a suppression of the adaptive immune response similar to other approved therapies.

1. A soluble recombinant bone morphogenetic protein receptor type-1B fusion protein comprising an ALK6 extra cellular domain (ECD) polypeptide and a Fc sequence, wherein the ALK6 ECD polypeptide comprises an amino acid sequence that is at least 95%, 96%, 97%, 98%, 99%, or 100% identical to an ALK6 ECD sequence selected from SEQ ID NO: 4; SEQ ID NO: 69, and SEQ ID NO: 113.

2. The fusion protein of claim 1, wherein amino acid position 60 is a glycine residue.

3. The fusion protein of claim 1, wherein the polypeptide binds BMP10.

4-15. (canceled)

16. A soluble recombinant bone morphogenetic protein receptor type-1B fusion protein comprising an ALK6 ECD polypeptide and a Fc sequence, wherein the ALK6 ECD polypeptide comprises an amino acid sequence selected from a sequence of SEQ ID NO: 81:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₅₅-Xaa₈₆-Xaa₈₇-Xaa₅₅-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀-Lys₁₀₁-Asn₁₀₂-Arg₁₀₃-Asp₁₀₄-Phe₁₀₅-Val₁₀₆-Xaa₁₀₇-Xaa₁₀₈-Xaa₁₀₉-Xaa₁₁₀-Xaa₁₁₁-Xaa₁₁₂-Xaa₁₁₃;
wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₇ is absent;
Xaa₁₀₈ is absent;
Xaa₁₀₉ is absent;
Xaa₁₁₀ is absent;
Xaa₁₁₁ is absent;
Xaa₁₁₂ is absent; and,
Xaa₁₁₃ is absent.

17. The soluble protein of claim 16, wherein the ALK6 ECD polypeptide comprises an amino acid sequence that is at least 95%, 96%, 97, 98%, 99%, or 100% identical to an ALK6 ECD sequence selected from SEQ ID NO: 4, 13, 69, 86, 92, 94, 96, 98, and 100.

18. The fusion protein of claim 16, wherein the ALK6 ECD polypeptide comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52T, V53T, T55A, L59M, L61Y, R69K, T71S, I73K, P74A, H75Q, Q76L, S79T, T84R, E85T, E85_, R86N, N87L, E88_, K91Q, D92L, H94Q, D104A, or a combination thereof, wherein _ is a deletion at that position.

19-30. (canceled)

31. A soluble recombinant bone morphogenetic protein receptor type-1B fusion protein comprising an ALK6 ECD polypeptide and a Fc sequence, wherein the ALK6 ECD polypeptide comprises an amino acid sequence selected from a sequence of SEQ ID NO: 108:
Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Val₁₆-Leu₁₇-Arg₁₈-Cys₁₉-Xaa₂₀-Cys₂₁-Xaa₂₂-Xaa′₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₂-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₅₅-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀-Lys₁₀₁-Asn₁₀₂-Arg₁₀₃-Asp₁₀₄-Phe₁₀₅-Val₁₀₆-Xaa₁₀₇-Xaa₁₀₈-Xaa₁₀₉-Xaa₁₁₀-Xaa₁₁₁-Xaa₁₁₂-Xaa₁₁₃;
wherein:
Xaa₁₅ is K or P;
Xaa₂₀ is V, F, K or Y;
Xaa₂₂ is G, H or S;
Xaa′₂₂ is absent or A;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₇ is absent;
Xaa₁₀₈ is absent;
Xaa₁₀₉ is absent;
Xaa₁₁₀ is absent;
Xaa₁₁₁ is absent;
Xaa₁₁₂ is absent; and,
Xaa₁₁₃ is absent.

32. The fusion protein of claim 31, wherein the ALK6 ECD polypeptide comprises an amino acid sequence that is at least 95%, 96%, 97, 98%, 99%, or 100% identical to an ALK6 ECD sequence selected from SEQ ID NO: 13, 86, 90, 92, 94, 96, 98, 100, 102, 104, and 110.

33. The fusion protein of claim 31, wherein the ALK6 ECD polypeptide comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52T, V53T, T55A, L59M, L61Y, R69K, T71S, I73K, P74A, H75Q, Q76L, S79T, T84R, E85T, E85_, R86N, N87L, E88_, K91Q, D92L, H94Q, D104A, or a combination thereof, wherein _ is a deletion at that position.

34-40. (canceled)

41. The fusion protein of claim 1, further comprising a linker sequence.

42. The fusion protein of claim 1, further comprising a linker sequence selected from SEQ ID NO: 6, SEQ ID NO: 7 or SEQ ID NO: 8.

43. The fusion protein of claim 1, wherein the Fc sequence is according to SEQ ID NO: 10, SEQ ID NO: 106 or SEQ ID NO: 107.

44-50. (canceled)

51. The soluble recombinant bone morphogenetic protein receptor type-1B fusion protein according to claim 1 comprising: an ALK6 extra cellular domain (ECD), a linker sequence and an Fc domain sequence wherein the ALK6 ECD sequence comprises an amino acid sequence that is at least 95%, 96%, 97%, 98%, 99%, or 100% identical to an ALK6 ECD sequence selected from SEQ ID NO: 13 69, 86, 90, 92, 94, 96, 98, 100, 102, 104, 110 and 113 to 129, wherein amino acid position 60 is a glycine residue.

52. (canceled)

53. (canceled)

54. The fusion protein of claim 51, wherein the ALK6 ECD comprises one or more point mutations selected from K20Y, K20F, K20V, H22S, H22G, H22E, H23S, H23L, H23G, H24S.

55. The fusion protein of claim 54, wherein the ALK6 ECD comprises an amino acid insertion between position 22 and position 23 of an alanine (A) residue or a glycine (G) residue.

56-60. (canceled)

61. A pharmaceutical composition comprising a fusion protein according to claim 1; and, at least one pharmaceutical acceptable carrier or buffer.

62-90. (canceled)

91. A method of treating multiple sclerosis, comprising: administering an effective amount of a pharmaceutical composition to a subject in need thereof, wherein the composition comprises a soluble recombinant bone morphogenetic protein receptor type-1B fusion protein according to claim 1 wherein amino acid position 60 is a glycine (G) residue and the fusion protein binds BMP10.

92. The method of claim 91, wherein the ALK6 ECD sequence comprises at least one point mutation as compared to SEQ ID NO: 69.

93. The method of claim 92, wherein the point mutation is selected from K20Y, K20F, K20V, H22S, H22G, H22E, H23S, H23L, H23G, H24S.

94-96. (canceled)

97. The method of claim 93, wherein the ALK6 ECD comprises an amino acid insertion between position 22 and position 23 of an alanine (A) residue or glycine (G) residue.

98. (canceled)


SEQUENCE LISTING (113 sequences)

SEQ ID NO: 1 - protein, 113 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHR

SEQ ID NO: 2 - protein, 12 aa, synthetic construct
VVIGPFFDGSIR

SEQ ID NO: 3 - protein, 113 aa, synthetic construct
  VARIANT 15: note = K or P
  VARIANT 20: note = V, F, K or Y
  VARIANT 22: note = G, H, or S
  VARIANT 23: note = S, H, or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = T, E, or absent
  VARIANT 86: note = R or N
  VARIANT 87: note = N or L
  VARIANT 88: note = E, L, or absent
  VARIANT 91: note = K or Q
  VARIANT 92: note = D or L
  VARIANT 94: note = H or Q
  VARIANT 100: note = V, L, or absent
  VARIANT 101: note = V, K, or absent
  VARIANT 102: note = I, N, or absent
  VARIANT 103: note = G, R, or absent
  VARIANT 104: note = P, D, A, or absent
  VARIANT 105: note = May be absent
  VARIANT 106: note = F, V, or absent
  VARIANT 107: note = May be absent
  VARIANT 108: note = May be absent
  VARIANT 109: note = S, P, or absent
  VARIANT 110: note = May be absent
  VARIANT 111: note = R, H, or absent
  VARIANT 112: note = May be absent
  VARIANT 113: note = May be absent
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPXXXXXFXDGXIXHR

SEQ ID NO: 4 - protein, 100 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPL

SEQ ID NO: 5 - protein, 100 aa, synthetic construct
  VARIANT 15: note = K or P
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = T, E, or absent
  VARIANT 86: note = R or N
  VARIANT 87: note = N or L
  VARIANT 88: note = E, L, or absent
  VARIANT 91: note = K or Q
  VARIANT 92: note = D or L
  VARIANT 94: note = H or Q
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPL

SEQ ID NO: 6 - protein, 21 aa, synthetic construct
TGGGGSGGGGSGGGGSGGGGS

SEQ ID NO: 7 - protein, 4 aa, synthetic construct
TGGG

SEQ ID NO: 8 - protein, 20 aa, synthetic construct
EAAAKEAAAKEAAAKEAAAK

SEQ ID NO: 9 - protein, 19 aa, synthetic construct
MGWSCIILFLVATATGVHS

SEQ ID NO: 10 - protein, 225 aa, synthetic construct
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 12 - protein, 113 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRAFVDGPIHHR

SEQ ID NO: 13 - protein, 106 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRAFV

SEQ ID NO: 14 - protein, 112 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRAFVGPIHHR

SEQ ID NO: 15 - protein, 111 aa, synthetic construct
KKEDGESTAPTPRPKVLRCKCHGHCPEDSVNNICSTNGYCFTIIEEDDSGLPVVTSGCMGYEGSDFQCRDTPKAHLRRSIECCTERNECNKDLHPTLPPVVIGPFFDGSIR

SEQ ID NO: 16 - protein, 110 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTRNECNKDLHPTLPPVVIGPFFDGSIR

SEQ ID NO: 17 - protein, 111 aa, synthetic construct
KKEDGESTAPTPRPPVLRCKCHHHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 18 - protein, 111 aa, synthetic construct
KKEDGESTAPTPRPKVLRCYCHHHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 19 - protein, 111 aa, synthetic construct
KKEDGESTAPTPRPKVLRCKCSHHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 20 - protein, 111 aa, synthetic construct
KKEDGESTAPTPRPKVLRCKCHGHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 21 - protein, 111 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 22 - protein, 111 aa, synthetic construct
KKEDGESTAPTPRPKVLRCKCHHHCPDDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 23 - protein, 111 aa, synthetic construct
KKEDGESTAPTPRPKVLRCKCHHHCPEDSINNICSTDGYCFTMIEEDDSGLPVVTSGCLGLEGSDFQCRDTPIPHQRRSIECCTERNECNKDLHPTLPPVVIGPFFDGSIR

SEQ ID NO: 24 - protein, 111 aa, synthetic construct
KKEDGESTAPTPRPKVLRCKCHHHCPEDSVNNTCSTDGYCFTMIEEDDSGLPVVTSGCLGLEGSDFQCRDTPIPHQRRSIECCTERNECNKDLHPTLPPVVIGPFFDGSIR

SEQ ID NO: 25 - protein, 111 aa, synthetic construct
KKEDGESTAPTPRPKVLRCKCHHHCPEDSVNNICITDGYCFTMIEEDDSGLPVVTSGCLGLEGSDFQCRDTPIPHQRRSIECCTERNECNKDLHPTLPPVVIGPFFDGSIR

SEQ ID NO: 26 - protein, 111 aa, synthetic construct
KKEDGESTAPTPRPKVLRCKCHHHCPEDSVNNICSTNGYCFTMIEEDDSGLPVVTSGCLGLEGSDFQCRDTPIPHQRRSIECCTERNECNKDLHPTLPPVVIGPFFDGSIR

SEQ ID NO: 27 - protein, 111 aa, synthetic construct
KKEDGESTAPTPRPKVLRCKCHHHCPEDSVNNICSTDGHCFTMIEEDDSGLPVVTSGCLGLEGSDFQCRDTPIPHQRRSIECCTERNECNKDLHPTLPPVVIGPFFDGSIR

SEQ ID NO: 28 - protein, 111 aa, synthetic construct
KKEDGESTAPTPRPKVLRCKCHHHCPEDSVNNICSTDGYCFAIIEEDDSGLPVVTSGCLGLEGSDFQCRDTPIPHQRRSIECCTERNECNKDLHPTLPPVVIGPFFDGSIR

SEQ ID NO: 29 - protein, 111 aa, synthetic construct
KKEDGESTAPTPRPKVLRCKCHHHCPEDSVNNICSTDGYCFAMIEEDDSGLPVVTSGCLGLEGSDFQCRDTPIPHQRRSIECCTERNECNKDLHPTLPPVVIGPFFDGSIR

SEQ ID NO: 30 - protein, 111 aa, synthetic construct
KKEDGESTAPTPRPKVLRCKCHHHCPEDSVNNICSTDGYCFTIIEEDDSGLPVVTSGCLGLEGSDFQCRDTPIPHQRRSIECCTERNECNKDLHPTLPPVVIGPFFDGSIR

SEQ ID NO: 31 - protein, 111 aa, synthetic construct
KKEDGESTAPTPRPKVLRCKCHHHCPEDSVNNICSTDGYCFTMIEEDDQGLPVVTSGCLGLEGSDFQCRDTPIPHQRRSIECCTERNECNKDLHPTLPPVVIGPFFDGSIR

SEQ ID NO: 32 - protein, 111 aa, synthetic construct
KKEDGESTAPTPRPKVLRCKCHHHCPEDSVNNICSTDGYCFTMIEEDDQGETTLASGCLGLEGSDFQCRDTPIPHQRRSIECCTERNECNKDLHPTLPPVVIGPFFDGSIR

SEQ ID NO: 33 - protein, 111 aa, synthetic construct
KKEDGESTAPTPRPKVLRCKCHHHCPEDSVNNICSTDGYCFTMIEEDDSGLPVVTSGCMKYEGSDFQCRDTPIPHQRRSIECCTERNECNKDLHPTLPPVVIGPFFDGSIR

SEQ ID NO: 34 - protein, 111 aa, synthetic construct
KKEDGESTAPTPRPKVLRCKCHHHCPEDSVNNICSTDGYCFTMIEEDDSGLPVVTSGCMGLEGSDFQCRDTPIPHQRRSIECCTERNECNKDLHPTLPPVVIGPFFDGSIR

SEQ ID NO: 35 - protein, 111 aa, synthetic construct
KKEDGESTAPTPRPKVLRCKCHHHCPEDSVNNICSTDGYCFTMIEEDDSGLPVVTSGCLKLEGSDFQCRDTPIPHQRRSIECCTERNECNKDLHPTLPPVVIGPFFDGSIR

SEQ ID NO: 36 - protein, 111 aa, synthetic construct
KKEDGESTAPTPRPKVLRCKCHHHCPEDSVNNICSTDGYCFTMIEEDDSGLPVVTSGCLGYEGSDFQCRDTPIPHQRRSIECCTERNECNKDLHPTLPPVVIGPFFDGSIR

SEQ ID NO: 37 - protein, 111 aa, synthetic construct
KKEDGESTAPTPRPKVLRCKCHHHCPEDSVNNICSTDGYCFTMIEEDDSGLPVVTSGCLGLEGSDFQCRDTPKAQLRRSIECCTERNECNKDLHPTLPPVVIGPFFDGSIR

SEQ ID NO: 38 - protein, 111 aa, synthetic construct
KKEDGESTAPTPRPKVLRCKCHHHCPEDSVNNICSTDGYCFTMIEEDDSGLPVVTSGCLGLEGSDFQCRDTPKAHQRRSIECCTERNECNKDLHPTLPPVVIGPFFDGSIR

SEQ ID NO: 39 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPQLRRSIECCTERNECNKDLHPTLPPVVIGPFFDGSIR

SEQ ID NO: 40 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCRERNLCNKDLHPTLPPVVIGPFFDGSIR

SEQ ID NO: 41 - protein, 111 aa, synthetic construct
KKEDGESTAPTPRPKVLRCKCHHHCPEDSVNNICSTDGYCFTMIEEDDSGLPVVTSGCLKLEGSDFQCRDTPKAQLRRSIECCTERNECNKDLHPTLPPVVIGPFFDGSIR

SEQ ID NO: 42 - protein, 110 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCRTNLCNQYLQPTLPPVVIGPFFDGSIR

SEQ ID NO: 43 - protein, 110 aa, synthetic construct; identical to SEQ ID NO: 16

SEQ ID NO: 44 - protein, 111 aa, synthetic construct
KKEDGESTAPTPRPKVLRCKCSGHCPDDAVNNTCITNGYCFAIIEEDDSGLPVVASGCMKYEGSDFQCKDSPKAQLRRTIECCTERNECNKDLHPTLPPVVIGPFFDGSIR

SEQ ID NO: 45 - protein, 111 aa, synthetic construct
KKEDGESTAPTPRPKVLRCKCHGHCPEDSVNNICSTNGYCFTIIEEDDSGLPVVTSGCMKYEGSDFQCRDTPKAHLRRSIECCTERNECNKDLHPTLPPVVIGPFFDGSIR

SEQ ID NO: 46 - protein, 357 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 359 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRAFVDGPIHHRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 352 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRAFVTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 378 aa, synthetic construct
MGWSCIILFLVATATGVHSKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 365 aa, synthetic construct
MGWSCIILFLVATATGVHSKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 14 aa, synthetic construct
LKNRDFVDGPIHHR

SEQ ID NO: 52 - protein, 357 aa, synthetic construct
KKEDGESTAPTPRPKVLRCKCHGHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 357 aa, synthetic construct
KKEDGESTAPTPRPKVLRCKCHHHCPEDSVNNICSTNGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 357 aa, synthetic construct
KKEDGESTAPTPRPKVLRCKCHHHCPEDSVNNICSTDGYCFAIIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 357 aa, synthetic construct
KKEDGESTAPTPRPKVLRCKCHHHCPEDSVNNICSTDGYCFTMIEEDDSGLPVVTSGCMKYEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 357 aa, synthetic construct
KKEDGESTAPTPRPKVLRCKCHHHCPEDSVNNICSTDGYCFTMIEEDDSGLPVVTSGCLKLEGSDFQCRDTPKAQLRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 111 aa, synthetic construct; identical to SEQ ID NO: 19

SEQ ID NO: 58 - protein, 357 aa, synthetic construct
KKEDGESTAPTPRPKVLRCKCSHHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 111 aa, synthetic construct
KKEDGESTAPTPRPKVLRCKCSGHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 60 - protein, 357 aa, synthetic construct
KKEDGESTAPTPRPKVLRCKCSGHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 111 aa, synthetic construct; identical to SEQ ID NO: 21

SEQ ID NO: 62 - protein, 357 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 111 aa, synthetic construct
KKEDGESTAPTPRPKVLRCYCHGHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 64 - protein, 357 aa, synthetic construct
KKEDGESTAPTPRPKVLRCYCHGHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 111 aa, synthetic construct; identical to SEQ ID NO: 18

SEQ ID NO: 66 - protein, 357 aa, synthetic construct
KKEDGESTAPTPRPKVLRCYCHHHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 111 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFVAGPIR

SEQ ID NO: 68 - protein, 357 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFVAGPIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 106 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 70 - protein, 352 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 113 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVAGPIHHR

SEQ ID NO: 72 - protein, 359 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVAGPIHHRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVAGPIR

SEQ ID NO: 74 - protein, 357 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVAGPIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGSIR

SEQ ID NO: 76 - protein, 357 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFFDGPIR

SEQ ID NO: 78 - protein, 357 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFFDGPIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 111 aa, synthetic construct
  VARIANT 15: note = K or P
  VARIANT 20: note = V, F, K or Y
  VARIANT 22: note = G, H, or S
  VARIANT 23: note = S, H, or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = T, E, or absent
  VARIANT 86: note = R or N
  VARIANT 87: note = N or L
  VARIANT 88: note = E, L, or absent
  VARIANT 91: note = K or Q
  VARIANT 92: note = D or L
  VARIANT 94: note = H or Q
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPVVIGPFFDGSIR

SEQ ID NO: 80 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIR

SEQ ID NO: 81 - protein, 113 aa, synthetic construct
  VARIANT 15: note = K or P
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = T, E, or absent
  VARIANT 86: note = R or N
  VARIANT 87: note = N or L
  VARIANT 88: note = E, L, or absent
  VARIANT 91: note = K or Q
  VARIANT 92: note = D or L
  VARIANT 94: note = H or Q
  VARIANT 107: note = May be absent
  VARIANT 108: note = May be absent
  VARIANT 109: note = May be absent
  VARIANT 110: note = May be absent
  VARIANT 111: note = May be absent
  VARIANT 112: note = May be absent
  VARIANT 113: note = May be absent
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPLKNRDFVDGPIHHR

SEQ ID NO: 82 - protein, 113 aa, synthetic construct
  VARIANT 15: note = K or P
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = T, E, or absent
  VARIANT 86: note = R or N
  VARIANT 87: note = N or L
  VARIANT 88: note = E, L, or absent
  VARIANT 91: note = K or Q
  VARIANT 92: note = D or L
  VARIANT 94: note = H or Q
  VARIANT 107: note = D or A
  VARIANT 109: note = P or S
  VARIANT 111: note = May be absent
  VARIANT 112: note = May be absent
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPLKNRDFVXGXIHHR

SEQ ID NO: 83 - protein, 113 aa, synthetic construct
  VARIANT 15: note = K or P
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = T, E, or absent
  VARIANT 86: note = R or N
  VARIANT 87: note = N or L
  VARIANT 88: note = E, L, or absent
  VARIANT 91: note = K or Q
  VARIANT 92: note = D or L
  VARIANT 94: note = H or Q
  VARIANT 104: note = D or A
  VARIANT 107: note = May be absent
  VARIANT 109: note = P or S
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPLKNRXFVDGXIHHR

SEQ ID NO: 84 - protein, 356 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPVVIGPFFDGSIREAAAKEAAAKEAAAKEAAAKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 340 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPVVIGPFFDGSIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 106 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 87 - protein, 351 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 111 aa, synthetic construct
KKEDGESTAPTPRPKVLRCYCSGHCPEDSVNNICSTDGYCFTMIEEDDSGLPVVTSGCLKLEGSDFQCRDTPIPHQRRSIECCTERNECNKDLHPTLPPVVIGPFFDGSIR

SEQ ID NO: 89 - protein, 356 aa, synthetic construct
KKEDGESTAPTPRPKVLRCYCSGHCPEDSVNNICSTDGYCFTMIEEDDSGLPVVTSGCLKLEGSDFQCRDTPIPHQRRSIECCTERNECNKDLHPTLPPVVIGPFFDGSIRTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 107 aa, synthetic construct
KKEDGESTAPTPRPKVLRCYCSAGHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 91 - protein, 352 aa, synthetic construct
KKEDGESTAPTPRPKVLRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 106 aa, synthetic construct
KKEDGESTAPTPRPKVLRCYCSGHCPEDSVNNICSTNGYCFTMIEEDDSGLPVVTSGCLGLEGSDFQCRDTPIPHQRRSIECCTERNECNKDLHPTLPPLKNRDFV

SEQ ID NO: 93 - protein, 351 aa, synthetic construct
KKEDGESTAPTPRPKVLRCYCSGHCPEDSVNNICSTNGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 106 aa, synthetic construct
KKEDGESTAPTPRPKVLRCVCSGHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 95 - protein, 351 aa, synthetic construct
KKEDGESTAPTPRPKVLRCVCSGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 106 aa, synthetic construct
KKEDGESTAPTPRPKVLRCFCSGHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 97 - protein, 351 aa, synthetic construct
KKEDGESTAPTPRPKVLRCFCSGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 106 aa, synthetic construct
KKEDGESTAPTPRPKVLRCYCGSHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 99 - protein, 351 aa, synthetic construct
KKEDGESTAPTPRPKVLRCYCGSHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 106 aa, synthetic construct
KKEDGESTAPTPRPKVLRCFCGSHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 101 - protein, 351 aa, synthetic construct
KKEDGESTAPTPRPKVLRCFCGSHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 107 aa, synthetic construct
KKEDGESTAPTPRPKVLRCYCGASHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 103 - protein, 352 aa, synthetic construct
KKEDGESTAPTPRPKVLRCYCGASHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 107 aa, synthetic construct
KKEDGESTAPTPRPKVLRCYCSAGHCPEDSVNNICSTDGYCFTMIEEDDSGLPVVTSGCLKLEGSDFQCRDTPIPHQRRSIECCTERNECNKDLHPTLPPLKNRDFV

SEQ ID NO: 105 - protein, 352 aa, synthetic construct
KKEDGESTAPTPRPKVLRCYCSAGHCPEDSVNNICSTDGYCFTMIEEDDSGLPVVTSGCLKLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - protein, 224 aa, synthetic construct
THTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 225 aa, synthetic construct
THTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 114 aa, synthetic construct
  VARIANT 15: note = K or P
  VARIANT 20: note = V, F, K or Y
  VARIANT 22: note = G, H or S
  VARIANT 23: note = May be absent
  VARIANT 24: note = S, H or G
  VARIANT 28: note = E or D
  VARIANT 30: note = S or A
  VARIANT 31: note = V or I
  VARIANT 34: note = I or T
  VARIANT 36: note = S or I
  VARIANT 38: note = D or N
  VARIANT 40: note = Y or H
  VARIANT 43: note = T or A
  VARIANT 44: note = M or I
  VARIANT 50: note = S or Q
  VARIANT 52: note = L or E
  VARIANT 53: note = P or T
  VARIANT 54: note = V or T
  VARIANT 55: note = V or L
  VARIANT 56: note = T or A
  VARIANT 60: note = L or M
  VARIANT 61: note = G or K
  VARIANT 62: note = L or Y
  VARIANT 70: note = R or K
  VARIANT 72: note = T or S
  VARIANT 74: note = I or K
  VARIANT 75: note = P or A
  VARIANT 76: note = H or Q
  VARIANT 77: note = Q or L
  VARIANT 80: note = S or T
  VARIANT 85: note = T or R
  VARIANT 86: note = T, E, or absent
  VARIANT 87: note = R or N
  VARIANT 88: note = N or L
  VARIANT 89: note = E, L, or absent
  VARIANT 92: note = K or Q
  VARIANT 93: note = D or L
  VARIANT 95: note = H or Q
  VARIANT 108: note = May be absent
  VARIANT 109: note = May be absent
  VARIANT 110: note = May be absent
  VARIANT 111: note = May be absent
  VARIANT 112: note = May be absent
  VARIANT 113: note = May be absent
  VARIANT 114: note = May be absent
KKEDGESTAPTPRPXVLRCXCXAXHCPXDXXNNXCXTXGXCFXXIEEDDXGXXXXXSGCXXXEGSDFQCXDXPXXXXRRXIECCXXXXXCNXXLXPTLPPLKNRDFVDGPIHHR

SEQ ID NO: 109 - protein, 112 aa, synthetic construct
  VARIANT 15: note = K or P
  VARIANT 20: note = V, F, K or Y
  VARIANT 22: note = G, H or S
  VARIANT 23: note = May be absent
  VARIANT 24: note = S, H or G
  VARIANT 28: note = E or D
  VARIANT 30: note = S or A
  VARIANT 31: note = V or I
  VARIANT 34: note = I or T
  VARIANT 36: note = S or I
  VARIANT 38: note = D or N
  VARIANT 40: note = Y or H
  VARIANT 43: note = T or A
  VARIANT 44: note = M or I
  VARIANT 50: note = S or Q
  VARIANT 52: note = L or E
  VARIANT 53: note = P or T
  VARIANT 54: note = V or T
  VARIANT 55: note = V or L
  VARIANT 56: note = T or A
  VARIANT 60: note = L or M
  VARIANT 61: note = G or K
  VARIANT 62: note = L or Y
  VARIANT 70: note = R or K
  VARIANT 72: note = T or S
  VARIANT 74: note = I or K
  VARIANT 75: note = P or A
  VARIANT 76: note = H or Q
  VARIANT 77: note = Q or L
  VARIANT 80: note = S or T
  VARIANT 85: note = T or R
  VARIANT 86: note = T, E, or absent
  VARIANT 87: note = R or N
  VARIANT 88: note = N or L
  VARIANT 89: note = E, L, or absent
  VARIANT 92: note = K or Q
  VARIANT 93: note = D or L
  VARIANT 95: note = H or Q
KKEDGESTAPTPRPXVLRCXCXAXHCPXDXXNNXCXTXGXCFXXIEEDDXGXXXXXSGCXXXEGSDFQCXDXPXXXXRRXIECCXXXXXCNXXLXPTLPPVVIGPFFDGSIR

SEQ ID NO: 110 - protein, 107 aa, synthetic construct
KKEDGESTAPTPRPKVLRCYCSAGHCPEDSVNNICSTDGYCFTMIEEDDSGLPVVTSGCMKYEGSDFQCRDTPIPHQRRSIECCTERNECNKDLHPTLPPLKNRDFV

SEQ ID NO: 111 - protein, 352 aa, synthetic construct
KKEDGESTAPTPRPKVLRCYCSAGHCPEDSVNNICSTDGYCFTMIEEDDSGLPVVTSGCMKYEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 356 aa, synthetic construct
KKEDGESTAPTPRPKVLRCKCHHHCPEDSVNNICSTDGYCFTMIEEDDSGLPVVTSGCMKYEGSDFQCRDTPIPHQRRSIECCTERNECNKDLHPTLPPVVIGPFFDGSIRTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 107 aa, synthetic construct
KKEDGESTAPTPRPKVLRCKCHAHHCPEDSVNNICSTDGYCFTMIEEDDSGLPVVTSGCLGLEGSDFQCRDTPIPHQRRSIECCTERNECNKDLHPTLPPLKNRDFV